(19) United States
(12) Patent Application Publication
(10) Pub. No.: US 2010/0028344 A1
(43) Pub. Date: Feb. 4, 2010

(54) CONDITIONED CELL IMMUNIZATION

(75) Inventor: George SCHREINER, Los Altos, CA (US)

Correspondence Address: COOLEY GODWARD KRONISH LLP;ATTN: Patent Group, Suite 1100, 777 - 6th Street, NW, WASHINGTON, DC, 20001, US

(73) Assignee: Raven Biotechnologies, Inc.

(21) Appl. No.: 12/579,871

(22) Filed: Oct. 15, 2009

Related U.S. Application Data
(63) Continuation of application No. 11/773,935, filed on Jul. 5, 2007.

(51) Int. Cl. A61K 39/395; A61K 39/00; C12Q 1/68; C12Q 1/02; C12N 5/02; C40B 30/04; A61K 38/00; A61K 31/7088

(52) U.S. Cl. 424/133.1; 424/130.1; 424/178.1; 424/184.1; 435/6; 435/29; 435/375; 506/9; 514/2; 514/44 R; 514/44 A

(57) ABSTRACT

The present invention provides methods and compositions for generating modulators capable of binding to antigens presented by a cell that has been exposed to cellular conditioning. The present invention also includes methods and compositions for the prevention, treatment and diagnosis of disorders using the antigen modulators. The present invention further provides methods for identifying novel molecular targets for the treatment of different disorders.

### RELATED APPLICATIONS

This application claims priority to U.S. Provisional Application No. 60/818,761, filed Jul. 5, 2006, and 60/858,921, filed Nov. 15, 2006, both entitled “Conditioned Cell Immunization,” which are herein incorporated by reference in their entirety.

### FIELD OF THE INVENTION

The present invention provides methods and compositions for generating modulators capable of binding to antigens displayed on a cell that has been exposed to cellular conditioning. The present invention also includes methods and compositions for treatment and diagnosis of conditions using the antigen modulators. The present invention further provides methods for identifying novel molecular targets for the treatment of different conditions.

### BACKGROUND OF THE INVENTION

Despite numerous advances in medical research, treatments for a large number of human diseases including many forms of cancer, inflammation, multiple sclerosis and other genetic disorders, remain merely palliative and not curative. In the case of cancer, traditional modes of clinical care, such as surgical resection, radiotherapy, and chemotherapy have a significant failure rate, especially for solid tumors. Increasingly, incidences of treatment failure occur not only when a tumor is initially unresponsive, but also when even responsive tumor cells develop resistance to their chemical and radiation treatment regimens. In some cases, conventional therapies may eliminate the majority of cells within a tumor. However, a small number of cells often become resistant to the therapies and reestablish the aberrant pattern of proliferation. The resistant cells can and often do proliferate at a rate more rapid than that of the primary tumor cells, leading to recurrence of even more malignant tumors for which no existing chemo- and radio-therapy are effective. In some instances, tumor cells treated with chemotherapeutic drugs develop cross-resistance to other cytotoxic agents to which they have never been exposed, effectively eliminating the possibility of treating these tumors with chemotherapy as a whole. Similar observations of an increasing resistance to standard treatments has been observed with a wide range of diseases and disorders, such as the increasing antibiotic resistance observed for pneumonia and other pathogen infections.

The precise molecular mechanism of failure in the first-line treatment of many diseases remains largely elusive. However, it has been reported that disease cells, such as neoplastic cells, respond to exogenous stimuli and agents differently than normal cells. For example, tumor cells have been found to differentially express certain proteins in response to hypoxia (Yin et al. Cancer Res. (2006) 66: 2937-2945; Vihanto et al. FASEB Journal (express article 10.1096/fj.04-3647fe., published online Aug. 4, 2005)), as well as certain chemotherapeutic agents (Rubinfeld et al. Nature Biotechnology (2005) 24: 205-209; US Patent Application No. 20060003960; Chen et al. Cancer Letters (2002) 181: 95-107; Yang et al. Clin Cancer Res (2005) 11: 6226-6232; Cecconi et al. Journal of Proteone Research (2005) 4: 1909-1916).

Of particular interest are the proteins differentially expressed in response to a given medical treatment or external stimulus that are cell surface antigens. Cell surface antigens constitute a large family of proteins, glycoproteins, polysaccharides, and lipids. It is known that cell surface antigens serve not only as structural constituents of the plasma membrane, but more importantly they participate in transducing signals triggered by external stimuli such as growth factors, hormones, and various pathogens in the cells. Perturbations of cell surface antigen expression and the post-translational modification of surface antigens have long been acknowledged to account for a number of diseases including numerous forms of cancer, vascular diseases, neuronal, infectious, and endocrine diseases. Changes in cell surface antigen expression and other genetic and epigenetic changes also can occur in response to administered exogenous agents as well as pathogenic stimuli. For example, it has been reported that an antibody to a cell surface antigen can abrogate the protective effect that antigen appeared to normally confer on a cell against apoptosis the researchers had induced in that setting with irradiation or the chemotherapeutic agent doxorubicin (Y.-T. Tai et al., Experimental Hematology (2002) 30:212-220).

Researchers have reported that chemotherapeutic agents alter the expression of certain genes, upregulating some gene products and downregulating others. Rubinfeld et al., Nature Biotechnology (2006) 24(2):205-209, compared gene transcripts of tumor xenografts grown in mice treated with the chemotherapeutic agent CPT-11, looking for transcripts coding for putative cell-surface proteins that underwent significant upregulation relative to controls. U.S. Pat. App. 20060003960 discloses methods of treating tumors combining the administration of a chemotherapeutic agent and an antagonist of a gene product the expression of which is upregulated by the chemotherapeutic agent.

The present invention recognizes that differences in gene, protein, epitope and antigen expression exist between normal and diseased, damaged or injured cells following exposure to standard therapies and environmental factors. For example, a cell surface antigen preferentially induced in cancer cells following drug treatment might serve as a target for modulator, antagonist or agonist, such as, for example, a therapeutic antibody or a small molecule, used in combination with that drug. Such antigen modulators may also include, for example, radioisotope conjugates and toxin conjugates. In addition, having an understanding of the genetic processes engaged by treated or exposed cells can reveal new markers for efficacy and prognosis as well as further insights into the mechanisms of drug action that lead to the identification of new therapies.

There remains a considerable need for a new treatment paradigm focusing on the changes that diseased or damaged cells are likely to undergo upon exposure of a therapeutic agent or other external stimuli. The present invention addresses this need and provides related advantages as well. One way this need is addressed is through the use of antibodies.

In addition to their known uses in diagnostics, antibodies have been shown to be useful as therapeutic agents. For example, immunotherapy, or the use of antibodies for therapeutic purposes has been used in recent years to treat cancer. Passive immunotherapy involves the use of monoclonal antibodies in cancer treatments. See for example, Cancer: Principles and Practice of Oncology, 6^{th} Edition (2001) Chapt. 20 pp. 495-508. These antibodies can have inherent therapeutic biological activity both by direct inhibition of tumor cell growth or survival and by their ability to recruit the natural cell killing activity of the body's immune system. These agents can be administered alone or in conjunction with radiation or chemotherapeutic agents. Rituximab and Trastuzumab, approved for treatment of non-Hodgkin's lymphoma and breast cancer, respectively, are two examples of such therapeutics. Alternatively, antibodies can be used to make antibody conjugates where the antibody is linked to a toxic agent and directs that agent to the tumor by specifically binding to the tumor. Gemtuzumab ozogamicin is an example of an approved antibody conjugate used for the treatment of leukemia. Monoclonal antibodies that bind to cancer cells and have potential uses for diagnosis and therapy have been disclosed in publications. See, for example, the following patent applications which disclose, inter alia, some molecular weights of target proteins: U.S. Pat. No. 6,054,561 (200 kD c-erbB-2 (Her2), and other unknown antigens 40-200 KD in size) and U.S. Pat. No. 5,656,444 (50 kD and 55 kD oncofetal protein). Example of antibodies in clinical trials and/or approved for treatment of solid tumors include: Trastuzumab (antigen: 180 kD, HER2/neu), Edrecolomab (antigen: 40-50 kD, Ep-CAM), Anti-human milk fat globules (HMFG1) (antigen >200 kD, HMW Mucin), Cetuximab (antigens: 150 kD and 170 kD, EGF receptor), Alemtuzumab (antigen: 21-28 kD, CD52), and Rituximab (antigen: 35 kD, CD20).

The antigen targets of trastuzumab (Her-2 receptor), which is used to treat breast cancer, and cetuximab (EGF receptor), which is used for the treatment of several cancers, are present at some detectable level on a large number of normal human adult tissues including skin, colon, lung, ovary, liver, and pancreas. The margin of safety in using these therapeutics is possibly provided by the difference in the level of expression or in access of or activity of the antibody at these sites. In addition to cancer targets, antibody therapeutics have also been shown to be effective against chronic inflammation and other immune disorders. An example of an antibody therapeutic approved for treatment of immune disorders is infliximab (antigen: TNF-alpha).

Another type of immunotherapy is active immunotherapy, or vaccination, with an antigen present on a specific cancer(s) or a DNA construct that directs the expression of the antigen, which then evokes the immune response in the individual, i.e., to induce the individual to actively produce antibodies against their own cancer. Active immunization has not been used as often as passive immunotherapy or immunotoxins.

Several models of disease (including cancer) progression have been suggested. Theories range from causation by a single infective/transforming event to the evolution of an increasingly “disease-like” or ‘cancer-like’ tissue type leading ultimately to one with fully pathogenic or malignant capability. Some argue that with cancer, for example, a single mutational event is sufficient to cause malignancy, while others argue that subsequent alterations are also necessary. Some others have suggested that increasing mutational load and tumor grade are necessary for both initiation as well as progression of neoplasia via a continuum of mutation-selection events at the cellular level. Some cancer targets are found only in tumor tissues, while others are present in normal tissues and are up regulated and/or over-expressed in tumor tissues. In such situations, some researchers have suggested that the over-expression is linked to the acquisition of malignancy, while others suggest that the over-expression is merely a marker of a trend along a path to an increasing disease state.

An ideal diagnostic and/or therapeutic antibody would be specific for an antigen present on a large number of cancers or diseased tissues, but absent or present only at low levels on any normal tissue. The discovery, characterization, and isolation of a novel antigen that is specifically associated with cancer(s) would be useful in many ways. First, the antigen could be used to make monoclonal antibodies against the antigen. An antibody would ideally have biological activity against cancer cells and be able to recruit the immune system's response to foreign antigens. An antibody could be administered as a therapeutic alone or in combination with current treatments or used to prepare immunoconjugates linked to toxic agents. An antibody with the same specificity but with low or no biological activity when administered alone could also be useful in that an antibody could be used to prepare an immunoconjugate with a radio-isotope, a toxin, or a chemotherapeutic agent or liposome containing a chemotherapeutic agent, with the conjugated form being biologically active by virtue of the antibody directing the toxin to the antigen-containing cells.

What is needed are processes that identify accessible targets or antigens on the surface of diseased, damaged, or injured cells. What is also needed are novel, accessible targets on the surface of diseased, damaged, or injured cells that may be used to diagnose and treat, as well as monitor treatment efficacy and prognosis of, such diseases and/or damage with antibodies and other antigen modulators that specifically recognize the cell surface targets. As will be described in more detail below, the present inventors have made discoveries concerning the use of biological modifiers such as cell conditioning agents to identify novel antigens that are identified as the antigen targets of the novel antagonists, modulators and antibodies provided herein.

### SUMMARY OF THE INVENTION

The inventions as described herein are generally related to various methods of treating a disease, disorder or injury comprising administering cell conditioning agents and modulators of an antigen displayed on the surface of a cell which is altered in a diseased, damaged or injured cell relative to corresponding normal cells by the cell conditioning agents. This invention focuses on the interface between such conditioned cells and their extracellular milieu. The described inventions include various methods of identifying and using such antigens, methods of identifying and using such modulators and various other treatment, diagnostic and other methods, compositions and agents as described throughout this application.

### BRIEF DESCRIPTION OF THE DRAWINGS

FIG. 1 is a graphical representation of colon carcinoma cells conditioned with 200 rads of radiation and non-irradiated cells (control) treated with a dose response of an antibody generated against radiation conditioned rectal carcinoma cells. The graphed results show total ATP in the colon carcinoma cells.

FIG. 2 is a graphical representation of lung adenocarcinoma cells conditioned with 200 rads of radiation and non-irradiated cells (control) treated with a dose response of an antibody generated against radiation conditioned colon carcinoma cells.

FIG. 3 shows a representative graph of the RFU for breast carcinoma cells treated with Antibody 3 and with taxotere

### INCORPORATION BY REFERENCE

All publications, patents and patent applications mentioned in this specification are herein incorporated by reference to the same extent as if each individual publication, patent or patent application was specifically and individually indicated to be incorporated by reference.

### DETAILED DESCRIPTION OF THE INVENTION

The present invention is, at least in part, based on the recognition that qualitative and/or quantitative differences in antigens displayed on the surface of a cell exist between normal and diseased, damaged or injured cells following exposure to standard care therapeutics or other conditioning agents that can be exploited to provide new combination therapies. For example, a cell surface antigen preferentially induced in cancer cells following drug or radiation treatment might serve as a target for modulator of that cell surface antigen, such as, for example, a therapeutic antibody or a small molecule, used alone or in combination with that drug. In addition, having an understanding of the biological modifications undergone by treated cells can provide new markers for efficacy and prognosis as well as further our understanding the mechanisms of drug action.

It is an object of the present invention to identify and create monoclonal antibody drugs and other antigen modulators directed to targets whose expression and presence are altered by cell conditioning agents such as chemotherapies, targeted small molecules, biologics, and radiation. Input cell lines, including progenitor, stem cell and tumor-derived cell lines, have been shown to be useful immunogens to create cell surface directed antigen-specific antibodies. Treating these cells or cell lines with a biological modifying agent before starting the immunization, and screening for those antigens newly present, present in altered form, present in a different cellular location, or present to a different degree, on the biologically modified (conditioned), but not on the un-conditioned, cells, enables the rapid selection of one or more, or a set of, MAbs specific for antigens of interest useful for further screening, for internalization into a target cell, and for in vitro and in vivo activity.

This invention, by its focus on the membrane changes of a conditioned cell, is especially useful for whole cell immunization and the discovery of therapeutic antibodies and their targets; antibodies and other extracellular antigen modulators bind to membrane epitopes and cannot recognize intracellular epitopes. This is a significant advantage over other methods that ascertain intracellular changes induced by cellular conditioning.

In one aspect, the present invention provides cell surface antigens presented by a conditioned cell. This invention particularly focuses on the use of modulators of cell surface antigens the presence of which has been determined to be selectively altered in a diseased, damaged or injured cell relative to corresponding normal cells by a cell conditioning agent. Analysis of the cellular response to different treatments provides insight into a variety of diseases. These analyses can provide tests for early detection, diagnosis and prognosis of cells that may be relatively susceptible or resistant to the treatment. In addition, these analyses also provide new targets of therapeutic utility.

According to the teachings of this invention, treatments are targeted to the diseased, damaged or injured cell, and they are also targeted to normal cells located near to diseased, damaged or injured cells.

The present invention provides therapeutic and diagnostic methods, compositions and pharmaceutical packages for conditions involving injury, disease, disorders, or cellular damage or change. Specifically, the present invention provides methods comprising administering an antigen modulator to a subject who has been or may be subjected to cellular conditioning. The antigen modulator typically interacts with a cell surface antigen that is presented by a cell from a subject who has been exposed to said cellular conditioner, and is effective in assessing, preventing or ameliorating said disease, damage, or disorder in conjunction with the effects of the cellular conditioner or the conditioning therapeutic intervention. In certain embodiments, the antigen modulator is effective in ameliorating the disease condition prior to, subsequent to, or upon initiation of the cellular conditioning. In specific embodiments, the subject has been exposed to a therapeutic cellular conditioning involving an anti-cancer therapeutic regime including but not limited to one or more chemotherapeutic agents and/or radiation. In a related embodiment, the method involves administering to a subject diagnosed with a disease an amount of a chemotherapeutic agent prior to, subsequent to, or concurrent with the administration of an antigen modulator of a cell surface antigen that is differentially expressed in a diseased or damaged cell relative to corresponding normal cells that are not exposed to said chemotherapeutic agent.

Cells to be conditioned according to the methods of this invention may be optimized for differential antigen expression or for other desired features. In some embodiments, fetal cells (stem, progenitor or differentiated) are selected for conditioning when the desired antigens are onco-fetal antigens or other antigens presented de novo by cells at a specific stage of differentiation. Adult cells or tumor cells are also preferentially selected in certain embodiments. Cells from particular tissues of interest are selected in certain embodiments, as are cells from tumor, immortalized or normal tissues. Antigen presenting cells are also useful for the practice of the claimed invention.

In a separate embodiment, the present invention provides a method of inhibiting growth of a neoplastic cell, comprising administering to a subject in need thereof a pharmaceutical composition comprising an antigen modulator that binds to a cell surface antigen a cell from a subject, who has been exposed to an anti-cancer therapeutic agent, wherein said antigen modulator, alone or in combination with other agents, is effective to assess, prevent, or inhibit growth of, or kill the neoplastic cell. A wide variety of anti-cancer agents can be employed in the subject methods. Non-limiting examples are chemotherapeutic agents, cytostatic agents, cytotoxic agents, and non-peptide small molecules such as Gleevec (imatinib mesylate), Velcade (bortezomib), Casodex (bicalutamide), Iressa (gefitinib), gemcitabine, platinum agents such as oxaliplatin and cisplatin, taxanes such as taxol and taxotere, doxorubicin, Temodar (temozolomide) and Adriamycin. The present invention also provides a method of maintaining or increasing cell susceptibility to a cellular conditioner comprising delivering to a subject in need thereof a therapeutically effective amount of an antigen modulator specific for a cell surface antigen that is differentially expressed in response to said cellular conditioner, said administration being prior to, subsequent to, or in combination with said cellular conditioner, wherein said antigen modulator alone is effective to inhibit growth of target cells in said subject prior to, subsequent to, or upon exposure to said cellular conditioner.

In another embodiment, the antigen modulator specific for a cell surface antigen that is differentially expressed in response to said cellular conditioner acts on the target cells to cause increased susceptibility to host immune surveillance. In some instances host immune surveillance may involve complement dependent cytotoxicity (CDC) or antibody dependent cell mediated cytotoxicity (ADCC). In other instances the antigen modulator may cause an increased recognition of target cells by immune surveillance.

In another aspect, this invention provides methods for diagnosing the degree to which a subject has been exposed to a cell conditioning agent, comprising (a) obtaining a sample of cells from said subject; and (b) exposing said cells to at least one modulator of a cell surface antigen the presence of which is selectively altered in a target cell relative to normal cells by said cell conditioning agent. These methods are particularly suited to diagnosing and triaging civilians, soldiers and others who may have been exposed to radiation or chemical or biological weapons. In some aspects of these methods, the cell sample is exposed to a panel of at least one antigen modulator, each such modulator being specific for at least cell surface antigen the presence of which is selectively altered in a target cell relative to normal cells by varying exposures to said cell conditioning agent. In some instances, the cell sample is selected from the group consisting of bone marrow cells, peripheral white blood cells, red blood cells, tumor cells, and endothelial cells.

In yet another aspect, this invention provides methods for assessing and quantitating the degree to which an efficacious dose of radiation, chemotherapy or other cellular conditioning has been attained. Post-dosing samples of diseased or peripheral tissues are obtained and stained for the presence of treatment-induced antigens. In certain aspects, this method involves obtaining a sample of cells from a subject, and then determining the presence in that sample of at least one cell surface antigen the presence of which is selectively altered in a target cell relative to normal cells by said cell conditioning agent. Suitable cells for sampling include, without limitation, bone marrow cells, peripheral white blood cells, red blood cells, tumor cells, and endothelial cells. Detection of cell surface antigen presence is desirably accomplished using a modulator specific for said cell surface antigen obtained using the methods of this invention. These antigens are useful as biomarkers to assess the degree to which an efficacious dose of said cell conditioning agent has been attained.

In other aspects, this invention provides methods to enable the selection of responsive patients for treatment. In these embodiments, for example, an antibody or other modulator specific for at least one antigen the presence of which is selectively altered in a target cell relative to normal cells by a cell conditioning agent, is detectably labeled and administered to a patient who is also receiving a therapeutic cell conditioning agent. The detectable label is then imaged or otherwise detected, showing that the modulator is hitting the target. If desired, additional administration of the modulator and/or the cellular conditioning agent may then be administered. Suitable detectable labels are well known in the art, and include without limitation, radioisotopes, fluorescent agents, enzyme-linked agents, color-changing agents, and bioluminescent agents, and affinity-based agents. In some aspects of this invention, subsequent treatments given after the initial detection are with antigen modulators that are the same or different from the initially given labeled modulator. In some embodiments, the cell conditioning agent is administered consecutively with the modulator, and in other embodiments the administration is concurrent.

In some aspects of this invention, the antigen modulators are used to protect normal tissue near a diseased or damaged tissue. For example, using the methods of this invention, it is determined that certain diseased cells do not express an antigen, or express the antigen in an altered fashion, but that normal cells located in vivo near the diseased or damaged tissue do express the antigen. In such embodiments, an antigen modulator is administered to serve as an agonist modulator, providing trophic or protective stimulation of the normal cells while the diseased or damaged cells are treated by the cell conditioning agent. These embodiments are particularly useful in protecting endothelial tissue, bone marrow, blood, liver tissue etc. during radiation or chemotherapy of cancer. The methods of this invention permit the selective enhancement of the expression or a gene or surface antigen in a diseased cell, comprising the administration to that cell of a cellular conditioning agent. Gene profiling and antigen profiling of conditioned cells are useful aspects of this invention.

In certain embodiments of this invention, the antigen modulators are used to target the normal tissue near a diseased or damaged tissue. For example, using the methods of this invention, it is determined that, upon exposure to a cellular conditioner, normal cells located in vivo near the diseased or damaged tissue, such as endothelial cells, do express an antigen to an extent or in a form not expressed by the nearby diseased cells and not expressed by normal cells not located near diseased or damaged tissue. In such embodiments, an antigen modulator is administered to the normal, conditioned tissue. Administration of such a modulator serves to weaken or damage the cellular structures that support or maintain a diseased cell, thus injuring the diseased cell. For example, an antigen modulator of this invention is administered to conditioned endothelial cells near a tumor, resulting in damage to the tumor vasculature and the inhibition of angiogenesis.

The methods of this invention are suitable for tailoring the precise location for effective administration of a cell conditioning agent, and minimizing non-specific targeting of the cellular conditioner. This is especially useful when the modulator administered to the conditioned target cell is an immunoconjugate or carries a toxic moiety (such as a toxin or radioisotope) that is sensitive to binding or local release from the modulator upon administration of the cell conditioning agent or other instigator. For instance, the toxic moiety is released locally in the tissue targeted by the modulator while minimizing non-specific metabolism of the toxic moiety beyond the target diseased or damaged tissue.

Also included in the present invention is a method of providing a targeted treatment of a condition such as inflammation, infection or neoplasia that is susceptible to treatment with radiation or other cellular conditioners. The method involving radiation as the cellular conditioner typically involves the steps of a) radiating an area suspected of the condition; and b) delivering an antigen modulator that binds to a cell surface antigen differentially expressed in response to said radiation, thereby providing a targeted treatment. In certain embodiments, the step (a) is effected by a dose of radiation sufficient to induce expression of the cell surface antigen, which may be but is not necessarily a sub-therapeutic level. In other embodiments, the step (b) is effected by a dose of the antigen modulator effective to ameliorate said condition alone upon initiation of step (a). Where desired, step (b) further comprises applying radiation to the area of suspected condition.

The present invention also provides a method for the preparation of a cellular vaccine, in which damaged or diseased cells are exposed to levels of radiotherapy and/or chemotherapy, or exposed to a cellular conditioner such as but not limited to an infectious agent, hypoxia, heat or electric charge, and oxidation injury; wherein the exposure is sufficient to induce cell surface antigens that are qualitatively or quantitatively different relative to a corresponding unconditioned cell. In a related embodiment, the present invention provides a composition for vaccinating against disease such as cancer, infection or inflammation, comprising at least one target cell that is expressing a cell surface antigen the expression of which has been determined to be selectively altered in a damaged or diseased cell relative to corresponding normal cells by the exposure of said target cell to levels of radiotherapy and/or chemotherapy, or exposure to a cellular conditioner such as but not limited to an infectious agent, hypoxia, heat or electric charge, oxidation injury and the like. In related embodiments, the invention presents novel methods for presenting an immunizing epitope. In other embodiments, the invention provides purified cell membrane preparations derived from the conditioned cells of this invention that serve as a veccine to induce or augment a subject's endogenous immune response.

In a related embodiment, the methods of this invention involve additional steps to identify the alterations in gene expression or alterations in cell surface antigen profiles that result from cellular conditioning. These methods may involve the steps of: (a) incubating a diseased cell with a dose of a radiotherapeutic or other cell conditioning agent; (b) determining the gene expression profile or the surface antigen profile of said disease cell prior to and following said incubation; (c) identifying a gene or surface antigen the expression or presence of which is altered by said agent; and (d) treating said patient with a combination of a cell conditioning agent and an antigen modulator targeting said gene or cell surface antigen. Moreover, provided in the present invention is a therapeutic composition comprising an effective amount of a dose of a radiotherapeutic agent and an a modulator of a gene product encoded by a gene the expression of which is selectively upregulated in diseased cells relative to corresponding normal cells by said radiotherapeutic agent.

A wide variety of antigen modulators can be employed for any one of the subject treatment methods. Preferred antigen modulators include without limitation antibodies, antibody-like molecules, immunoconjugates, radioisotope conjugates, toxin conjugates, peptides, non-peptide small organic molecules, antisense molecules, inhibitory or interfering RNA and oligonucleotide decoys. The subject treatment and diagnostic methods provided herein are effective in treating a diversity of conditions, disorders, damage, injuries and diseases including but not limited to infectious disease, autoimmune disease, inflammation, cardiovascular disease, and neuronal disease.

Further provided is a method of immunizing a host mammal or an antibody-producing cell to produce an antibody that binds to a cell surface antigen presented by a conditioned cell, comprising: contacting the mammal or the antibody-producing cell with said conditioned cell under conditions suitable for eliciting an immune response. In a related embodiment, the present invention provides a method comprising: a) immunizing an antibody-producing cell with said conditioned cell to elicit an immune response culminating in production of said monoclonal antibody specific for said surface antigen; and b) processing said antibody-producing cell under conditions such that said monoclonal antibody is produced. Where desired, the step of processing involves generating a hybridoma cell that secretes the monoclonal antibody. A cell can be conditioned by exposing the cell in vivo or in vitro to a conditioning agent (such as but not limited to an anti-cancer agent, a chemotherapeutic agent or radiation), or to a cellular conditioner such as is provided by (without limitation) oxidation injury, hypoxia, temperature shift, electric charge, or infectious agent. The antibody-producing cell employed in the subject method may be a cell such as a lymphoid cell, such as a pre-B or B cell. In other embodiments, the conditioned cell, cellular membrane fragments and/or antigens derived therefrom may be used to screen for modulator molecules present in compound, CDR, peptide or other libraries of molecules, many of which are commonly or routinely available.

Additionally, the present invention provides a method for identifying a gene target for disease treatment comprising: (a) contacting a cell with a dose of a radiotherapeutic agent; (b) determining the gene expression profile of said cell; and (c) identifying a gene the expression of which is enhanced by said radiotherapeutic treatment relative to its expression in a corresponding untreated cell, as a target for disease treatment. The invention also provides a method for detecting genotypic changes in a cell, comprising profiling the cell surface antigen phenotype of a cell that has been exposed to radiotherapy, chemotherapy, and/or a cellular conditioner such as an infectious agent, hypoxia, heat shock, or an oxidation injury.

Diagnostic and therapeutic compositions and packaged pharmaceuticals for treating patients are provided, comprising a cell conditioning agent and a modulator specific for a cell surface antigen the presence of which has been determined to be selectively altered in a cell relative to untreated cells by said cell conditioning agent.

Further compositions and methods are provided herein.

### I. General Techniques and Definitions

The practice of the present invention will employ, unless otherwise indicated, conventional techniques of immunology, molecular biology, microbiology, cell biology and recombinant DNA, which are within the skill of the art. See, e.g., Sambrook, et al. MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. McPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As used in the present specification, the following words and phrases are generally intended to have the meanings as set forth below, except to the extent that the context in which they are used indicates otherwise.

As used in the specification and claims, the singular form “a”, “an” and “the” include plural references unless the context clearly dictates otherwise. For example, the term “a cell” includes a plurality of cells, including mixtures thereof.

The term “cell” as used herein refers to any structure containing nuclear and cytoplasmic material enclosed by a semipermeable membrane and, in plants, a cell wall. By way of example and without limitation, cells suitable for use in this invention include stem cells, progenitor cells, fetal or embryonic cells, adult cells, whether freshly isolated or cultured, and whether normal, tumor, damaged or diseased,

The term “conditioned cell” as used herein refers to a cell that has been exposed to a biological modifier or cellular conditioner resulting in at least one differentially expressed gene or protein, or in a qualitative or quantitative difference in at least one antigen displayed, expressed or presented on the cell surface as compared to an unexposed cell, such as at least one epitope or antigen the presence of which is altered in kind or in degree relative to a similar cell that has not been so exposed.

A “target antigen” as used herein refers to one or more antigens or epitopes the presence of which on a cell has been determined to be altered in a diseased, damaged or injured cell relative to corresponding normal cells by a cell conditioning agent. As used herein, “target antigen” also refers to a differentially expressed gene or protein, or the product of a differentially expressed gene, regardless of whether the alteration is due to expression or other modification. In some embodiments, the antigen may be non-proteinaceous in nature. For instance, in some embodiments of the methods of treatment, the modulator of the antigen may recognize or specifically bind a non-proteinaceous antigen, such as a carbohydrate residue or lipid.

The terms “differentially expressed” or “differential expression” as applied herein to a gene, protein, antigen or epitope, and their synonyms, which are used interchangeably, as applied to a nucleotide or polypeptide sequence, or a protein or epitope in a cell or subject, refer to a gene, protein, antigen or epitope whose expression or presence on a cell is induced, altered, enhanced, modulated, impaired, or activated to a higher or lower level as compared to a normal or control cell or subject. Differential expression also encompasses expression of a particular nucleotide sequence or protein even or any alteration described herein when there is no detectable base level of expression in a control cell or subject as well as elevated expression or upregulated expression as compared to a control cell, regardless of the control cell level of expression. Such control can be an untreated cell or a corresponding normal cell or subject, in comparison to a cell or a subject suffering from an injury, disease or disorder, depending on the context it is applied. The terms also include genes, proteins, antigens or epitopes whose expression is at a higher or lower level at different stages of the same disease or injury progression. The terms also include genes, proteins, antigens or epitopes whose expression is higher or lower in patients who are significantly sensitive or resistant to certain therapeutic regimen. The terms also include genes, proteins, antigens or epitopes that vary in sequence or post-translational modification from a normal or control nucleotides or proteins. It is also understood that a differentially expressed gene, protein, antigen or epitope may be either activated or inhibited at the nucleic acid level or protein level, or may be subject to alternative splicing or post-translational modification, to result in a different polypeptide product. Such differences may be evidenced by a change in mRNA levels, surface expression, secretion or other partitioning of a polypeptide, for example. For purposes of this invention, encompassed within the definition of a differentially expressed gene, protein, antigen or epitope are novel or enhanced proteins that occur as a result of biochemical modification, extracellular protease exposure, novel hetero-dimer formation, altered glycosylation, altered lipidation, clustering or patch formation of proteins, or changes in protein conformation, for example and without limitation, none of which are necessarily dependent on mRNA but all of which can create biochemical changes in a cell or subject that could produce novel or differentially expressed proteins, antigens or epitopes.

Differential gene, protein, antigen or epitope expression may include a comparison of expression between two or more genes, their gene products, or proteins, antigens or epitopes, or a comparison of the ratios of the expression between two or more genes, their gene products, antigens, epitopes or proteins, or even a comparison of two differently processed products of the same gene, which differ between normal subjects and subjects suffering from a disease, injury or disorder, or between various stages of the same disease. Differential expression includes both quantitative, as well as qualitative, differences in the temporal or cellular expression pattern in a gene, its expression products or proteins, antigens or epitopes, among, for example, normal and diseased cells, or among cells that have undergone damage or injury, disease events or different disease stages, or cells that are significantly sensitive or resistant to certain therapeutic regimen. For the purpose of this invention, “differential gene, protein, antigen or epitope expression” is considered to be present when there is at least an about a 10% preferably at least about 50%, more preferably at least about a two-fold, preferably at least about four-fold, more preferably at least about six-fold, most preferably at least about ten-fold difference between the expression of a given gene, protein, antigen or epitope in normal and diseased, damaged or injured subjects, or in various stages of damage development in a diseased, injured or damaged subject, or in patients who are differentially sensitive to certain therapeutic regimen.

The terms “altered,” “selectively altered,” “selectively upregulated”, “selectively enhanced” or “enhanced” or “activated” is used herein to refer to a gene, protein or antigen that is altered, upregulated, down-regulated, enhanced, activated or induced by at least 10% in a diseased, damaged or injured cell or subject by a given treatment or conditioning agent exposure as compared to a corresponding normal cell or tissue, such as in the same treated subject. Also encompassed within these terms are genes, proteins or antigens that are activated but not necessarily physically altered, such that their biology can be affected by an antibody or other modulator.

A “cell(ular) conditioner” or “cell(ular) conditioning” as used herein refers to a substance or external force in a biological setting, the addition or removal of which alters, impedes, stimulates, suppresses, enhances, impairs, interferes with, modifies, inhibits, upregulates or prevents the normal function of cells and/or causes injury, damage or destruction of cells. The term is intended without limitation to include radioactive isotopes (e.g. At⁻²¹¹, I⁻¹³¹, I⁻¹²⁵, Y⁻⁹⁰, Re⁻¹⁸⁶, Re⁻¹⁸⁸, Sm⁻¹⁵³, Bi⁻²¹², P⁻³² and radioactive isotopes of Lu), chemotherapeutic agents, toxins such as small molecule toxins or enzymatically active toxins of bacterial, fungal, plant or animal origin, including fragments and/or variants thereof. Cellular conditioning is also caused for example and without limitation, by increasing or decreasing the exposure of a cell to free radicals, electric charge, electric charge, ischemia, oxidant injury, heat shock, cardiac hypertrophy, fever, inflammation, metabolic diseases, infection, cytokines, growth factors, hormones, factor(s) released by tumor cells or damaged cells that re capable of eliciting new of up-regulated antigens, pathogens, (e.g., bacteria, parasites, fungi, viruses, prions, intracellular parasites such as malaria, and viroids), cell-cell interactions, soluble factors, and cell and tissue damage of other causes. In some embodiments, the terms “cellular conditioner” or “cellular conditioning” may be modified to exclude any one or more of the substances or external forces described above or herein. For instance, the term may include all of the substances or external forces with the exception of radiation and/or chemotherapeutic agents.

This invention further encompasses or uses cellular conditioners that are may be also considered ‘immune conditioners”, including but not limited to drugs such as cyclophosphamide, methotrexate, steroids (e.g. prednisone), anti-TNF agents and the like, that affect the membrane antigens of activated immune cells. These effects are detectable by the cell immunization methods of this invention.

As used herein, “agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

The term “antagonist” as used herein refers to a molecule having the ability to inhibit or reduce a biological function of a target polypeptide. Accordingly, the term “antagonist” is defined in the context of the biological role of the target polypeptide. While preferred antagonists herein specifically interact with (e.g. bind to) the target, molecules that inhibit a biological activity of the target polypeptide by interacting with other members of the signal transduction pathway of which the target polypeptide is a member are also specifically included within this definition. A preferred biological activity inhibited by an antagonist is associated with the development, growth, or spread of a tumor. Antagonists, as defined herein, without limitation, include antibodies and immunoglobulin variants, peptides, peptidomimetics, non-peptide small molecules, antisense molecules, interfering RNA molecules and oligonucleotide decoys.

The term “agonist” as used herein refers to a molecule having the ability to initiate or enhance a biological function of a target polypeptide. Accordingly, the term “agonist” is defined in the context of the biological role of the target polypeptide. While preferred agonists herein specifically interact with (e.g. bind to) the target, molecules that enhance a biological activity of the target polypeptide by interacting with other members of the signal transduction pathway of which the target polypeptide is a member are also specifically included within this definition. A preferred biological activity enhanced by an agonist is associated with the prevention or inhibition of the development, growth, or spread of a tumor or other diseased or damaged cell or tissue. For example, agonist ligand binding can stimulate the expression of a biological response modifier such as a protease that inhibits the growth or accumulation of a growth factor useful for the development of a tumor, such as by way of example and not by limitation, vascular endothelial growth factor. Agonists, as defined herein, without limitation, include antibodies and immunoglobulin variants, peptides, peptidomimetics, non-peptide small molecules, antisense molecules, and oligonucleotide decoys.

Agonists, antagonists, and other modulators of a differentially expressed antigen function are expressly included within the scope of this invention. In certain embodiments, the agonists, antagonists, and other modulators of a differentially expressed antigen are antibodies, antibody-like molecules and immunoglobulin variants that bind to the differentially expressed antigen. In other embodiments, these agonists, antagonists and modulators are polypeptides that comprise one or more of the antigenic determinant sites in a differentially expressed antigen, or comprise one or more fragments of such sites, variants of such sites, or peptidomimetics of such sites, as long as they retain the ability to bind to a native ligand of the differentially expressed antigen. These agonistic, antagonistic, and differentially expressed antigen modulatory compounds are provided in linear or cyclized form, and optionally comprise at least one amino acid residue that is not commonly found in nature or at least one amide isostere. These compounds may be glycosylated.

More specifically, the term “antigen modulator” or “modulator” as used herein refers to any substance natural or non-natural that can modulate the nature, activity and/or expression (including expression pattern and subcellular distribution) of a target antigen. The substance may be (1) capable of disrupting or blocking the interaction between a differentially expressed antigen and its native ligands or an antibody binding thereto; (2) capable of binding to the target antigen and its native ligands or an anti-antigen antibody; (3) contains an antigenic site that can be used in the raising of antibodies capable of binding to the target antigen and its native ligands or an anti-antigen antibody; (4) contains an antigenic site that can be used in the screening of antibodies capable of binding to the target antigen and its native ligands or an anti-antigen antibody; (5) contains an antigenic site that an be used in the raising of antibodies capable of disrupting or blocking the interaction between the target antigen and its native ligands or an anti-antigen antibody; (6) capable of binding to a target antigen and activating it such that prevention or inhibition of tumor growth occurs; (7) capable of activating complement on cytotoxic cells following antigen exposure; and/or (6) contains an antigenic site that can be used in the screening of antibodies capable of disrupting or blocking the interaction between the target antigen and its native ligands or an anti-antigen antibody. Antigen modulators may be “antigen agonists” or “antigen antagonists” depending on whether their activity enhances or inhibits normal antigen biological activity, respectively.

As used herein, the term “antigen variant” denotes any amino acid variant of a target antigen, including amino acid substitution, deletion, and addition variants, or any combination thereof. The definition encompasses chimeric molecules and other hybrid molecules. Also included in the definition is any fragment of a target antigen variant molecule that comprises the variant or hybrid region(s) of the molecule.

A binding molecule or member generally refers to one member of a pair of molecules that bind one another, one member being an antigen. The members of a binding pair may be naturally derived or wholly or partially synthetically produced. One member of the pair of molecules has an area on its surface, or a cavity, which binds to and is therefore complementary to a particular spatial and polar organization of the other member of the pair of molecules. Examples of types of binding pairs are antigen-antibody, biotin-avidin, hormone-hormone receptor, receptor-ligand, enzyme-substrate. In some embodiments, the present invention is generally concerned with antigen-antibody type reactions.

The term “effective amount” or “therapeutically effective amount” refers to that amount of as antagonist that is sufficient to effect beneficial or desired results, including with out limitation, clinical results as shrinking the size of the tumor (in the cancer context, for example, breast or prostate cancer), retardation of cancerous cell growth, delaying the development of metastasis, decreasing symptoms resulting from the disease, increasing the quality of life of those suffering from the disease, decreasing the dose of other medications required to treat the disease, enhancing the effect of another medication such as via targeting and/or internalization, delaying the progression of the disease, and/or prolonging survival of individuals. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular antagonist chosen, the dosing regimen to be followed, whether is administered in combination with other compounds, timing of administration, the tissue to be imaged, and the physical delivery system in which it is carried.

As used herein, “treatment” or “treating,” or “palliating” or “ameliorating” are used interchangeably herein. These terms refer to an approach for obtaining beneficial or desired results including and preferably clinical results. For purposes of this invention, beneficial or desired clinical results include, but are not limited to, one or more of the following: reducing the proliferation of (or destroying) cancerous cells or other diseased, reducing metastasis of cancerous cells found in cancers, shrinking the size of the tumor, decreasing symptoms resulting from the disease, increasing the quality of life of those suffering from the disease, palliating the pain resulting from the disease, decreasing the dose of other medications required to treat the disease, delaying the progression of the disease, and/or prolonging survival of individuals. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

“Surgery” means any therapeutic or diagnostic procedure that involves methodical action of the hand or of the hand with an instrument, or of a mechanical substitute, on the body of a human or other mammal, to produce a curative, remedial, or diagnostic effect.

“Radiation therapy” means exposing a patient, using routine methods and compositions known to the practitioner, to radiation emitters such as alpha-particle emitting radionuclides (e.g., actinium and thorium radionuclides), low linear energy transfer (LET) radiation emitters (i.e. beta emitters), conversion electron emitters (e.g. strontium-89 and samarium-153-EDTMP, or high-energy radiation, including without limitation x-rays, gamma rays, and neutrons.

An “anti-cancer agent” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository.

The term “monoclonal antibody” as used herein refers to an antibody which is directed against a single antigenic site or epitope and present in a population that is identical, except for possible mutations or substitutions, etc. that may be present in minor amounts. Furthermore, in contrast to conventional (polyclonal) antibody preparations that typically include different antibodies directed against different determinants (epitopes), each monoclonal antibody is directed against a single determinant on the antigen (antigenic site). The term “monoclonal antibody” encompasses not only intact monoclonal antibodies and full-length monoclonal antibodies, but also fragments thereof (such as Fab, Fab′, F(ab′)₂, Fv), single chain (ScFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen.

In addition to their specificity, monoclonal antibodies are advantageous in that they may be synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method (e.g., by hybridoma, phage selection, recombinant expression, transgenic animals, etc.). For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

“Cell surface antigens” or “antigens that are displayed on the cell surface” refers to the plasma membrane components of a cell, such as integral and peripheral membrane proteins, glycoproteins, polysaccharides, hydrophilic phosphorous molecules and lipids, and epitopes of the foregoing that are part of or become part of the plasma membrane. An integral membrane protein is a transmembrane protein that extends across the lipid bilayer of the plasma membrane of a cell. A typical integral membrane protein consists of at least one membrane-spanning segment that generally comprises hydrophobic amino acid residues. Peripheral membrane proteins do not extend into the hydrophobic interior of the lipid bilayer and they are bound to the membrane surface by noncovalent interaction with other membrane proteins. The cell surface antigens of this invention also constitute cytoplasmic proteins that are inserted into the cell membrase as a response to a conditioning agent. For example, certain heat-shock proteins translocate from their normal cytoplasmic location to the cell membrane via chaperone molecules. This transport may be induced by a cellular conditioning agent.

An “antigen” as used herein means a substance that is recognized and bound specifically by an antibody, a fragment thereof or by a T cell antigen receptor. Antigens can include peptides, proteins, glycoproteins, polysaccharides, carbohydrates and lipids; portions thereof and combinations thereof. The antigens can be those found in nature or can be synthetic. They may be present on the surface or located within a cell. The term “antigen” encompasses one or more epitopes of any of the foregoing entities, wherein the epitopes are linear or conformational in nature. In particular embodiments of the invention, the altered antigens (or epitopes) displayed of the surface of a cell that has been exposed to a conditioning agent are substantially novel in nature, not having previously been identified or not previously characterized in relation to a disease, disorder or injury. In some embodiments, the alteration to the antigen is qualitative in nature rather than quantitative in nature. As a non-limiting example, the glycosylation pattern of an antigen may be altered by a conditioning agent. In some embodiments, at least about 10%, 20%, 30%, 40%, 50%, 60%, 70%, 80%, 90%, 95% or more of the altered antigens (or epitopes) belong to such a class. As used herein, “target” antigens are synonymous with altered antigens.

An antigen or epitope that “specifically binds” or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an epitope that is target on a conditioned cell is an antibody that binds this conditioned cell epitope with greater affinity, avidity, more readily, and/or with greater duration than it binds to other conditioned cell epitopes or non-conditioned cell epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

The term “immunologically active” in reference to an epitope being or “remaining immunologically active” refers to the ability of an antibody to bind to the epitope under different conditions, for example, after the epitope has been subjected to reducing and denaturing conditions.

The term “antibody” as used herein refers to an immunoglobulin molecule and a fragment thereof, preferably an immunologically active portion of the immunoglobulin molecule, i.e., the fragment that contains an antigen-binding site that specifically binds (“immunoreacts with” or “is specific for”) an antigen. Structurally, the simplest naturally occurring antibody (e.g., IgG) comprises four polypeptide chains, two heavy (H) chains and two light (L) chains inter-connected by disulfide bonds. The immunoglobulins represent a large family of molecules that include several types of molecules, such as IgD, IgG, IgA, IgM and IgE. The term “immunoglobulin molecule” includes, for example, hybrid antibodies, or altered antibodies, and fragments thereof. It has been shown that the antigen binding function of an antibody can be performed by fragments of a naturally occurring antibody. These fragments can be broadly divided into “single-chain” (“Sc”) and “non-single-chain” (“Nsc”) types based on their molecular structures. Antibody-like molecules are similar in nature as disclosed above, typically comprising at least one antigen binding region or CDR of an antibody with the frame work or skeleton of another molecule such as albumin, serum proteins, etc.

Also encompassed within the terms “antibodies” are immunoglobulin molecules of a variety of species origins including invertebrates and vertebrates. The term “human” as applies to an antibody refers to an immunoglobulin molecule expressed by a human gene or fragment thereof. The term “humanized” as applies to non-human (e.g. rodent or primate) antibodies refer to hybrid immunoglobulins, immunoglobulin chains or fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences. These modifications are made to further refine and optimize antibody performance and minimize immunogenicity when introduced into a human body. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

“Non-single-chain” antibodies (“Nsc Abs”) are heteromultimers comprising a light-chain polypeptide and a heavy-chain polypeptide. Examples of the Nsc Abs include but are not limited to (i) a ccFv fragment stabilized by the heterodimerization sequences disclosed herein (described in U.S. Pat. No. 6,833,441); (ii) any other monovalent and multivalent molecules comprising at least one ccFv fragment as described herein; (iii) a Fab fragment consisting of the VL, VH, CL and CH1 domains; (iv) an Fd fragment consisting of the VH and CH1 domains; (v) an Fv fragment consisting of the VL and VH domains of a single arm of an antibody; (vi) an F(ab′)2 fragment, a bivalent fragment comprising two Fab fragments linked by a disulfide bridge at the hinge region; (vii) a diabody; and (viii) any other Nsc Abs that are described in Little et al. (2000) Immunology Today.

As noted above, a Nsc Ab can be either “monovalent” or “multivalent.” Whereas the former has one binding site per antibody, the latter contains multiple binding sites capable of binding to more than one antigen of the same or different kind. Depending on the number of binding sites, a Nsc Ab may be bivalent (having two antigen-binding sites), trivalent (having three antigen-binding sites), tetravalent (having four antigen-binding sites), and so on.

Multivalent Nsc Abs can be further classified on the basis of their binding specificities. A “monospecific” Nsc Ab is a molecule capable of binding to one or more antigens of the same kind. A “multispecific” Nsc Ab is a molecule having binding specificities for at least two different antigens. While such molecules normally will only bind two distinct antigens (i.e. bispecific Abs), antibodies with additional specificities such as trispecific antibodies are encompassed by this expression when used herein. Examples of bispecific antibodies include those with one arm directed against a tumor cell antigen and the other arm directed against a cytotoxic trigger molecule such as anti-FcgRI/anti-CD15, anti-p185^{HER}2/FcgRIII (CD16), anti-CD3/anti-malignant B-cell (1D10), anti-CD3/anti-p185^{HER2}, anti-CD3/anti-p97, anti-CD3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD19, anti-CD3/MoV18, anti-neural cell adhesion molecule (NCAM)/anti-CD3, anti-folate binding protein (FBP)/anti-CD3, anti-pan carcinoma associated antigen (AMOC-31)/anti-CD3; bispecific Abs with one arm which binds specifically to a tumor antigen and one arm which binds to a toxin such as anti-saporin/anti-Id-1, anti-CD22/anti-saporin, anti-CD7/anti-saporin, anti-CD38/anti-saporin, anti-CEA/anti-ricin A chain, anti-interferon-a (IFN-a)/anti-hybridoma idiotype, anti-CEA/anti-vinca alkaloid; BsAbs for converting enzyme activated prodrugs such as anti-CD30/anti-alkaline phosphatase (which catalyzes conversion of mitomycin phosphate prodrug to mitomycin alcohol); bispecific Abs which can be used as fibrinolytic agents such as anti-fibrin/anti-tissue plasminogen activator (tPA), anti-fibrin/anti-urokinase-type plasminogen activator (uPA); bispecific antibodies for targeting immune complexes to cell surface receptors such as anti-low density lipoprotein (LDL)/anti-Fc receptor (e.g. Fcg RI, FcgRII or FcgRIII); bispecific antibodies for use in therapy of infectious diseases such as anti-CD3/anti-herpes simplex virus (HSV), anti-T-cell receptor:CD3 complex/anti-influenza, anti-FcgR/anti-HIV; bispecific antibodies for tumor detection in vitro or in vivo such as anti-CEA/anti-EOTUBE, anti-CEA/anti-DPTA, anti-p185^{HER2}/anti-hapten; BsAbs as vaccine adjuvants (see Fanger et al., supra); and bispecific antibodies as diagnostic tools such as anti-rabbit IgG/anti-ferritin, anti-horse radish peroxidase (HRP)/anti-hormone, anti-somatostatin/anti-substance P, anti-HRP/anti-FITC, anti-CEA/anti-.beta.-galactosidase. Examples of trispecific antibodies include anti-CD3/anti-CD4/anti-CD37, anti-CD3/anti-CD5/anti-CD37 and anti-CD3/anti-CD8/anti-CD37.

“Single-chain antibodies” (“Sc Abs”) refers to a monomeric antibody. Although the two domains of the Fv fragment are coded for by separate genes, a synthetic linker can be made that enables them to be made as a single protein chain (i.e. single chain Fv (“scFv”) as described in Bird et al. (1988) Science 242:423-426 and Huston et al. (1988) PNAS 85:5879-5883) by recombinant methods. Other Sc Abs include antigen-binding molecules stabilized by heterodimerization sequences, and dAb fragments (Ward et al., (1989) Nature 341:544-546 ) which consist of a VH domain and an isolated complimentarity-determining region (CDR). Various linker sequences can also be used, and can provide additional functions, such as a means for attaching a drug or a solid support. A preferred single-chain antibody contains VL and VH regions that are linked together and stabilized by a pair of subject heterodimerization sequences. The scFvs can be assembled in any order.

An antibody “specifically binds to” or is “immunoreactive with” or “specific for” an antigen or epitope if it binds with greater affinity or avidity than it binds to other reference antigens or epitopes including polypeptides or other substances.

A “subject” as used herein refers to a biological entity containing expressed genetic materials. The biological entity is preferably a vertebrate, preferably a mammal, more preferably a human. The term “animal subject” as used herein includes humans as well as other mammals.

### II. Cellular Conditioning

In certain aspects, the present invention relates to the use of conditioned cells as immunogens, preferable intact and viable conditioned cells. Cells are conditioned either in vitro or in vivo, then harvested and used as an immunogen.

In certain embodiments, conditioned cells may be obtained by removing them from an individual (by surgical or other means) who has been exposed to a conditioning agent. In other embodiments, cells that have been removed from a subject may be conditioned ex vivo.

Suitable conditioned cells can be grown as a monolayer anchored onto a solid phase substrate, or as aggregates in a suspension culture. The choice of substrate is determined largely by the type of cell and the desired growth parameters (e.g. growth rate, desired density, media requirements etc.) Most cells can be propagated on a substrate made of e.g., glass, plastic or ceramic material. For certain cell types, such as neurons, epithelial and muscle cells, substrates pre-coated with charged substances that enhance cell attachment and spreading are preferred. Commonly employed coating materials include biological substrates that bear a net positive charge. Non-functioning examples of biological substrates include extracellular matrix/adhesion proteins such as lamirn, fibronectin, collagen, or synthetic polypeptide such as poly-lysine. A variety of non-biological substrates such as membranes made of nitrocellulose, nylon, polytetrafluoroethylene, or any other implant materials can also be used to support growth of cells in a serum-free medium.

Precautions are generally taken to maintain membrane integrity and preserve cell membrane components when harvesting cells cultured on different substrates. Unlike the traditional method of dissociating the anchored cells or cell layers by the action of strong proteolytic enzymes such as serine proteinase, trypsin, cell immunogens of the present invention are typically removed from the culture substrates by agents that minimize damages to the cell surface antigens. These agents include chelating agents, such as EDTA and EGTA, which bind to divalent metal ions (e.g. calcium and magnesium) known to be necessary for cell-substrate attachment. Other suitable cell dissociation agents encompass collagenases, dispases, and neutral proteinases when used in conjunction with serine proteinase inhibitors (e.g. soybean trypsin inhibitor). Treatment of cells with these agents mostly results in disruption of the extracellular matrix components while preserving the cell surface proteins. The time required to detach the cells anchored on a solid substrate can vary depending on the protease enzymes chosen, but will normally be a period of about 1 minutes to 30 minutes, and preferably about 5 minutes to 15 minutes. The enzymatic treatment can be carried out at room temperature or at about 37° C. Excess enzyme can be removed by gentle washing with buffers having pH and salt concentrations in the physiological range that are routinely prepared by one skilled in the art.

Prior to immunization, cell viability may be confirmed by the measurement of membrane integrity although cell membrane fragments maybe used for immunization. The methods for assessing membrane integrity are known in the art. The most common assay involves staining cells with a dye that reacts with either living or dead cells. As is apparent to one skilled in the art, exemplary dyes include trypan blue, propidium iodide, eosin Y, naphthalene black, nigrosin, erythrosin B and fast green.

Cells for immunization may be lightly fixed before immunization according to known methods, to the extent that the fixation does not alter the native conformation of the conditioned antigen or result in the loss of the differentiation of the expresses antigen compared to cell that has not been fixed.

Cells of any original, preferably those cells that are capable of growth in tissue culture, are candidate cells for conditioning. Non-limiting examples of specific cell types that can be grown in culture include connective tissue elements such as fibroblast, skeletal tissue (bone and cartilage), skeletal, cardiac and smooth muscle, epithelial tissues (e.g. liver, lung, breast, skin, bladder and kidney), neural cells (glia and neurons), endocrine cells (adrenal, pituitary, pancreatic islet cells), melanocytes, and many different types of hematopoetic cells. Cells in culture can be freshly isolated from body tissues (known as primary culture) or subcultured by expansion and/or cloning of the cells present in the primary culture (known as cell lines).

Suitable cells for conditioning can be any animal and plant cells. Preferred exemplary animal cells are cells are isolated from human, mouse, rat, fruit fly, worm, and exemplary plant cells include yeast, corn and rice. Cells applicable for conditioning can also be cells representative of a specific body tissue from a subject. The types of body tissues include but are not limited to blood, muscle, nerve, brain, heart, lung, liver, pancreas, spleen, thymus, esophagus, stomach, intestine, kidney, testis, ovary, hair, skin, bone, breast, uterus, bladder, spinal cord and various kinds of body fluids. Cells of different developmental stages (embryonic or adult) of an organism, or more specifically of various developmental origins including ectoderm, endoderm and mesoderm, can also be applied. Another type of cells embodied in the present invention is a “personal cell type”, which comprises cells derived from individuals of a family, or individuals from different generations within the same pedigree.

Of particular interest are cells that are associated with a particular disease or with a specific disease stage, treated cells derived from natural and induced immune deficiency states, cardiovascular disease, neuronal disease, inflammation states and diseases caused by a variety of pathogens. The association with a particular disease or disease stage may be established by the cell's aberrant behavior in one or more biological processes such as cell cycle regulation, cell differentiation, apoptosis, chemotaxis, cell motility and cytoskeletal rearrangement. A disease cell may also be confirmed by the presence of a pathogen causing the disease of concern (e.g. HIV for AIDS and HBV for hepatitis B).

Additional example of conditioned cells suitable as immunogens include treated neoplastic cells. In certain embodiments, the cancerous cells are selected from the group including but not limited to adrenal gland tumors, AIDS-associated cancers, alveolar soft part sarcoma, astrocytic tumors, bladder cancer (squamous cell carcinoma and transitional cell carcinoma), bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, dhordoma, chromophobe renal cell carcinoma, clear cell carcinoma, colon cancer, colorectal cancer, cutaneous benign fibrous histiocytomas, desmoplastic small round cell tumors, ependymomas, Ewing's tumors, extraskeletal myxoid chondrosarcoma, fibrogenesis imperfecta ossium, fibrous dysplasia of the bone, gallbladder and bile duct cancers, gestational trophoblastic disease, germ cell tumors, head and neck cancers, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), medulloblastoma, melanoma, meningiomas, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, synovial sarcoma, testicular cancer, thymic carcinoma, thymoma, thyroid metastatic cancer, and uterine cancers (carcinoma of the cervix, endometrial carcinoma, and leiomyoma). In certain preferred embodiments, the cancerous cells are selected from the group of solid tumors including but not limited to breast cancer, colon cancer, prostate cancer, lung cancer, sarcoma, renal metastatic cancer, thyroid metastatic cancer, and clear cell carcinoma.

Other categories of the subject cell immunogens are “genetically altered” or “chemically treated” cells. A cell is “genetically altered” as compared to a wildtype cell when a genetic element has been exogenously introduced into the cell other than by mitosis or meiosis. The element may be heterologous to the cell, or it may be an additional copy or improved version of an element already present in the cell. Genetic alteration may be effected, for example, by transfecting a cell with a recombinant plasmid, or other polynucleotide delivery vehicle through any process known in the art, such as electroporation, viral infection, calcium phosphate precipitation, or contacting with a polynucleotide-liposome complex. When referring to genetically altered cells, the term refers both to the originally altered cell, and to the progeny thereof. A preferred altered cell is one that carries a reporter gene to effect drug screening, cellular pathway delineation, and/or antibody selection.

A wide array of therapeutic agents can be employed to condition a cell either in vivo by directly administering the agents to a subject or in vitro by applying the agents to a cell in culture.

To condition a cell according to the practice of this invention, a cell is exposed to a cellular conditioner (which can be done at varying concentrations or dosages and/or different times), which as used herein refers to a substance or external force in a biological setting, the addition or removal of which alters, impedes, stimulates, suppresses, enhances, impairs, interferes with, inhibits or prevents the normal function of cells and/or causes injury, damage or destruction of cells. The term is intended without limitation to include radioactive isotopes (e.g. At⁻²¹¹, I⁻¹³¹, I⁻¹²⁵, Y⁻⁹⁰, Re⁻¹⁸⁶, Re⁻¹⁸⁸, Sm⁻¹⁵³, Bi⁻²¹², P⁻³² and radioactive isotopes of Lu), chemotherapeutic agents, toxins such as small molecule toxins or enzymatically active toxins of bacterial, fungal, plant or animal origin, including fragments and/or variants thereof. Cellular conditioning is also caused for example and without limitation, by increasing or decreasing the exposure of a cell to free radicals, electric charge, ischemia, oxidant injury, heat shock, cardiac hypertrophy, fever, inflammation, metabolic diseases, infection, cytokines, growth factors, hormones, pathogens, (e.g., bacteria, parasites, fungi, viruses, prions, intracellular parasites, and viroids), cell-cell interactions, soluble factors, and cell and tissue damage of other causes. The specific dose and duration of such conditioning will depend on the choice of the conditioning agents. One skilled in the art can readily observe an enhanced or altered expression of antigens in response to such conditioning by performing a variety of routine techniques for comparative protein profiling. Exemplary techniques include but are not limited to a number of immunoassay including Western blotting, ELISA and immunoprecipitation, mass spectrometry, and SDS-PAGE analysis. Where desired, nucleic acid analyses applicable for detecting differential expression of mRNA can be employed. Such analyses may involve hybridization of a vast number of gene probes immobilized on a DNA array, quantitative PCR, and SAGE analysis, all of which are known in the art.

Arrays of conditioned cells may be made, the array defined by different conditioning agent exposure variables. Such variables include, but are not limited to agent concentration, dosage and/or time or duration of exposure.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g. At⁻²¹¹, I⁻¹³¹, I⁻¹²⁵, Y⁻⁹⁰, Re⁻¹⁸⁶, Re⁻¹⁸⁸, Sm⁻¹⁵³, Bi⁻²¹², P⁻³², and radioactive isotopes of Lu), Suitable radiation sources for use as a cell conditioner of the present invention include both solids and liquids. By way of non-limiting example, the radiation source can be a radionuclide, such as I⁻¹²⁵, I⁻¹³¹, Yb⁻¹⁶⁹, Ir⁻¹⁹² as a solid source, I⁻¹²⁵ as a solid source, or other radionuclides that emit photons, beta particles, gamma radiation, or other therapeutic rays. The radioactive material can also be a fluid made from any solution of radionuclide(s), e.g., a solution of I⁻¹²⁵ or I⁻¹³¹, or a radioactive fluid can be produced using a slurry of a suitable fluid containing small particles of solid radionuclides, such as Au⁻¹⁹⁸, Y⁻⁹⁰. Moreover, the radionuclide(s) can be embodied in a gel or radioactive micro spheres.

There are many different medical and therapeutic applications in which heat is applied to the body. For instance, heat is applied to increase the body temperature when a person suffers from hypothermia. Heat is also applied to the body to establish certain temperature effects as they are known in the field of physical therapy and medicine (See for instance, U.S. Pat. No. 5,891,187). Other therapeutic applications involve administration of heat in combination with light, such as with photodynamic therapy (PDT) that encompasses a range of treatments that involve a combination of light and a photoactive chemical agent. In PDT, a light-activated drug is administered or applied to the patient, followed by illumination with intense light of a specific wavelength matched to the photosensitive qualities of the drug. Interaction between the drug and light causes destruction of the target tumor or lesion. Typically, a laser is used as the light source, however, other types of intense light sources may be used in certain dermatological applications. In some applications related to PDT, it is necessary to increase the temperature in a particular anatomical locus to approximately 41 degrees Celsius to locally release the content of a liposome encapsulating the photoactive chemical. Sometimes the temperature is accomplished by irradiating the laser light absorbing liposomes at the specific site via a laser beam. In some applications, (see e.g. U.S. Pat. No. 6,248,727) s a first laser beam is used to selectively heat tissue, including its blood vessels or sinuses, to a temperature of approximately 41 degrees Celsius without causing substantial physiological damage to that tissue or vasculature. A second laser beam is then used to suitably activate a tissue-reactive agent without causing substantial physiological damage to the vasculature in which it is disposed.

In U.S. Pat. No. 5,225,433 Dougherty et al. teaches a treatment in which a photosensitizing drug is injected into a subject. In treating humans or other mammals with the drug, light is irradiated on the tissue in such a position as to uniformly illuminate cancer tissue. Dougherty et al. then further teaches that heat is applied in the range of 40.5 to 45 degrees Celsius. The increase in temperature, accordingly to Dougherty et al., may be achieved by transmitting light: (1) some of which is near or in the infrared spectrum such as at 1060 nm wavelength from a Nd-Yag laser for heat with the light at 630 nm for interaction with the photosensitive drug; or (2) by microwaves such as the 2450 Mhz; or (3) by any other suitable means.

However, in any application where heat or light are applied to biological tissue during a medical or therapeutic application, irreversible unintended damage to the tissue is risked, and reversible changes are likely. Such treatments are cell conditioners within the scope of this invention.

Burns are also cell conditioners within the scope of this invention. In medicine, a burn is a type of injury caused by heat, electricity, chemicals, or radiation (an example of the latter is sunburn). Serious burns, especially if they cover large areas of the body, can cause death; any hint of burn injury to the lungs, for example through smoke inhalation, is a medical emergency. Chemical burns are usually caused by chemical compounds, such as sodium hydroxide (lye), silver nitrate, and more serious compounds (such as sulfuric acid). Most chemicals (but not all) that can cause moderate to severe chemical burns are strong acids or bases. Nitric acid is possibly one of the worst burn-causing chemicals, as an oxidizer. Hydrofluoric acid can eat down to the bone and its burns are often not immediately evident. Most chemicals that can cause moderate to severe chemical burns are called caustic. Electrical burns are generally symptoms of electrocution, being struck by lightning, being defibrillated or cardioverted without conductive gel, etc. The internal injuries sustained may be disproportionate to the size of the “burns” seen—as these are only the entry and exit wounds of the electrical current. Survival and outcome (scars, contractures, complications) of severe burn injuries is remarkably improved if the patient is treated in a specialized burn center/unit rather than a hospital. Scalding is a specific type of burning that is caused by hot fluids. Examples of common liquids that cause scalds are water and cooking oil. Steam is a common gas that causes scalds. The injury is usually regional and usually does not cause death. More damage can be caused if hot liquids can enter an orifice. However, deaths have occurred in more unusual circumstances, such as when people have accidentally broken a steam pipe. A cold burn is a kind of burn that arises when the skin is in contact with a low-temperature body. They can be caused by prolonged contact with moderately cold bodies (snow for instance) or brief contact with very cold bodies such as dry ice, liquid helium or liquid nitrogen, which are used in the process of wart removal. In such a case, the heat transfers from the skin and organs to the external cold body (as opposed to most other situations where the body causing the burn is hotter, and transfers the heat into the skin and organs). The effects are very similar to a “regular” burn. The remedy is also the same as for any burn: for a small wound keep the injured organ under a flow of comfortably temperatured water; the heat will then transfer slowly from the water to the organs and help the wound. Further treatment or treatment of more extended wound also as usual.

Cell conditioning also occurs by infection or exposure to a foreign species, such as a pathogen. An infection is the detrimental colonization of a host organism by a foreign species. In infection, the infecting organism seeks to utilize the host's resources in order to multiply (usually at the expense of the host). The infecting organism, or pathogen, interferes with the normal functioning of the host and can lead to chronic wounds, gangrene, loss of an infected limb, and even death. The host's response to infection or exposure may be inflammation. As used herein, pathogen includes without limitation, microscopic organisms, bacteria, parasites, intracellular parasites, fungi, viruses, prions, and viroids. Organisms that are normally non-pathogenic can become pathogenic under the right conditions, and even the most virulent organism requires certain circumstances to cause a compromising infection. Cell conditioning by infection can take place in vivo, ex vivo or in vitro, like any of the conditioning agents.

Another cellular conditioner within the scope of this invention is inflammation. Inflammation is the first response of the immune system to infection or irritation and may be referred to as the innate cascade. Inflammation is characterized by the following quintet: redness, heat, swelling, pain and dysfunction of the organs involved. Inflammation has two main components—cellular and exudative.

The exudative component involves the movement of fluid, usually containing many important proteins such as fibrin and immunoglobulins (antibodies). Blood vessels are dilated upstream of an infection (causing redness and heat) and constricted downstream while capillary permeability to the affected tissue is increased, resulting in a net loss of blood plasma into the tissue—giving rise to edema or swelling. The swelling distends the tissues, compresses nerve endings, and thus causes pain.

The cellular component involves the movement of white blood cells from blood vessels into the inflamed tissue. The white blood cells, or leukocytes, take on an important role in inflammation; they extravasate (filter out) from the capillaries into tissue, and act as phagocytes, picking up bacteria and cellular debris. They may also aid by walling off an infection and preventing its spread.

If inflammation of the affected site persists, released cytokines IL-1 and TNF will activate endothelial cells to upregulate receptors VCAM-1, ICAM-1, E-selectin, and L-selectin for various immune cells. Receptor upregulation increases extravasation of neutrophils, monocytes, activated T-helper and T-cytotoxic, and memory T and B cells to the infected site. These cytokines are all cell conditioners within the scope of this invention.

Neutrophils are characteristic of inflammation in the early stages—they are the first cells to appear in an infected area, and any section of recently inflamed (within a couple of days or so) tissue viewed under a microscope will appear packed with them. They are easily identified by their multilobed nuclei and granular cytoplasm and perform many important functions, including phagocytosis and the release of extracellular chemical messengers. Neutrophils only live for a couple days in these interstitial areas, so if the inflammation persists for a longer duration then they are gradually replaced by longer-lived monocytes.

Various leukocytes are involved in the initiation and maintenance of inflammation. These cells can be further stimulated to maintain inflammation through the action of adaptive cascade through lymphocytes: T cells, B cells, and antibodies. These inflammation cells are: a) mast cells that release histamine and prostaglandin in response to activation of stretch receptors, which is especially important in cases of trauma, and b) macrophages that release TNF-alpha, IL-1 in response to activation of toll-like receptors.

The outcome in a particular circumstance will be determined by the tissue in which the injury has occurred, and the injurious agent that is causing it. There are four possible results to inflammation.

The first of these is resolution, meaning the complete reconstitution of damaged tissue, which does not usually occur in the body. The second outcome is connective tissue scarring which comes through the wound healing response that commences within about twenty-four hours after inflammation in a wound first occurs. This response involves the formation of connective tissue to bridge the gap caused by injury, and the process of angiogenesis, the formation of new blood vessels, to provide nutrients to the newly formed tissue. Often healing cannot occur completely and a scar will form; for example after laceration to the skin, a connective tissue scar results that does not contain any specialized structures such as hair or sweat glands. The third outcome is abscess formation—primarily in infections by pyogenic bacteria.

The fourth result is ongoing or chronic inflammation. If the injurious agent continues, chronic inflammation will ensue. This process, marked by inflammation lasting many days, months or even years, may lead to the formation of a chronic wound. Chronic inflammation is characterized by a dominating presence of macrophages in the injured tissue, which extravasate via the same methods discussed above (ICAM-1 VCAM-1). These cells are powerful defensive agents of the body, but the toxins they release (including reactive oxygen species) are injurious to the organism's own tissues as well as invading agents. This is why chronic inflammation is almost always accompanied by tissue destruction. Finally, an abscess, or a collection of pus, can form in chronic inflammation.

When inflammation overwhelms the whole organism, systemic inflammatory response syndrome (SIRS) is diagnosed. When it is due to infection, the term sepsis is applied. Vasodilation and organ dysfunction are serious problems that may lead to septic shock and death.

Cellular conditioning also occurs with systemic inflammation. Although the processes involved are as described above, this form of inflammation is not confined to a particular tissue but involves the endothelium (lining of blood vessels) and many other organ systems. Systemic inflammation appears in a variety of disorders. High levels of several inflammation-related markers such as IL-6, IL-8, and TNF-alpha for example are associated with obesity. These levels are reduced in association with increased levels of anti-inflammatory molecules within weeks after patients begin a very low calorie diet. The role of systemic inflammation as a cause and/or result of insulin resistance and atherosclerosis is also being studied.

Inflammatory conditions that provide cellular conditioning include without limitation the following: Appendicitis, Arteritis, Arthritis, Blepharitis, Bronchiolitis, Bronchitis, Bursitis, Cervicitis, Cholangitis, Cholecystitis, Chorioamnionitis, Colitis, Conjunctivitis, Cystitis, Dacryoadenitis, Dermatitis, Dermatomyositis, Encephalitis, Endocarditis, Endometritis, Enteritis, Enterocolitis, Epicondylitis, Epididymitis, Fasciitis, Fibrositis, Gastritis, Gastroenteritis, Gingivitis, Hepatitis, Hidradenitis suppurativa, Ileitis, Iritis, Laryngitis, Mastitis, Meningitis, Myelitis, Myocarditis, Myositis, Nephritis, Omphalitis, Oophoritis, Orchitis, Osteitis, Otitis, Pancreatitis, Parotitis, Pericarditis, Peritonitis, Pharyngitis, Pleuritis, Phlebitis, Pneumonitis, Proctitis, Prostatitis, Pyelonephritis, Rhinitis, Salpingitis, Sinusitis, Stomatitis, Synovitis, Tendonitis, Tonsillitis, Uveitis, Vaginitis, Vasculitis, and Vulvitis.

Metabolic disorders are also cellular conditioners within the scope of this invention. A metabolic disorder affects the production of energy within individual human (or animal) cells. Most metabolic disorders are genetic, though a few are “acquired” as a result of diet, toxins, infections, etc. Genetic metabolic disorders are also known as inborn errors of metabolism. In general, the genetic metabolic disorders are caused by genetic defects that result in missing or improperly constructed enzymes necessary for some step in the metabolic process of the cell. The three largest classes of metabolic disorders are: a) Glycogen storage diseases (disorders affecting carbohydrate metabolism), b) Fatty oxidation disorders (disorders affecting the metabolism of fat components), and c) Mitochondrial disorders (disorders affecting the mitochondria which are the central “powerhouses” of the cells). A fourth class, the channelopathies (some of which cause periodic paralysis and/or malignant hyperthermia) could be considered to be metabolic disorders as well, though they are not always classified as such. These disorders affect the ion channels in the cell and organelle membranes, resulting in improper or inefficient transfer of ions through the membranes. There are also a number of other metabolic disorders (such as myoadenylate deaminase deficiency) that do not cleanly fit into any of the above classifications.

Another cellular conditioner is hypoxia, which is a pathological condition in which the body as a whole (generalized hypoxia) or region of the body (tissue hypoxia) is deprived of adequate oxygen supply. Hypoxia in which there is complete deprivation of oxygen supply is referred to as anoxia. Hypoxia is often associated with high altitudes, where it is called altitude sickness. Hypoxia can also occur while diving underwater, especially with closed-circuit rebreather systems that control the amount of oxygen in the air breathed in.

Generalized hypoxia may be due to low levels of oxygen in the blood (hypoxemia) or where tissues throughout the body are unable to utilize the oxygen supplied. Hypoxic hypoxia occurs when there is an inadequate supply of oxygen. This may be due to: a) Low partial pressure of atmospheric oxygen (e.g. at high altitudes or as may be caused during scuba diving), b) Inadequate pulmonary ventilation (e.g. in chronic obstructive pulmonary disease or respiratory arrest), c) Shunts in the pulmonary circulation or a right-to-left shunt in the heart. Shunts can be caused by collapsed alveoli that are still perfused or a block in ventilation to an area of the lung. Whatever the mechanism, blood meant for the pulmonary system is not ventilated and so no gas exchange occurs (the ventilation/perfusion ratio is zero). Standard existing shunts include the thebesian vessels that empty into the left ventricle and the bronchial circulation which supply the bronchi with oxygen.

Another example of generalized hypoxia is carbon monoxide poisoning that inhibits hemoglobins ability to bind oxygen. Generalized hypoxia also includes anemic hypoxia in which arterial oxygen pressure is normal, but total oxygen content of the blood is reduced. This may be due to: a) Reduced hemoglobin content in erythrocytes. (Since hemoglobin carries oxygen and carbon dioxide (and/or carbon monoxide), the quantity (volume) of oxygen carried is affected by how much hemoglobin is present in the red blood cells. For example iron deficiency anemia lowers hemoglobin levels in red blood cells and therefore hinders their carrying capacity.); b) Decreased hematocrit e.g. from blood loss (blood loss anemia).

Other examples of generalized hypoxia include hypemic hypoxia when there is an inability of the blood to carry oxygen, and histotoxic hypoxia, in which quantity of oxygen reaching the cells is normal, but the cells are unable to effectively use the oxygen.

Localized tissue hypoxia disorders include a) Ischemic, or stagnant hypoxia in which there is a local restriction in the flow of otherwise well-oxygenated blood. The oxygen supplied to the region of the body is then insufficient for its needs. Examples are cerebral ischemia to the brain and ischemic heart disease; b) Cerebral hypoxia in which the brain is deprived of oxygen despite normal blood flow; and c) Intrauterine hypoxia, which is an unchallenged cause of perinatal death.

The measurement of oxygen supply in hypoxia involves calculating its partial pressure by multiplying atmospheric pressure (e.g. 760 mmHg minus the 47 mm Hg of water vapor) by the gas's fraction in air (e.g., 713 mm Hg×21%=150 mm Hg). After mixing with expired CO2 in the lungs, oxygen diffuses down a pressure gradient to enter arterial blood around where its partial pressure is 100 mm Hg. Arterial blood flow delivers oxygen to the peripheral tissues, where it again diffuses down a pressure gradient into the cells and into their mitochondria. These bacterial like cytoplasmic structures strip hydrogen from fuels (glucose, fats and some amino acids) to burn with oxygen to form water. Released energy (originally from the sun and photosynthesis) is stored as ATP, to be later used for energy requiring metabolism. The fuel's carbon is oxidized to CO2, which diffuses down its partial pressure gradient out of the cells into venous blood to finally be exhaled by the lungs. Experimentally, oxygen diffusion becomes rate limiting (and lethal) when arterial oxygen partial pressure falls to 40 mm Hg or below.

If oxygen delivery to cells is insufficient for the demand (hypoxia), hydrogen will be shifted to pyruvic acid converting it to lactic acid. This temporary measure (anaerobic metabolism) allows small amounts of energy to be produced. Lactic acid build-up in tissues and blood is a sign of inadequate mitochondrial oxygenation, which may be due to hypoxemia, poor blood flow (e.g. shock) or a combination of both. If severe or prolonged it could lead to cell death.

Another cellular conditioner is electric charge, which may be painful and can be lethal. The level of voltage is not a direct guide to the level of injury or danger of death. A small shock from static electricity may contain thousands of volts but has very little current behind it due to high internal resistance. Physiological effects and damage are generally determined by current and duration of the shock. Even a low voltage causing a current of extended duration can be fatal. Ohm's Law directly correlates voltage and current for a given resistance; thus, for a particular path through the body under a particular set of conditions, a higher voltage will produce a higher current flow. With sufficiently high current there can be a muscular spasm that causes the affected person to grip and be unable to release from the current source. The maximum current that can cause the flexors of the arm to contract but that allows a person to release his hand from the current's source is termed the let-go current. For direct current, the let-go current is about 75 mA for a 70-kg man. For alternating current, the let go current is about 15 mA, dependent on muscle mass.

The perception of electric charge can be different depending on the voltage, duration, current, path taken, frequency, etc. Current entering the hand has a threshold of perception of about 5 to 10 milliamperes (mA) for DC and about 1 to 10 mA for AC at 60 Hz. Shock perception declines with increasing frequency, ultimately disappearing at frequencies above 15-20 kHz.

A physiological effect of electric charge is tissue heating due to resistance, which can cause extensive and deep burns. High-voltage (>500 to 1000 V) shocks tend to cause internal burns due to the large energy (which is proportional to the square of the voltage) available from the source. Damage due to current is through tissue heating.

Ventricular fibrillation is another physiological effect of electric charge. A low-voltage (110 to 220 V), 60-Hz AC current traveling through the chest for a fraction of a second may induce ventricular fibrillation at currents as low as 60 mA. With DC, 300 to 500 mA is required. If the current has a direct pathway to the heart (e.g., via a cardiac catheter or other electrodes), a much lower current of less than 1 mA, (AC or DC) can cause fibrillation. Fibrillations are usually lethal because all the heart muscle cells move independently. Above 200 mA, muscle contractions are so strong that the heart muscles cannot move at all. Electric current can also cause interference with nervous control, especially over the heart and lungs.

Other issues affecting damage from electric charge are frequency, which is an issue in causing cardiac arrest or muscular spasms, and pathway—if the current passes through the chest or head there is an increased chance of death. From a mains circuit the damage is more likely to be internal, leading to cardiac arrest. Effects of exposure to alternating current and direct current may be different. It is believed that human lethality is most common with AC current at 100-250 volts, as lower voltages can fail to overcome body resistance while with higher voltages the victim's muscular contractions are often severe enough to cause them to recoil (although there will be considerable burn damage). However, death has occurred from supplies as low as 32 volts. Electrical discharge from lightning tends to travel over the surface of the body causing burns and may cause respiratory arrest.

Electric charge can also be used as a medical therapy, under carefully engineered conditions, such as a) as a (disputed) psychiatric therapy for mental illness (Electroconvulsive therapy), and b) as a treatment for fibrillation or irregular heart rhythms.

As used in this invention, electrical charge also includes the electric charge produced by normal biological processes, such as for example the energy released during respiration and the production or utilization of ATP, a molecule that fuels chemical reactions in the cell. In certain settings, free radicals generated during respiration are associated with cell damage and in other settings are involved with proteins (transcription factors) that are involved in controlling gene activity and adapting cells to changing circumstances. These interactions serve to condition a cell for purposes of this invention.

Another cellular condition is oxidative stress, which refers to damage to animal or plant cells (and thereby the organs and tissues composed of those cells) caused by reactive oxygen species, which include (but are not limited to) superoxide, singlet oxygen, peroxynitrite or hydrogen peroxide. Oxidative stress is an imbalance between pro-oxidants and anti-oxidants, with the former prevailing. Superoxide is produced deleteriously by 1-electron transfers in the mitochondrial electron transfer chain. Other enzymes capable of producing superoxide are xanthine oxidase, NADPH oxidases and cytochrome P450(s). Hydrogen peroxide is produced by a wide variety of enzymes including monoxygenases and oxidases. Reactive oxygen species may also play a role in cell signaling. Oxidative stress is known to contribute to tissue injury following irradiation and hyperoxia and is thought to be a cause of neurodegenerative diseases including Lou Gehrig's disease (aka MND or ALS), Parkinson's disease, Alzheimer's disease and Huntington's disease. Oxidative stress is thought to be linked to certain cardiovascular disease, since oxidation of LDL in the endothelium is a precursor to plaque formation.

Metal catalysts are involved in oxidative stress. Metals such as iron, copper, chromium, vanadium and cobalt are capable of redox cycling in which a single electron may be accepted or donated by the metal. This action catalyzes reactions that produce reactive radicals and can produce reactive oxygen species such as hydroxyl radical in reactions like Fenton's reaction. The hydroxyl radical then can lead to modifications of amino acids (e.g. meta-tyrosine and ortho-tyrosine formation from phenylalanine), carbohydrates, initiate lipid peroxidation. Most enzymes that produce reactive oxygen species contain one of these metals. The presence of such metals in biological systems in an unsequestered form (not in an enzyme or other protein) can significantly increase the level of oxidative stress.

Other cellular conditioners include growth factors and other factors that are biologically active proteins or factors that are secreted by cells and can alter cell membranes (e.g. proteases) yet are not growth factors. A growth factor is a protein that acts as a signaling molecule between cells (like cytokines and hormones) that attaches to specific receptors on the surface of a target cell and promotes differentiation and maturation of these cells. The term growth factor is sometimes used to refer to cytokines. Growth factor signifies a positive effect on cell growth and cellular differentiation, but cytokine is a neutral term in regards to what it is being signaled. In this sense, some cytokines can be growth factors such as G-CSF and GM-CSF as listed below. However some cytokines are actually used as “death” signals, such as the FAS ligand, which causes target lymphocytes to commit a form of suicide known as programmed cell death or apoptosis.

Individual growth factor proteins tend to occur as members of larger families of structurally and evolutionarily related proteins. There are many growth factor families such as TGF-beta (transforming growth factor), BMP (bone morphogenic protein), neurotrophins (NGF, BDNF, and NT3), fibroblast growth factor (FGF), etc. Several well known growth factors are: granulocyte-colony stimulating factor (G-CSF), granulocyte-macrophage colony stimulating factor (GM-CSF), nerve growth factor (NGF), neurotrophins, platelet-derived growth factor (PDGF), erythropoietin (EPO), thrombopoietin (TPO), myostatin (GDF-8), Growth Differentiation factor-9 (GDF9), and basic fibroblast growth factor (bFGF or FGF2).

Growth factors have been used in treatment of a variety of hematologic and oncologic diseases, including without limitation: neutropenia, myelodysplastic syndrome (MDS), leukemias, aplastic anemia, and bone marrow transplantation. Cellular conditioning occurs when a cell is exposed to a new growth factor or to a familiar growth factor at an increased level of exposure. Cellular conditioning also occurs when a cell that has been accustomed to a particular level of a growth factor in its environment is deprived of that growth factor in total or in part.

Cellular conditioning is also provided by hormones. A hormone is a chemical messenger from one cell (or group of cells) to another. All multicellular organisms produce hormones, including plants. Hormones are produced by nearly every organ system and tissue type in an animal body. Hormone molecules are secreted directly into the bloodstream; some hormones, called ectohormones, aren't secreted into the blood stream, they move by circulation or diffusion to their target cells, which may be nearby cells (paracrine action) in the same tissue or cells of a distant organ of the body. The function of hormones is to serve as a signal to the target cells; the action of hormones is determined by the pattern of secretion and the signal transduction of the receiving tissue.

Hormone actions vary widely, but can include stimulation or inhibition of growth, induction or suppression of apoptosis (programmed cell death), activation or inhibition of the immune system, regulating metabolism and preparation for a new activity (e.g., fighting, fleeing, mating) or phase of life (e.g., puberty, caring for offspring, menopause, male neropause). In many cases, one hormone may regulate the production and release of other hormones. Many of the responses to hormone signals can be described as serving to regulate metabolic activity of an organ or tissue. Hormones also control the reproductive cycle of virtually all multicellular organisms.

Most cells are capable of producing one or more, sometimes many, molecules that signal other cells to alter their growth, function, or metabolism. The classical endocrine glands and their hormone products are specialized to serve regulation on the overall organism level, but can often be used in other ways or only on the tissue level. The rate of production of a hormone is often regulated by a homeostatic control system, generally by negative feedback. Homeostatic regulation of hormones depends, apart from production, on the metabolism and excretion of hormones. Hormone secretion can be stimulated and inhibited by: a) Other hormones (stimulating- or releasing-hormones); b) Plasma concentrations of ions or nutrients, as well as binding globulins; c) Neurons and mental activity; and d) Environmental changes, e.g., of light or temperature.

One special group of hormones is trophic hormones that stimulate the hormone production of other endocrine glands. For example: thyroid-stimulating hormone (TSH) causes growth and increased activity of another endocrine gland—the thyroid—hence increasing the output of thyroid hormones. Another class of hormones is that of the “Hunger Hormones”—ghrelin, orexin and PYY 3-36—and “Satiety hormones”—e.g., leptin, obestatin.

Vertebrate hormones fall into four chemical classes: 1) Amine-derived hormones are derivatives of the amino acids tyrosine and tryptophan. Examples are catecholamines and thyroxine; 2) Peptide hormones that consist of chains of amino acids. Examples of small peptide hormones are TRH and vasopressin. Examples of protein hormones include insulin and growth hormone; 3) Steroid hormones derived from cholesterol. The adrenal cortex and the gonads are primary sources. Examples of steroid hormones are testosterone and cortisol. Sterol hormones such as calcitriol are a homologous system; and 4) Lipid and phospholipid hormones, derived from lipids such as linoleic acid and phospholipids such as arachidonic acid. Steroid hormones include the eicosanoids, which includes the prostaglandins. All maybe used as conditioning agents.

Many hormones are used as medication, such as for example estrogens and progestagens (in the contraceptive pill and as HRT), thyroxine (as levothyroxine, for hypothyroidism) and steroids (for autoimmune diseases and several respiratory disorders). Insulin is used by many diabetics. Local preparations for use in otolaryngology often contain pharmacologic equivalents of adrenaline, while steroid and vitamin D creams are used extensively in dermatological practice.

Suitable cellular conditioners also include anti-hormonal agents that act to regulate or inhibit hormone action on tumors such as anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors including vorozole (RIVISOR®), megestrol acetate (MEGASE®), fadrozole, imidazole; lutenizing hormone-releasing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; testolactone; and pharmaceutically acceptable salts, acids or derivatives of any of the above; as well as combinations of two or more of the above.

A “pharmacologic dose” of a hormone is a medical usage referring to an amount of a hormone far greater than naturally occurs in a healthy body. The effects of pharmacologic doses of hormones may be different from responses to naturally occurring amounts and may be therapeutically useful. An example is the ability of pharmacologic doses of glucocorticoid to suppress inflammation.

Important human hormones include the amine-derived hormones, are the catecholamines (adrenaline/epinephrine, dopamine, noradrenaline/norepinephrine), tryptophan derivatives (melatonin, serotonin), and tyrosine derivatives (thyroxine, triiodothyronine).

Peptide hormones include: antimullerian hormone (AMH, also mullerian inhibiting factor or hormone), adiponectin (also Acrp30), adrenocorticotropic hormone (ACTH, also corticotropin), angiotensinogen and angiotensin, antidiuretic hormone (ADH, also vasopressin, arginine vasopressin, AVP), atrial-natriuretic peptide (ANP, also atriopeptin), calcitonin, cholecystokinin (CCK), corticotropin-releasing hormone (CRH), erythropoietin (EPO), follicle-stimulating hormone (FSH), gastrin, ghrelin, glucagons, gonadotropin-releasing hormone (GnRH), growth hormone-releasing hormone (GHRH), human chorionic gonadotropin (hCG), growth hormone (GH, or hGH), inhibin, insulin, insulin-like growth factor (IGF, also somatomedin), leptin, luteinizing hormone (LH), melanocyte stimulating hormone (MSH or α-MSH), neuropeptide Y, oxytocin, parathyroid hormone (PTH), prolactin (PRL), relaxin, secretin, somatostatin, thrombopoietin, thyroid-stimulating hormone (TSH), and thyrotropin-releasing hormone (TRH).

Steroid hormones include: Glucocorticoids (cortisol), Miferalocorticoids (aldosterone), Sex steroids including Androgens (testosterone, dehydroepiandrosterone (DHEA), dehydroepiandrosterone sulfate (DHEAS), androstenedione, and dihydrotestosterone (DHT)), Estorogens (estradiol), Progestagens (progesterone, Progestins). Sterol hormones include for example, Vitamin D derivatives such as calcitriol. Lipid and phospholipid hormones (eicosanoids) include prostaglandins, leukotrienes, prostacyclin, and thromboxane.

In some embodiments, cellular conditioning occurs when a cell is exposed to a new hormone or to a familiar hormone at an increased level of exposure. Cellular conditioning also occurs when a cell that has been accustomed to a particular level of a hormone in its environment is deprived of that hormone in total or in part.

Cellular conditioning is also involved in cell adhesion processes that generally involve protein molecules at the surface of cells. Transmembrane cell adhesion proteins extend across the cell surface membrane and typically have domains that extend into both the extracellular space and the intracellular space. The extracellular domain of a cell adhesion protein can bind to other molecules that might be either on the surface of an adjacent cell (cell-to-cell adhesion) or part of the extracellular matrix (cell-to-ECM adhesion). The molecule that a cell adhesion protein binds to is called its ligand. There are families of cell adhesion proteins that can be characterized in terms of the structure of the adhesion proteins and their ligands. Adhesion between two copies of the same adhesion protein is called “homophilic” binding. Adhesion between an adhesion protein and some other molecule is “heterophilic” binding. Major Cell Adhesion Protein Families include the Selections that bind to Carbohydrates (heterophilic), the Integrins that bind to Extracellular matrix proteins (heterophilic) and to Immunoglobulin superfamily proteins (heterophilic), Immunoglobulin superfamily proteins that bind to Integrins (heterophilic) and to other Immunoglobulin superfamily proteins (homophilic) and the Cadherins that bind to Cadherins (homophilic).

Cell-cell interactions also include cytoskeletal interactions, where for example the intracellular domain of a cell adhesion protein binds to protein components of the cell's cytoskeleton. This allows for very tight adhesion. Without attachment to the cytoskeleton, a cell adhesion protein that is tightly bound to a ligand would be in danger of being hydrolyzed by extracellular hydrolytic enzymes. This will rip out the adhesion protein from the fragile cell membrane. Sometimes the connection between the cell adhesion proteins and the cytoskeleton is not direct. For example, cadherin cell adhesion proteins are typically coupled to the cytoskeleton by way of special linking proteins called “catenins”.

Cell adhesion proteins are important for the normal functioning of living organisms. Cell adhesion proteins hold together the components of solid tissues. They are also important for the function of migratory cells like white blood cells. Regulation of cell adhesion proteins is important during embryonic development for the process of morphogenesis. Some people have “blistering diseases” that result from inherited molecular defects in genes for adhesion proteins. Some cancers involve mutations in genes for adhesion proteins that result in abnormal cell-to-cell interactions and tumor growth. Cell adhesion proteins are also important for interactions that allow viruses and bacteria to cause damage to humans. Cell adhesion proteins hold synapses together and the regulation of synaptic adhesion is involved in learning and memory. In Alzheimer's disease there is abnormal regulation of synaptic cell adhesion.

Cellular conditioning occurs when a cell is exposed to a new cell adhesion protein or to a familiar cell adhesion protein at an increased level of exposure. Cellular conditioning also occurs when a cell that has been accustomed to a particular level of a cell adhesion protein in its environment is deprived of that cell adhesion protein in total or in part.

Cellular conditioning is also caused for example and without limitation by changing levels of exposure to other soluble factors, and to cell and tissue damage from other causes.

Chemotherapy may be given prior to surgery to shrink a large tumor prior to a surgical procedure to remove it, after surgery or radiation therapy to prevent the growth of any remaining cancer cells in the body. Examples of chemotherapeutic agents suitable as cell conditioners according to the teachings of this invention include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheanmcin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethyla-mine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL™, Bristol-Myers Squibb Oncology, Princeton, N.J.) and docetaxel (TAXOTERE™, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

Exemplary therapeutic agents include but are not limited to agents as described above, radiation therapy, hormone antagonists, hormones and their releasing factors, thyroid and antithyroid drugs, estrogens and progestins, androgens, adrenocorticotropic hormone; adrenocortical steroids and their synthetic analogs; inhibitors of the synthesis and actions of adrenocortical hormones, insulin, oral hypoglycemic agents, and the pharmacology of the endocrine pancreas, agents affecting calcification and bone turnover: calcium, phosphate, parathyroid hormone, vitamin D, calcitonin, vitamins such as water-soluble vitamins, vitamin B complex, ascorbic acid, fat-soluble vitamins, vitamins A, K, and E, growth factors, cytokines, chemokines, muscarinic receptor agonists and antagonists; anticholinesterase agents; agents acting at the neuromuscular junction and/or autonomic ganglia; catecholamines, sympathomnimetic drugs, and adrenergic receptor agonists or antagonists; and 5-hydroxytryptamine (5-HT, serotonin) receptor agonists and antagonists.

Therapeutic agents can also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, β-adrenergic agonists, ipratropium, glucocorticoids, methylxanthines, sodium channel blockers, opioid receptor agonists, calcium channel blockers, membrane stabilizers and leukotriene inhibitors.

Other therapeutic agents contemplated herein include diuretics, vasopressin, agents affecting the renal conservation of water, rennin, angiotensin, agents useful in the treatment of myocardial ischemia, anti-hypertensive agents, angiotensin converting enzyme inhibitors, β-adrenergic receptor antagonists, agents for the treatment of hypercholesterolemia, and agents for the treatment of dyslipidemia.

Additional therapeutic agents contemplated include drugs used for control of gastric acidity, agents for the treatment of peptic ulcers, agents for the treatment of gastroesophageal reflux disease, prokinetic agents, antiemetics, agents used in irritable bowel syndrome, agents used for diarrhea, agents used for constipation, agents used for inflammatory bowel disease, agents used for biliary disease, agents used for pancreatic disease. Therapeutic agents used to treat protozoan infections, drugs used to treat Malaria, Amebiasis, Giardiasis, Trichomoniasis, Trypanosomiasis, and/or Leishmaniasis, and/or drugs used in the chemotherapy of helminthiasis. Other therapeutic agents include antimicrobial agents, sulfonamides, trimethoprim-sulfamethoxazole quinolones, and agents for urinary tract infections, penicillins, cephalosporins, and other, b-Lactam antibiotics, an agent comprising an aminoglycoside, protein synthesis inhibitors, drugs used in the chemotherapy of tuberculosis, mycobacterium avium complex disease, and leprosy, antifungal agents, antiviral agents including nonretroviral agents and antiretroviral agents.

Moreover, therapeutic agents used for immunomodulation, such as immunomodulators, immunosuppressive agents, tolerogens, and immunostimulants are contemplated by the methods herein. In addition, therapeutic agents acting on the blood and the blood-forming organs, hematopoietic agents, growth factors, minerals, and vitamins, anticoagulant, thrombolytic, and antiplatelet drugs.

Further therapeutic agents may be found in Goodman and Gilman's “The Pharmacological Basis of Therapeutics” Tenth Edition edited by Hardman, Limbird and Gilman or the Physician's Desk Reference, both of which are incorporated herein by reference in their entirety.

a. Determination of Dosing

In certain aspects, the present invention relates to the determination of suitable dose ranges for any of the previously mentioned cell conditioners or cell conditioning agents. Generally, the suitable dose range for a cell conditioner or cell conditioning agent is largely determined by the type of cell and the conditioner/ conditioning agent being used. In some embodiments, the cell conditioner or conditioning agent should be used at a dose where it allows the conditioned cells to present at least one target antigen, as compared to a cell that has not been conditioned.

In certain embodiments, the precautions should be taken to determine the dosage of the cellular conditioner to ensure cell viability of the subject conditioned cells. In such cases, a dose response of the cell conditioner or conditioning agent may be employed in order to determine the desired or optimal dose. One skilled in the art would readily recognize that the desired or optimal dose of a cell conditioner or conditioning agent would vary depending on the desired effect of using such cell conditioner or conditioning agent. The time of treatment of the subject of the cell conditioner will also vary depending on the conditioning agent and the desired effect. In some embodiments, the cell conditioner or conditioning agent may have a deleterious effect on the subject cell to be conditioned. In such cases, a dose response of the cell conditioner or conditioning agent to determine the maximal inhibitory concentration may be used. The optimal or desired dose of the cell conditioner or conditioning agent may be then chosen based on the results of such a dose response.

In one embodiment where the subject conditioned cells is to be used as a tool for generating antigen modulators specific for target cell surface antigens, it is important to ensure that the subject conditioned cells be viable with intact cellular membranes. In such a case, the dose of the cell conditioner/conditioning agent used would have to allow the conditioned cells to present at least one target antigen, as compared to a cell that has not been conditioned, yet allow for cell viability and an intact cellular membrane. Cell viability and membrane integrity can be assessed using several different methods that are well known in the art and include, but are not limited to, propidium iodide or trypan blue staining. Of course, membrane fragments of such conditioned cells may also be used to generate antigen modulators.

In certain embodiments, cells tested with a dose response of the desired cell conditioner or conditioning agent can be incorporated into a cell array for screening antigen modifiers for target antigen expression at an optimal dose of cell conditioner or conditioning agent. In other embodiments, arrays of conditioned cells, or cell membrane fragments may be made, with the array defined by me or more variables, including but not limited to conditioning agents concentration, dosage, combination w/one or more other agents, timing or duration of administration or exposure, etc. Many such arrays are known in the art and may be suitable. By way of example, the array described in U.S. Pat. No. 6,406,840 can incorporate cells/cell lines that have been conditioned with various cell conditioners or conditioning agents. Antigen modifiers, such as antibodies, can be screened for IHC reactivity on the array of conditioned cells to determine an optimal conditioning dose and cell type/line to be used in the characterization of that antigen modifier.

### III. “Conditioned” Antigens

The subject conditioned cell presents at least one altered antigen, as compared to a cell that has not been conditioned. Target antigens include new, enhanced, activated or altered epitopes. Representative antigens can be cell surface antigens, cytoplasmic, chaperone, or nuclear antigens. In a preferred embodiment, representative antigens comprise cell surface antigens.

The conditioned antigens can be classified into one or more of the following: proteins, peptides, carbohydrates, lipids, hydrophilic phosphorous molecules, nucleic acids, or any combinations thereof. Representative antigens can be produced in the conditioned cell by e.g., mRNA up-regulation, differential RNA splicing, post-transcriptional events, post-translational modifications, mutations introduced during the transcription or the translation process, extracellular protease exposure, novel hetero-dimer formation, altered glycosylation, altered phosphorylation, altered acetylation, altered methylation, altered biotinylation, altered glutamylation, altered glycylation, altered isoprenylation, altered lipoylation, altered phosphopantetheinylation, altered sulfation, altered ISGylation, altered SUMOylation, altered ubiquitination, altered citrullination, altered deamidation, altered disulfide bridges, altered proteolytic cleavage, altered translocation, changes in protein turnover (e.g. accumulation of protein in cell membrane), protein aggregation, oxidation, lipid aggregation, altered conformation, and biochemical changes in cell membrane.

Representative antigens of a conditioned cell may vary according to the type or kind of conditioning, duration, interval of administration and dose. In preferred embodiments, representative antigens of a conditioned cell include epitopes that are quantitatively or qualitatively modulated in the cell.

In another embodiment, representative antigens of a conditioned cell include epitopes from proteins that are newly expressed or have been upregulated at the protein level by the cellular conditioning.

### IV. Antigen Modulators

The subject conditioned cells provide a tool for generating antagonists, agonists and other antigen modulators specific for the target antigens of this invention. Accordingly, one aspect of the invention involves the generation of new modulators that are specific for antigens that are presented by a conditioned cell. In a preferred embodiment, the modulators are specific for cell surface antigens that are altered or expressed in a conditioned cell to a greater or lesser extent than in a cell that has not been exposed to the cell conditioning.

A modulator (agonist or antagonist) can be, without limitation, a biological or chemical compound such as a simple or complex organic or inorganic molecule, a peptide, a protein (e.g. antibody) or a polynucleotide (e.g. anti-sense). A vast array of compounds can be synthesized, for example polymers, such as polypeptides and polynucleotides, and synthetic organic compounds based on various core structures. In addition, various natural sources can provide compounds for screening, such as plant or animal extracts, and the like. It should be understood, although not always explicitly stated that a modulator can be used alone or in combination with another modulator, having the same or different biological activity.

A preferred class of modulators specific for an antigen on a conditioned cell is antibodies or antibody-like molecules including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the antigen that recognizes its receptor but imparts no effect, thereby competitively inhibiting the action of the antigen.

Another preferred class of modulators are small interfering RNA (siRNA)), sometimes known as short interfering RNA or silencing RNA, that are a class of 20-25 nucleotide-long RNA molecules that play a variety of roles in biology. SiRNA is involved in the RNA interference pathway (RNAi) where the siRNA interferes with the expression of a specific gene. In addition to their role in the RNAi pathway, siRNAs also act in RNAi-related pathways, e.g. as an antiviral mechanism or in shaping the chromatin structure of a genome.

Other modulators may be an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. The antisense RNA or DNA described above can also be delivered to cells such that the antisense RNA or DNA may be expressed in vivo to inhibit production of the target polypeptide. When antisense DNA is used, oligodeoxyribonucleotides derived from the translation-initiation site, e.g., between about −10 and +10 positions of the target gene nucleotide sequence, are preferred.

Antisense RNA or DNA molecules are generally at least about 5 bases in length, about 10 bases in length, about 15 bases in length, about 20 bases in length, about 25 bases in length, about 30 bases in length, about 35 bases in length, about 40 bases in length, about 45 bases in length, about 50 bases in length, about 55 bases in length, about 60 bases in length, about 65 bases in length, about 70 bases in length, about 75 bases in length, about 80 bases in length, about 85 bases in length, about 90 bases in length, about 95 bases in length, about 100 bases in length, or more.

Contemplated modulators include small molecules that bind to the active site, the receptor-binding site, or growth factor or other relevant binding site of an antigen identified herein, thereby blocking its normal biological activity. Examples of small molecules include, but are not limited to, small peptides or peptide-like molecules, preferably soluble peptides, and synthetic non-peptidyl organic or inorganic compounds. These small molecules can be identified by screening techniques well known for those skilled in the art.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques.

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex.

It is contemplated in the methods of the invention that a modulator can be generated as described below taking into account these variables. For example, some modulators bind antigens produced at a low dose of the cellular conditioner, while other modulators bind antigens produced at a higher dose of the cellular conditioner. In another example, some modulators bind early antigens produced at an early time point during cellular conditioning, while other modulators bind late antigens produced at a later time point during cellular conditioning. It is also contemplated in the methods of the invention to generate modulators that bind antigens produced during combination treatment with two or more cellular conditioning.

Computer modeling and searching technologies permit identification of compounds, or the improvement of already identified compounds, that can modulate antigen expression or activity. Having identified such a compound or composition as either the antigen or a target antigen modulator, the binding sites or regions are identified. Such binding sites might typically be the antigen-binding site or the binding site of the antigen modulator partner. The binding site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the binding site by finding where on the factor the complexed ligand is found.

Next, the three dimensional geometric structure of the binding site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the binding site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the binding region(s) of the target antigen or antigen modulator, either experimentally, by modeling, or by a combination, candidate modulating compounds can be identified by searching databases containing compounds along with information on their molecular structure. Such a search seeks compounds having structures that match the determined binding site structure and that interact with the groups defining the site(s) Such a search can be manual, but is preferably computer assisted. These compounds found from this search are potential antigen modulators.

Alternatively, these methods can be used to identify improved modulating compounds from an already known modulating compound or ligand. The composition of the known compound can be modified and the structural effects of modification can be determined using the experimental and computer-modeling methods described above applied to the new composition. The altered structure is then compared to the binding site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the binding sites of the differentially expressed genes, proteins, epitopes and/or target antigens of this invention and related transduction and transcription factors will be apparent to those of skill in the art.

Examples of molecular modeling systems are the CHARMm and QUANTA programs (Polygen Corporation, Waltham, Mass.). CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen et al., 1988, Acta Pharmaceutical Fennica 97:159-166; Ripka, New Scientist 54-57 (Jun. 16, 1988); McKinaly and Rossmann, 1989, Annu. Rev. Pharmacol. Toxiciol. 29:111-122; Perry and Davies, OSAR: Quantitative Structure—Activity Relationships in Drug Design pp. 189-193 (Alan R. Liss, Inc. 1989); Lewis and Dean, 1989, Proc. R. Soc. Lond. 236:125-140 and 141-162; and, with respect to a model receptor for nucleic acid components, Askew et al., 1989, J. Am. Chem. Soc. 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds that could alter binding, one could also screen libraries of known compounds, including natural products or synthetic chemicals, and biologically active materials, including proteins, for compounds that are modulators, inhibitors or activators.

a. Antibodies

Methods of preparing and/or selecting antibodies are known to the skilled artisan. According to the teachings herein, conditioned cells are used as immunogens to elicit an immune response in a recipient host animal. The route and schedule of immunization of the host animal are generally in keeping with established and conventional techniques for antibody stimulation and production.

While a mouse is usually employed as the test mode, it is contemplated that any mammalian subject including humans or antibody producing cells therefrom can be manipulated according to the processes of this invention to serve as the basis for production of mammalian, including human, hybridoma cell lines. Typically, the host animal is inoculated with an immunogenic amount of the cells, cell extracts, or protein preparations that contain the immunogen of interest, and then boosted with similar amounts of the immunizing agent. In an alternative, cells grown on non-biological membrane matrix are surgically implanted into the host animal, typically intraperitoneally. Lymphoid cells, preferably spleen lymphoid cells from the host, are collected a few days after the final boost and a cell suspension is prepared therefrom for use in the fusion.

The immunogen can be, but is not limited to, primary cells, cultured cell lines, cancerous cells, nucleic acids, or tissue. Methods for isolating and culturing suitable cells are detailed in Example 1. Cells used for immunization may be cultured for a period of time (at least 24 hours) prior to their use as an immunogen. The cells may be cultured in serum free conditions. In one embodiment, cells are isolated and grown in culture without the presence of serum biomolecules. Serum is an extremely complex mixture of many small and large biomolecules with undefined activities. For most cells, serum is not the physiological fluid which they contact in the original tissue environment from which they are derived. In vivo, a cell would be exposed to the equivalent of serum only under special circumstances involving tissue injury and blood coagulation. In vitro, various hormones and growth factors present in the serum can stimulate excessive growth, cellular changes, and/or terminal differentiation, accompanied by an altered expression of cell surface antigens, secretory, cytosolic or nuclear proteins. The complex mixture of serum factors may also inhibit growth and/or differentiation of a particular cell type, resulting in a change in cell morphology, physiology and/or viability. Moreover, numerous kinds of serum biomolecules are known to adhere to the cell surfaces. These biomolecules include, but are not limited to, transfer proteins (e.g., albumin), attachment and spreading factors (e.g., collagen and fibronectin), and various kinds of serum lipids. Adsorption of these exogenous molecules to the cell surface not only results in the generation of antibodies cross-reacting with molecules that are not present on the cell surface of a particular cell type, but can also mask presentation of the native antigens present on the cell surface during the antibody generation process.

Cells or membrane fragments may be used as immunogens by themselves or in combination with a non-denaturing adjuvant, such as Ribi. In general, cells may be kept intact and preferably viable when used as immunogens, although intact membrane fragments may be used. Intact cells may allow antigens to be better detected than ruptured cells by the immunized animal. Use of denaturing or harsh adjuvants, e.g., Freund's adjuvant, may rupture the cells and therefore is discouraged. The immunogen may be administered multiple times at periodic intervals such as, bi-weekly, or weekly, or may be administered in such a way as to maintain viability in the animal (e.g., in a tissue recombinant). Example 2 describes methods used to generate suitable antibodies for use in the practice of this invention.

In one embodiment, monoclonal antibodies that bind to a target antigen are obtained by using host cells that over-express that target antigen as an immunogen. Such cells include, by way of example and not by limitation, human fetal cells and human cancer cells.

Antibodies are produced from an animal immunized accordingly to standard methods in the art. For production polyclonal antibodies, by one or more injections of the subject immunogens can be injected alone or if desired, with an adjuvant. Typically, the immunizing agent and/or adjuvant will be injected in the mammal by multiple subcutaneous or intraperitoneal injections. In some embodiments, it may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A, synthetic trehalose dicorynomycolate). In preferred embodiments, the immunogen is administered without adjuvant. The immunization protocol may be selected by one skilled in the art without undue experimentation.

To monitor the antibody response, a small biological sample (e.g., blood) may be obtained from the animal and tested for antibody titer against the immunogen. The spleen and/or several large lymph nodes can be removed and dissociated into single cells. If desired, the spleen cells may be screened (after removal of non-specifically adherent cells) by applying a cell suspension to a plate or to a well coated with the antigen. B-cells, expressing membrane-bound immunoglobulin specific for the antigen, will bind to the plate, and are not rinsed away with the rest of the suspension. Resulting B-cells, or all dissociated spleen cells, can then be fused with myeloma cells (e.g., X63-Ag8.653 and those from the Salk Institute, Cell Distribution Center, San Diego, Calif.). Polyethylene glycol (PEG) may be used to fuse spleen or lymphocytes with myeloma cells to form a hybridoma. The hybridoma is then cultured in a selective medium (e.g., hypoxanthine, aminopterin, thymidine medium, otherwise known as “HAT medium”), however examples of different media that can be used for culturing hybridomas that secrete monoclonal antibodies are known in the art. The resulting hybridomas are then plated by limiting dilution, and are assayed for the production of antibodies that bind specifically to the immunogen (e.g., surface of the human fetal cells, surface of cancer cell lines, target antigen, etc.) using FACS or immunohistochemistry (IHC screening). The selected monoclonal antibody-secreting hybridomas are then cultured either in vitro (e.g., in tissue culture bottles or hollow fiber reactors), or in vivo (e.g., as ascites in mice). As another alternative to the cell fusion technique, EBV immortalized B cells are used to produce the monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas of the present invention encompass all derivatives, progeny cells of the parent hybridomas that produce monoclonal antibodies specific for antigens representative of the type of cells used for immunization.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present can be removed, for example, by running the preparation over adsorbents made of the immunogen attached to a solid phase and eluting or releasing the desired antibodies off the immunogen.

In another alternative, the monoclonal antibody can be sequenced and produced recombinantly by any means known in the art (e.g., humanization, use of transgenic mice to produce fully human antibodies, phage display technology, etc.). The monoclonal antibodies may be made by recombinant DNA methods such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. Prokaryotic hosts, such as E. coli are also suitable for the recombinant production of the antibodies herein. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The monoclonal antibodies may also be made by in vitro immunization/activation of non-transformed human B cells. The procedures of in vitro immunization of B cells are generally in keeping with established and conventional techniques of B cell activation. The process involves contacting a population of non-transformed human B cells with a desired antigen under conditions favorable for a specific binding of B cells to the desired antigen. Whereas any means resulting in a stable and specific association of B cells with the desired antigen can be employed, immunization of B cells is preferably performed by co-culturing B cells with the antigen in an immunization medium. In general, the antigens are immobilized on the solid substrate where the B cells are grown. In certain embodiments of the present invention, the desired antigens are presented by a monolayer of cells expressing such antigens. Any cells capable of growth in culture are candidate cells for antigen presentation. Methods and examples of in vitro immunization/activation of non-transformed human B cells are described in U.S. Pat. No. 6,541,225.

Antibodies, CDRs, etc. may also be selected or screened as is known in the art. For instance, CDR libraries may be screened with conditioned cells or membrane fragments thereof to select binding entities against altered antigens. Such libraries and methods are well known in the art. In one embodiment, CDR libraries may be screened against an array of conditioned cells or derived membrane fragments, said array including those described herein.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

The present application also contemplates affinity matured antibodies and fragments thereof that modulate antigens and block ligand activation of an antigen or epitope. The parent antibody may be a human antibody or a humanized antibody. The affinity-matured antibody preferably binds to the target antigen with an affinity superior to that of an intact native antibody (e.g. from about two or about four fold, to about 100 fold or about 1000 fold improved affinity, e.g. as assessed using an antigen sequence ELISA).

The antibodies suitable for use according to the methods herein may further comprise humanized antibodies or human antibodies.

b. Humanized Antibodies

Humanized forms of non-human (e.g., murine) antibodies are chimeric immunoglobulin, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin.

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Human antibodies can also be produced using various techniques known in the art, including phage display libraries (Hoogenboom and Winter, J. Mol. Biol., 227:381 (1991); Marks et al., J. Mol. Biol, 222:581 (1991)). The techniques of Cole et al. and Boemer et al. are also available for the preparation of human monoclonal antibodies (Cole et al., Monoclonal Antibodies and Cancer Therapy, Alan R. Liss, p. 77 (1985) and Boemer et al., J. Immunol., 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg and Huszar, Intern. Rev. Immunol. 13:65-93 (1995). Human antibodies may also be generated by in vitro activated B cells (see e.g. U.S. Pat. Nos. 5,567,610 and 5,229,275).

The antibodies suitable for use in the treatment methods of the invention may further comprise multi-specific including but not limited to bispecific antibodies.

c. Bispecific Antibodies

Bispecific antibodies are monoclonal, preferably human or humanized, antibodies that have binding specificities for at least two different antigens. For example, one of the binding specificities may be for one antigen from a cell that has been treated with a therapeutic agent, the other one is for any other antigen, and preferably for a cell-surface antigen.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism.

According to another approach described in WO 96/27011, the interface between a pair of antibody molecules can be engineered to maximize the percentage of hetero-dimers that are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end products such as homodimers.

Bispecific antibodies can be prepared as full-length antibodies or antibody fragments (e.g. F(ab′)₂ bispecific antibodies). Techniques for generating bispecific antibodies from antibody fragments have been described in the literature. For example, bispecific antibodies can be prepared can be prepared using chemical linkage. Brennan et al., Science 229:81 (1985) describe a procedure wherein intact antibodies are proteolytically cleaved to generate F(ab′)₂ fragments. The bispecific antibodies produced can be used as agents for the selective immobilization of enzymes.

Fab′ fragments may be directly recovered from E. coli and chemically coupled to form bispecific antibodies. Methods to recover Fab′ fragments from E. coli and chemically coupled to form bispecific antibodies are known in the art.

Techniques for making and isolating bispecific antibody fragments directly from recombinant cell culture are also known in art.

Exemplary bispecific antibodies may bind to two different epitopes on a given polypeptide (e.g. cell surface antigen) identified herein. Alternatively, one arm of the antibody, which binds to target polypeptide, may be combined with an arm which binds to a triggering molecule on a leukocyte such as a T-cell receptor molecule (e.g. CD2, CD3, CD28, or B7), or Fc receptors for IgG (FcgR), such as FcgRI (CD64), FcgRII (CD32) and FcgRIII (CD16) so as to focus cellular defense mechanisms to the cell expressing the particular polypeptide. Bispecific antibodies may also be used to localize cytotoxic agents to cells that express a particular antigen identified in accordance with the present invention. These antibodies possess an antigen-binding arm and an arm that binds a cytotoxic agent or a radionuclide chelator, such as EOTUBE, DPTA, DOTA, or TETA. Another bispecific antibody of interest binds the target antigen and further binds tissue factor (TF).

The use of heteroconjugate antibodies is also within the scope of the present invention.

d. Heteroconjugate Antibodies

Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

It may be desirable to modify the antibody of the invention with respect to effector function, so as to enhance, e.g., the effectiveness of the antibody in treating cancer.

e. Effector Function Engineering

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation. “Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-Bearing target cell and subsequently kill the target cell with cytotoxins. The antibodies “arm” the cytotoxic cells and are absolutely required for such killing. The primary cells for mediating ADCC, NK cells, express Fc.gamma.RIII only, whereas monocytes express Fc.gamma.RI, Fc.gamma.RII and Fc.gamma.RIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. (USA) 95:652-656 (1998).

“Fc receptor” or “FcR” describes a receptor that binds to the Fc region of an antibody. The preferred FcR is a native sequence human FcR. Moreover, a preferred FcR is one that binds an IgG antibody (a gamma receptor) and includes receptors of the Fc.gamma.RI, Fc.gamma.RII and Fc.gamma.RIII subclasses, including allelic variants and alternatively spliced forms of these receptors. Fc.gamma.RII receptors include Fc.gamma.RIIA (an “activating receptor”) and Fc.gamma.RIIB (an “inhibiting receptor”), which have similar amino acid sequences that differ primarily in the cytoplasmic domains thereof. Activating receptor Fc.gamma.RIIA contains an immunoreceptor tyrosine-Based activation motif (ITAM) in its cytoplasmic domain. Inhibiting receptor Fc.gamma.RIIB contains an immunoreceptor tyrosine-Based inhibition motif (ITIM) in its cytoplasmic domain. (see review M. in Daron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Human effector cells” are leukocytes that express one or more FcRs and perform effector functions. Preferably, the cells express at least Fc.gamma.RIII and perform ADCC effector function. Examples of human leukocytes that mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) that are bound to their cognate antigen. To assess complement activation, a CDC assay, e.g., as described in Gazzano-Santoro et al., J. Immunol. Methods 202:163 (1996), may be performed.

It may be desirable to modify the antibody of the invention with respect to effector function, e.g., so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989). To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG.sub.1, IgG.sub.2, IgG.sub.3, or IgG.sub.4) that is responsible for increasing the in vivo serum half-life of the IgG molecule.

The invention also includes the use of immunoconjugates.

f. Immunoconjugates

The invention also includes the use of immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of calicheamycins, maytansines, auristatin, duocarmycin, gelosin, saponin, diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and I¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. Conjugates of an antibody and one or more small molecule toxins, such as a calicheamicin, maytansinoids, a trichothene, and CC1065, and the derivatives of these toxins that have toxin activity, are also contemplated herein.

In another embodiment, the antibody may be conjugated to a “receptor” (such streptavidin) for utilization in cell pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

Methods to prepare immunoconjugates are known in art.

The antibodies used in the treatment methods of the present invention may also be formulated as immunoliposomes.

g. Immunoliposomes

A “liposome” is a small vesicle composed of various types of lipids, phospholipids and/or surfactant that is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA 82:3688 (1985); Hwang et al., Proc. Natl Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. A chemotherapeutic agent (such as Doxorubicin) is optionally contained within the liposome.

The antibodies herein may also be used in ADEPT.

h. Antibody-Dependent Enzyme Mediated Prodrug Therapy (ADEPT)

The antibodies herein may also be used in ADEPT by conjugating the antibody to a prodrug-activating enzyme that converts a prodrug (e.g., a peptidyl chemotherapeutic agent,) to an active drug. Methods for conjugating an antibody to a prodrug are known in the art.

The enzyme component of the immunoconjugate useful for ADEPT includes any enzyme capable of acting on a prodrug in such as way so as to convert it into its more active form.

Enzymes that are useful in the method of this invention include, but are not limited to, glycosidase, glucose oxidase, human lysosyme, human glucuronidase, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug 5-fluorouracil; proteases, such as serratia protease, thermolysin, subtilisin, carboxypeptidases (e.g., carboxypeptidase G2 and carboxypeptidase A) and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as beta-galactosidase and neuraminidase useful for converting glycosylated prodrugs into free drugs; beta-lactamase useful for converting drugs derivatized with beta-lactams into free drugs; and penicillin amidases, such as penicillin Vamidase or penicillin G amnidase, useful for converting drugs derivatized at their amine nitrogens with phenoxyacetyl or phenylacetyl groups, respectively, into free drugs. Alternatively, antibodies with enzymatic activity, also known in the art as “abzymes” can be used to convert the prodrugs of the invention into free active drugs. Antibody-abzyme conjugates can be prepared as described herein for delivery of the abzyme to a cell population.

The enzymes can be covalently bound to the antibodies by techniques well known in the art. Alternatively, fusion proteins comprising at least the antigen binding region of the antibody of the invention linked to at least a functionally active portion of an enzyme of the invention can be constructed using recombinant DNA techniques well known in the art.

Antibodies produced by the methods described above are characterized and selected for their ability to bind antigens presented by a cell (e.g. neoplastic cell) that has been exposed to a specific treatment or deleterious stimuli.

The ideal population of antibodies generated with the methods described herein exhibits the following characteristics: (a) lacks substantial immunological reactivity with serum biomolecules; (b) binds to surface antigens that are representative of the type of cells used for immunization; and (c) contains at least one monoclonal or polyclonal antibody reactive with an antigen that is presented by a cell (e.g. neoplastic cell) which has been exposed to a specific treatment or deleterious stimuli.

The lack of substantial immunological reactivity is determined by testing the population of monoclonal antibodies against total serum biomolecules. A population of monoclonal antibodies is deemed to lack substantial immunological reactivity if it yields no detectable binding to total serum biomolecules when used with a dilution about 1:10,000, preferably about 1:1000, more preferably about 1:500. The total serum may be tested at a concentration about 0.001% (v/v), preferably at a concentration about 0.01%, more preferably at 0.1% and even more preferably at 1%. Antigen binding can be detected by immunoassays including, e.g., ELISA and immunoblotting assays. Preferably, the detection is carried out by inumunoblotting total serum biomolecules resolved by electrophoresis on a reducing polyacrylamide gel.

The ability of the population of monoclonal or polyclonal antibodies to recognize surface antigens presented by a cell that has been undergone cellular conditioning, can be tested against viable and intact cells of that particular type, which present surface antigens. For example, antibodies bound to the surface antigens can be detected directly by immunoassays, for example, by reacting labeled antibodies with viable and intact cells immobilized onto a substrate. In an alternative, binding to surface antigens can be assessed by fluorescence-activated cell sorting (FACS), which involves labeling target cells with antibodies coupled to a detectable agent, and then detecting the labeled cells.

The antibodies of the invention can be bound to many different carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

The antibodies of this invention can also be conjugated to a detectable agent or a hapten. The complex is useful to detect the antigens to which the antibody specifically binds in a sample, using standard immunochemical techniques such as immunohistochemistry as described by Harlow and Lane (1988) supra. There are many different labels and methods of labeling known to those of ordinary skill in the art. Examples of the types of labels that can be used in the present invention include radioisotopes, enzymes, colloidal metals, fluorescent compounds, bioluminescent compounds, and chemiluminescent compounds. Those of ordinary skill in the art will know of other suitable labels for binding to the antibody, or will be able to ascertain such, using routine experimentation. Furthermore, the binding of these labels to the antibody of the invention can be done using standard techniques common to those of ordinary skill in the art.

Another technique that may also result in greater sensitivity consists of coupling the antibodies to low molecular weight haptens. These haptens can then be specifically detected by means of a second reaction. For example, it is common to use such haptens as biotin, which reacts avidin, or dinitropherryl, pyridoxal, and fluorescein, which can react with specific anti-hapten antibodies. See Harlow and Lane (1988) supra.

i. Additional Modifications

Covalent modifications of the target antigens, epitopes and antibodies are included within the scope of this invention. One type of covalent modification includes reacting targeted amino acid residues of a target antigen, epitope or antibody with an organic derivatizing agent that is capable of reacting with selected side chains or the N- or C-terminal residues of the target antigen, epitope or antibody. Derivatization with bifunctional agents is useful, for instance, for crosslinking a target antigen, epitope or antibody to a water-insoluble support matrix or surface for use in the method for purifying target antigens, epitopes or antibodies as binding partners, and vice-versa. Commonly used crosslinking agents include, e.g., 1,1-Bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl-)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the .alpha.-amino groups of lysine, arginine, and histidine side chains (T. E. Creighton, Proteins: Structure and Molecular Properties, W. H. Freeman & Co., San Francisco, pp. 79-86 (1983)), acetylation of the N-terminal amine, and amidation of any C-terminal carboxyl group.

Another type of covalent modification of the target antigens, epitopes or antibodies included within the scope of this invention comprises altering the native glycosylation pattern of the antibody or peptide. “Altering the native glycosylation pattern” is intended for purposes herein to mean deleting one or more carbohydrate moieties found in native sequence target antigen, epitope or antibody (either by removing the underlying glycosylation site or by deleting the glycosylation by chemical and/or enzymatic means), and/or adding one or more glycosylation sites that are not present in the native sequence of the target antigen, epitope or antibody. In addition, the phrase includes qualitative changes in the glycosylation of the native proteins, involving a change in the nature and proportions of the various carbohydrate moieties present.

Glycosylation of antibodies and other polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the target antigen, epitope or antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original target antigen, epitope or antibody (for O-linked glycosylation sites). The target antigen, epitope or antibody amino acid sequence may optionally be altered through changes at the DNA level, particularly by mutating the DNA encoding the target antigen, epitope or antibody at preselected bases such that codons are generated that will translate into the desired amino acids.

Another means of increasing the number of carbohydrate moieties on the target antigen, epitope or antibody is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the target antigen, epitope or antibody may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of a target antigen, epitope or antibody comprises linking the antibody or polypeptide to one of a variety of nonproteinaceous polymers, e.g., polyethylene glycol (PEG), polypropylene glycol, or polyoxyalkylenes, in the manner set forth in U.S. Pat. No. 4,640,835; 4,496,689; 4,301,144; 4,670,417; 4,791,192 or 4,179,337. The antibody or polypeptide also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

The target antigen, epitope or antibody of the present invention may also be modified in a way to form chimeric molecules comprising a target antigen, epitope or antibody fused to another, heterologous polypeptide or amino acid sequence.

In one embodiment, such a chimeric molecule comprises a fusion of the target antigen, epitope or antibody with a tag polypeptide that provides an epitope to which an anti-tag antibody can selectively bind. The epitope tag is generally placed at the amino- or carboxyl-terminus of the target antigen, epitope or antibody The presence of such epitope-tagged forms of the target antigen, epitope or antibody can be detected using an antibody against the tag polypeptide. Also, provision of the epitope tag enables the target antigen, epitope or antibody to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., Mol. Cell. Biol., 8:2159-2165 (1988)); the c-myc tag and the 8F9, 3C7, 6E10, G4, B7 and 9E10 antibodies thereto (Evan et al., Molecular and Cellular Biology, 5:3610-3616 (1985)); and the Herpes Simplex virus glycoprotein D (gD) tag and its antibody (Paborsky et al., Protein Engineering, 3(6):547-553 (1990)). Other tag polypeptides include the Flag-peptide (Hopp et al., BioTechnology, 6:1204-1210 (1988)); the KT3 epitope peptide (Martin et al., Science, 255:192-194 (1992)); an .alpha.-tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)).

In an alternative embodiment, the chimeric molecule may comprise a fusion of a target antigen, epitope or antibody with an immunoglobulin or a particular region of an immunoglobulin. For a bivalent form of the chimeric molecule (also referred to as an “immunoadhesion”), such a fusion could be to the Fc region of an IgG molecule. The Ig fusions preferably include the substitution of a soluble (transmembrane domain deleted or inactivated) form of a target antigen, epitope or antibody in place of at least one variable region within an Ig molecule. In a particularly preferred embodiment, the immunoglobulin fusion includes the hinge, CH₂ and CH₃, or the hinge, CH₁, CH₂ and CH₃ regions of an IgG₁ molecule. For the production of immunoglobulin fusions see also U.S. Pat. No. 5,428,130 issued Jun. 27, 1995.

The sequence of the antibody of the invention can be determined by using phage display. Methods for preparing and using phage libraries are well known in the art. Examples of phage libraries include those commercially available from Dyax Corporation and those in US Application Publication No. 20030104355, US Application Publication No. 20030232333, US Application Publication No. 20040180327 and PCT patent applications WO 96/06213, WO 92/01047, and W091/172271.

Typically, antibody phage libraries combine gene fragments from antibody-producing cells with strategically designed synthetic DNA. Antibody-producing cell fragments encoding the light-chain variable region and a portion of the heavy-chain variable region are combined with synthetic DNA encoding human antibody sequences with diversity introduced at strategic sites. These diverse gene products can be displayed on phage and phagemid libraries as Fab or ScFv antibody fragments. The hybrid antibodies or hybrid antibody fragment that are cloned into a display vector can be selected against the appropriate antigen presented by a conditioned cell as described above. Untreated cells can be used to absorb out antibodies that are not specific to conditioned cells.

The efficacy of the antagonist described herein for the methods of the inventions can be tested in vitro using primary cells or cell lines and/or in vivo using animal, including humans.

### V. Cellular Vaccines

The subject conditioned cells are useful for generating cellular vaccines. A variety of methods for generating cellular vaccines known in the art are applicable (see, e.g., US Application Nos. 20050136066, 20050106130, and 20050260208).

In some aspects, the conditioned cells of this invention are used as a source of purified cell membrane preparations that are used as a vaccine to induce or augment the endogenous immune response of a subject to a tumor. The conditioned cells are desirably cancer stem cells or normal fetal tissue stem cells that express a variety of oncofetal and other antigens commonly expressed by tumors and diseased or damaged cells. In certain aspects, the immunogen consists of plasma membrane fragments or vesicles, preferably oriented right side out and administered to a subject having a disease in such a way as to induce an immune response in said subject. In certain aspects, the immunogen shares epitopes with the subject's diseased, cancerous or damages cells; in other aspects epitopes presented by the immunogen are different.

Administration of polyvalent antisera derived from the conditioned cells of this invention, followed by or contemporaneous with administration of a conditioning agent, is a desirable embodiment of this invention.

In some aspects, one type of antigen presenting cell, dendritic cells, are useful in the area of cancer immunotherapy. Dendritic cells are bone marrow-derived cells that can internalize antigen and process the antigen such that it is presented in the context of both the MHC class I complex and the MHC class II complex. In some aspects, a dendritic cell used in the subject invention is able to activate both CD8+ T cells (which are primarily cytotoxic T lymphocytes) and CD4+ T cells (which are primarily helper T cells). It should be understood that any cell capable of presenting a peptide derived from an internalized antigen on both class I and class II MHC is a dendritic cell of the invention (Steinman, Annu. Rev. Immunol. 9: 271-296 (1991)). In this capacity, dendritic cells can be used to present an antigen of interest to T cells. Several approaches have been adopted to directly load tumor antigens onto dendritic cells, including the pulsing of tumor peptides onto mature dendritic cells (Avigan, Blood Reviews 13: 51-64 (1999)). Isolated dendritic cells loaded with tumor antigen ex vivo and administered as a cellular vaccine have been found to induce protective and therapeutic anti-tumor immunity in experimental animals (Tinunerman et al., Annu. Rev. Med. 50:507-529 (1999).

In one aspect, a conditioned cell differentially expressing at least one antigen or a membrane-derived fragment is allowed to contact in vivo or ex vivo an antigen presenting cell, such as a dendritic cell. Upon uptake of the antigen by the dendritic cell, cellular vaccines are expected to be generated by other lymphocytic cells to which the antigen is presented. In addition to dendritic cells, other cells useful for presenting agents include but are not limited to macrophages, B cells, and other cells fused with a conditioned cell of the present invention.

### VI. Diagnostic Methods

Another aspect of the invention involves methods for the diagnosis and classification of cellular dysfunction, damage and disease conditions, and for the prognosis of the outcome of a treatment for the cellular dysfunction, damage or disease condition, and patient response to a particular treatment modality. The present invention involves using the target gene, protein, antigen, epitope or antigen modulator generated as described herein for the diagnosis and classification of cellular dysfunction, damage and disease conditions and for the prognosis of the outcome of a treatment for the condition, and patient response to a particular treatment modality.

In one embodiment, the antibody populations and/or the hybridomas producing such antibodies of the present invention are used for the diagnosis and classification of a cellular dysfunction, damage or disease condition. In other embodiments, the presence of a target gene, protein, antigen or epitope in a cell is detected according to known methods to assess the prognosis, staging or presence of a disease condition.

For example, a differentially expressed gene, protein, antigen, epitope or modulator directed against target antigens can be used as diagnostics or prognostics of a neoplastic condition. The antigen(s) can be an indication of neoplastic condition recurrence, degree of malignancy, and/or prognosis. The antigen can also be used to select a treatment, predict response of a treatment and/or modulate a treatment.

For example, antibodies, including antibody fragments, can be used to qualitatively or quantitatively determine proteins or lipids altered (e.g. dimerization, lipid aggregation, and apoptosis-related changes) by a treatment. The antibody preferably is equipped with a detectable label, such as fluorescent label, and binding can be monitored by light microscopy, flow cytometry, fluorimetry, or other techniques known in the art. These techniques are particularly suitable, if the antigen is a cell surface antigen, e.g., a receptor. Such binding assays are performed by methods known in the art including those described herein.

The diagnostic assays of the present invention take advantage of the finding that different cells respond differently to different treatments or cellular conditioning (e.g. neoplastic cells to chemotherapeutic agents). Accordingly, the diagnostic assays are performed in conjunction with therapeutic treatment or cellular conditioning (e.g. chemotherapy), where the therapeutic treatment or conditioning may precede, occur concurrently with, or follow the administration of the diagnostic agent targeting a conditioned antigen, such as a corresponding antibody. The difference in response of different cells to different therapeutic treatments or cellular conditioning greatly improves the sensitivity of the diagnostic assay, and may allow the detection of antigens which would otherwise not be detectable. This, in turn, allows early detection of cells that might be resistant to the therapeutic treatment, early intervention using the most appropriate treatment, including targeting the antigen identified and/or treatment modulation.

In situ detection of antibody binding to the marker gene products can be performed, for example, by immunofluorescence or immunoelectron microscopy. For this purpose, a histological specimen is removed from the patient, and a labeled antibody is applied to it, preferably by overlaying the antibody on a biological sample. This procedure also allows for determining the distribution of the marker gene product in the tissue examined. It will be apparent for those skilled in the art that a wide variety of histological methods are readily available for in situ detection.

### VII. Methods of Treatment

Another aspect of the invention involves treating a disease condition with an antigen modulator of a target antigen from a subject who has been exposed to a therapeutic treatment or cellular conditioning. In one embodiment, the antigen modulator is effective in ameliorating the disease condition only upon initiation of the therapeutic treatment. In another embodiment the antigen modulator alone is effective in ameliorating the disease condition upon initiation of the therapeutic treatment. In another aspect, the present invention provides a method comprising administering to a subject diagnosed with a disease an amount of a radiotherapeutic agent, and a modulator of a cell surface antigen that is differentially expressed in a diseased cell relative to a corresponding normal cell by said radiotherapeutic agent. In yet another aspect, the present invention also provides a method involving administering to a subject previously exposed to cellular conditioning an effective amount of a modulator of a cell surface antigen that is differentially expressed in a treated cell relative to corresponding normal cells. The cellular conditioning agent is a substance or external force in a biological setting, the addition or removal of which alters, impedes, stimulates, suppresses, enhances, impairs, interferes with, inhibits or prevents the normal function of cells and/or causes injury, damage or destruction of cells. In preferred embodiments, the cellular conditioner is selected from the group consisting of radioactive isotopes (e.g. At⁻²¹¹, I⁻¹³¹, I⁻¹²⁵, Y⁻⁹⁰, Re⁻¹⁸⁶, Re⁻¹⁸⁸, Sm⁻¹⁵³, Bi⁻²¹², P⁻³² and radioactive isotopes of Lu), chemotherapeutic agents, toxins such as small molecule toxins or enzymatically active toxins of bacterial, fungal, plant or animal origin, including fragments and/or variants thereof, free radicals, electric charge, ischemia, oxidant injury, heat shock, cardiac hypertrophy, fever, inflammation, metabolic diseases, infection, cytokines, growth factors, hormones, pathogens, (e.g., bacteria, parasites, intracellular parasites, fungi, viruses, prions, and viroids), cell-cell interactions, soluble factors, and cell and tissue damage of other causes.

The subject treatment methods can employ a variety of antagonists disclosed herein alone, or in combination with any known treatment in the art. In one embodiment, the antibody populations and/or the hybridomas producing such antibodies of the present invention are used for the treatment of a disease condition In one embodiment, the antigen modulator is effective in ameliorating the disease condition only upon initiation of the therapeutic treatment. In another embodiment the antigen modulator alone is effective in ameliorating the disease condition upon initiation of the therapeutic treatment.

In another embodiment, the antigen modulator is effective in making the diseased or damaged cells more susceptible to complement dependent cytotoxicity (CDC), antibody dependent cell mediated cytotoxicity (ADCC) or other host surveillance immunity. In one embodiment, the antigen modulator is effective in making the diseased or damaged cells more susceptible to CDC, ADCC or other host surveillance immunity only upon initiation of the therapeutic treatment. In another embodiment, the antigen modulator alone is effective in making the diseased or damaged cells more susceptible to CDC, ADCC or other host surveillance immunity.

In another embodiment, an antigen modulator of the present invention is used in combination with another modulator, such as another antibody. In yet another embodiment, an antigen modulator of the present invention is used in combination with a therapeutic agent. Examples of therapeutic agents are described above. It is contemplated by the methods herein that the therapeutic agent used in combination with the antigen modulator may or may not be a different therapeutic agent than the cellular conditioner that affected the production of the antigen in the cell. For example, an antigen modulator of this invention that targets a specific antigen in a neoplastic cell can be used in combination with another antigen modulator that targets a different antigen in the diseased or damaged cell. Alternatively, a population of heterogeneous diseased or damaged cells may be treated with a combination of antigen modulators that target antigens in different cells. In another example, an antigen modulator that binds an antigen in a diseased or damaged cell can be used in combination with chemotherapy or radiation therapy or a combination thereof, wherein the chemotherapy and/or radiation therapy may or may not have affected the differential production of the antigen in the cell. The combination treatments can occur in concert or one treatment may precede the other.

In another embodiment, normal, fetal or progenitor cells are conditioned and express an antigen that, when used in conjunction with an antigen modulator, serves to protect the cell, such as by providing trophic or growth stimulation. For example, if cancer cells do not express the same antigen upon exposure to the same conditioning agent, the antigen modulator may protect normal tissue (e.g. bone marrow) while allowing the conditioning agent to damage or kill the cancer cell.

In other embodiments where a diseased or damaged cell does not express the same antigen to at all or to the same extent as a normal cell, the antigen modulator is used to selectively damage nearby normal tissue. This is useful for example to injure normal endothelial cells near a tumor cell, thus inhibiting angiogenesis and harming the tumor vasculature.

In another embodiment, subjects in need of thereof can cycle to different antigen modulator at different time points of their treatment. For instance, a cancer patient can be given an antigen modulator that binds an early antigen produced in response to a chemotherapeutic agent at an earlier time during the chemotherapy treatment. The same patient is then given another antigen modulator that binds a late antigen produced in response to a chemotherapeutic agent at a later time during the chemotherapy treatment. In another example, a cancer patient can be administered an antigen modulator following treatment with another antigen modulator that was not effective for the patient.

Another aspect of the invention involves personalized treatment with the antigen modulator (s) of the invention, in which the antigen(s) differentially expressed as a result of cellular conditioning of a cell from a subject subjected to the cellular conditioning is determined. Antigen modulator(s) that bind the specific antigen(s) produced in the subject are then administered to the subject according to the methods described herein. In one embodiment, the antigen modulators of the present invention are used for the personalized treatment.

Another aspect of the invention involves sensitizing a cell (e.g. neoplastic cell) to an antigen modulator treatment by pre-treating the cell with a cellular conditioner, wherein the antigen modulator is specific for an antigen produced in the cell in response to the cellular conditioner. In one embodiment, sensitizing includes providing a targeted treatment to a cell (e.g. neoplastic cell) by targeted conditioning of the cell with an agent (e.g. radiation) such an antigen is differentially expressed in the cell, and that cell is then treated with an antigen modulator that specifically binds the antigen. For example, neoplastic cells can be pre-treated with a dose of radiation in order to selectively display antigens in response to the radiation treatment. These pretreated cells can then be treated with an antigen modulator as described herein. Examples of therapeutic agents and cellular conditioners are described above.

In one embodiment, the antigen modulators of the present invention are used for the treatment of a disease condition in a subject when that subject has been pretreated with a therapeutic agent that sensitizes the subject to antigen modulator treatment.

In another embodiment, the antigen modulators of the present invention are used for the targeted treatment of a disease condition in a subject when the subject has been subjected to targeted pre-treatment with a cellular conditioning agent results in differential expression of an antigen that is recognized by the antigen modulator. In yet another embodiment, a dose of radiation is delivered to a neoplastic cell and the neoplastic cells are then treated with an antibody that binds to an antigen that is differentially expressed in the neoplastic cell following exposure to the dose of radiation.

Exemplary disease conditions to be treated with the antigen modulators described herein, such as antibodies, include neoplastic conditions, natural and induced immune deficiency states, autoimmune diseases, cardiovascular disease, neuronal disease, and diseases caused by a variety of pathogens such as virus, parasites, prions, viroids, bacteria, fungi and protozoa.

### Treatment of Neoplastic Conditions:

Neoplastic conditions include benign or malignant tumors (e.g., renal, liver, kidney, bladder, breast, gastric, ovarian, colorectal, prostate, pancreatic, lung, vulval, thyroid, hepatic carcinomas; sarcomas; glioblastomas; and various head and neck tumors); leukemias and lymphoid malignancies; other disorders such as neuronal, glial, astrocytal, hypothalamic and other glandular, macrophagal, epithelial, stromal and blastocoelic disorders; and inflammatory, angiogenic, immunologic disorders and disorders caused by pathogens. Particularly preferred targets for treatment with antigen modulators of the present invention are neoplastic conditions.

The invention provides methods to treat several specific neoplastic conditions. In certain embodiments, the neoplastic conditions are selected from the group including but not limited to adrenal gland tumors, AIDS-associated cancers, alveolar soft part sarcoma, astrocytic tumors, bladder cancer (squamous cell carcinoma and transitional cell carcinoma), bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, dhordoma, chromophobe renal cell carcinoma, clear cell carcinoma, colon cancer, colorectal cancer, cutaneous benign fibrous histiocytomas, desmoplastic small round cell tumors, ependymomas, Ewing's tumors, extraskeletal myxoid chondrosarcoma, fibrogenesis imperfecta ossium, fibrous dysplasia of the bone, gallbladder and bile duct cancers, gestational trophoblastic disease, germ cell tumors, head and neck cancers, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), medulloblastoma, melanoma, meningiomas, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, synovial sarcoma, testicular cancer, thymic carcinoma, thymoma, thyroid metastatic cancer, and uterine cancers (carcinoma of the cervix, endometrial carcinoma, and leiomyoma). In certain preferred embodiments, the cancerous cells are selected from the group of solid tumors including but not limited to breast cancer, colon cancer, prostate cancer, lung cancer, sarcoma, renal metastatic cancer, thyroid metastatic cancer, and clear cell carcinoma.

Carcinoma of the thyroid gland is the most common malignancy of the endocrine system. Carcinoma of the thyroid gland includes differentiated tumors (papillary or follicular) and poorly differentiated tumors (medullary or anaplastic). Carcinomas of the vagina include squamous cell carcinoma, adenocarcinoma, melanoma and sarcoma. Testicular cancer is broadly divided into seminoma and nonseminoma types.

Thymomas are epithelial tumors of the thymus, which may or may not be extensively infiltrated by nonneoplastic lymphocytes. The term thymoma is customarily used to describe neoplasms that show no overt atypia of the epithelial component. A thymic epithelial tumor that exhibits clear-cut cytologic atypia and histologic features no longer specific to the thymus is known as a thymic carcinoma (also known as type C thymoma).

In one preferred embodiment, the invention provides a method of treating breast cancer such as a ductal carcinoma in duct tissue in a mammary gland, medullary carcinomas, colloid carcinomas, tubular carcinomas, and inflammatory breast cancer. Existing treatments available for these breast cancers patients are surgery, immunotherapy, radiation therapy, chemotherapy, endocrine therapy, or a combination thereof. The subject antagonists can be administered after the subject has been treated with any of these treatments or a combination thereof. Upon conditioning by these and other treatments, it is expected that a host of conditioned antigens will be selectively overexpressed. In certain embodiments, the subject antagonists can be administered after the subject has been treated with one or more chemotherapeutic regimens such as doxorubicin, cyclophosphamide, methotrexate, paclitaxel, thiotepa, mitoxantrone, vincristine, or combinations thereof. In other embodiments, the subject antagonists can be administered after the subject has been treated with one or more agents for endocrine therapy such as tamoxifen, megestrol acetate, aminoglutethimide, fluoxymesterone, leuprolide, goserelin, and prednisone.

In another embodiment, the present invention provides a treatment for ovarian cancer, including epithelial ovarian tumors such as adenocarcinoma in the ovary and an adenocarcinoma that has migrated from the ovary into the abdominal cavity. The subject antagonists can be administered after the subject has been treated with any of the existing treatments including immunotherapy, radiation therapy, chemotherapy, endocrine therapy or a combination thereof. Upon conditioning by these and other treatments, it is expected that a host of conditioned antigens will be selectively overexpressed. In certain embodiments, the subject antagonists can be administered after the subject has been treated with one or more chemotherapeutic regimens such as cyclophosphamide, etoposide, altretamine, and ifosfamide. In other embodiments, the subject antagonists can be administered after the subject has been treated with one or more hormone therapy agents such as tamoxifen

Additionally, the invention provides a method of treating cervical cancer such as adenocarcinoma in the cervix epithelial including squamous cell carcinoma and adenocarcinomas. The chief treatments available for cervical cancer are surgery (cryosurgery, a hysterectomy, and a radical hysterectomy), immunotherapy, radiation therapy (external beam radiation therapy or brachytherapy) and chemotherapy. In certain embodiments, the subject antagonists can be administered after the subject has been treated with one or more chemotherapeutic regimens such as cisplatin, carboplatin, hydroxyurea, irinotecan, bleomycin, vincrinstine, mitomycin, ifosfamide, fluorouracil, etoposide, methotrexate, or a combination thereof.

The invention also provides a treatment for prostate cancer, such as a prostate cancer selected from the following: an adenocarcinoma or an adenocarinoma that has migrated to the bone. Surgery, immunotherapy, radiation therapy, cryosurgery, hormone therapy, and chemotherapy are some treatments available for prostate cancer patients. Some radiation therapy options are external beam radiation, including three dimensional conformal radiation therapy, intensity modulated radiation therapy, and conformal proton beam radiation therapy. Brachytherapy and cryosurgery are other possible methods used to treat prostate cancer. The subject antagonists can be administered after the subject has been treated with any of these treatments or a combination thereof. In certain embodiments, the subject antagonists can be administered after the subject has been treated with one or more hormone therapy agents including luteinizing hormone-releasing hormone (LHRH) analogs such as leuprolide, goserelin, triptorelin, and histrelin, and LHRH antagonist such as abarelix. In other embodiments, the subject antagonists can be administered after the subject has been subjected to androgen deprivation therapy or androgen suppression therapy including orchiectomy. In other embodiments, the subject antagonists can be administered after the subject has been treated with an antiandrogen agent such as flutamide, bicalutamide, and nilutamide. In other embodiments, the subject antagonists can be administered after the subject has been treated with one or more chemotherapeutic agents such as doxorubicin, estramustine, etoposide, mitoxantrone, vinblastine, paclitaxel, docetaxel, carboplatin, prednisone or a combination thereof.

The present invention further provides methods of treating pancreatic cancer such as epithelioid carcinoma in the pancreatic duct tissue and an adenocarcinoma in a pancreatic duct. The subject antagonists can be administered after the subject has been treated with any of the existing treatments including chemotherapy and radiation, or a combination thereof. The subject antagonists can be administered after the subject has been treated with one or more chemotherapeutic agents such as 5-fluorouracil (5-FU), mitomycin, ifosfamide, doxorubicin, steptozocin, chlorozotocin, or combinations thereof.

The present invention provides additional methods of treating bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers. In certain embodiments, the subject antagonists can be administered after the subject has been treated with one or more immunotherapy agents such as Bacillus Calmete-Guerin (BCG), interferons, and glycoproteins. In other embodiments, the subject antagonists can be administered after the subject has been treated with one or more chemotherapeutic agents such as thitepa, methotrexate, vinblastine, doxorubicin, cyclophosphamide, paclitaxel, carboplatin, cisplatin, ifosfamide, gemcitabine, or combinations thereof.

Moreover, the present invention provides a treatment for lung cancer such as non-small cell lung cancer (NSCLC), which is divided into squamous cell carcinomas, adenocarcinomas, and large cell undifferentiated carcinomas, and small cell lung cancer. Treatment options for lung cancer include surgery, immunotherapy, radiation therapy, chemotherapy, photodynamic therapy, or a combination thereof. Some possible surgical options for treatment of lung cancer are a segmental or wedge resection, a lobectomy, or a pneumonectomy. Radiation therapy may be external beam radiation therapy or brachytherapy. In certain embodiments, the subject antagonists can be administered after the subject has been treated with one or more chemotherapeutic agents such as cisplatin, carboplatin, paclitaxel, docetaxel, gemcitabine, vinorelbine, irinotecan, etoposide, vinblastine, gefitinib, ifosfamide, methotrexate, or a combination thereof.

The subject treatment methods can be particularly effective in inhibiting growth of a neoplastic cell, especially for those neoplastic cells that have been previously exposed to an anti-cancer agent. The subject methods can also be effective in maintaining or increasing cell susceptibility to an anti-cancer therapeutic agent. Not wishing to be bound by a particular theory, the subject treatments are expected to inhibit growth of a neoplastic cell expressing a cell surface antigen presented by a cell from a subject who has been exposed to an anti-cancer therapeutic agent

The invention provides methods to treat several specific natural and induced immune deficiency states. For example, natural and induced immune deficiency states include B cell (antibody) deficiencies, combined T cell and B cell (antibody) deficiencies, T cell deficiencies, defective phagocytes, complement deficiencies and deficiencies due to the administration of immunosuppressants.

### Treatment of Autoimmune Diseases:

The invention provides methods to treat several specific autoimmune diseases. For example, autoimmune diseases include Acute disseminated encephalomyelitis (ADEM), Addison's disease, Antiphospholipid antibody syndrome (APS), Aplastic anemia, Autoimmune hepatitis, Coeliac disease, Crohn's disease, Diabetes mellitus (type 1), Goodpasture's syndrome, Graves' disease, Guillain-Barré syndrome (GBS), Hashimoto's disease, Lupus erythematosus, Multiple sclerosis, Myasthenia gravis, Opsoclonus myoclonus syndrome (OMS), Optic neuritis, Ord's thyroiditis, Pemphigus, Polyarthritis, Primary biliary cirrhosis, Psoriasis, Rheumatoid arthritis, Reiter's syndrome, Takayasu's arteritis, Temporal arteritis (also known as “giant cell arteritis”), Warm autoimmune hemolytic anemia, Wegener's granulomatosis, Alopecia universalis, Chagas' disease, Chronic fatigue syndrome, Dysautonomia, Endometriosis, Hidradenitis suppurativa, Interstitial cystitis, Neuromyotonia, Sarcoidosis, Scleroderma, Ulcerative colitis, Vitiligo, Vulvodynia.

### Treatment of Cardiovascular Diseases:

The invention provides methods to treat several specific cardiovascular diseases. For example, cardiovascular diseases include Acute Coronary Syndromes such as Unstable Angina and Myocardial Infarction, Arrhythmia, Arteriosclerosis, Cardiopulmonary Arrest, Chest Pain such as Stable Angina, Coronary Heart Disease, Congenital Heart Disease, Heart Failure, Hypertension, Stroke, and Valve Disorders, Arterial Aneurysms, Cardiomegaly, Tachycardia/Bradycardia/Arrhythmia, Cardiac arrest, Cardiomyopathy, Heart Valve Regurgitation (leakage), and Heart Valve Stenosis.

### Treatment of Neurological Diseases:

The invention provides methods to treat several specific neurological diseases. For example, neurological diseases include Absence of the Septum Pellucidum, Acid Lipase Disease, Acquired Epileptiform Aphasia, Acute Dissemninated Encephalomyelitis, ADHD, Adie's Pupil, Adie's Syndrome, Adrenoleukodystrophy, Agenesis of the Corpus Callosum, Agnosia, Aicardi Syndrome, AIDS—Neurological Complications, Alexander Disease, Alpers' Disease, Alternating Hemiplegia, Alzheimer's Disease, Amyotrophic Lateral Sclerosis, Anencephaly, Aneurysm, Angelman Syndrome, Angiomatosis, Anoxia, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar/Spinocerebellar Degeneration, Attention Deficit-Hyperactivity Disorder, Autism, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Cephalic Disorders, Ceramidase Deficiency, Cerebellar Degeneration, Cerebellar Hypoplasia, Cerebral Aneurysm, Cerebral Arteriosclerosis, Cerebral Atrophy, Cerebral Beriberi, Cerebral Gigantism, Cerebral Hypoxia, Cerebral Palsy, Cerebro-Oculo-Facio-Skeletal Syndrome, Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, COFS, Colpocephaly, Coma and Persistent Vegetative State, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Deep Brain Stimulation for Parkinson's Disease, Dejerine-Klumpke Palsy, Dementia, Dementia—Multi-Infarct, Dementia—Semantic, Dementia—Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis Lethar gica, Encephaloceles, Encephalopathy, Encephalotrigeminal Angiomatosis, Epilepsy, Erb-Duchenne and Dejerine-Klumpke Palsies, Erb's Palsy, Fabry's Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fisher Syndrome, Floppy Infant Syndrome, Friedreich's Ataxia, Frontotemporal Dementia, Gangliosidoses, Gaucher's Disease, Gerstmann's Syndrome, Gerstmann-Straussler-Scheinker Disease, Giant Cell Arteritis, Giant Cell Inclusion Disease, Globoid Cell Leukodystrophy, Glossopharyngeal Neuralgia, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Huntington's Disease, Hydranencephaly, Hydrocephalus, Hydrocephalus—Normal Pressure, Hydromyelia, Hypercortisolism, Hypersomnia, Hypertonia, Hypotonia—Infantile, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathy, Iniencephaly, Intestinal Lipodystrophy, Intracranial Cysts, Intracranial Hypertension, Isaac's Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Klüver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lissencephaly, Locked-In Syndrome, Lou Gehrig's Disease, Lupus—Neurological Sequelae, Lyme Disease—Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini-Strokes, Mitochondrial Myopathies, Mobius Syndrome, Monomelic Amyotrophy, Motor Neuron Diseases, Moyamoya Disease, Mucolipidoses, Mucopolysaccharidoses, Multifocal Motor Neuropathy, Multi-Infarct Dementia, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy—Congenital, Myopathy—Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications Of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy—Hereditary, Neurosarcoidosis, Neurotoxicity, Nevus Cavernosus, Niemann-Pick Disease, Normal Pressure Hydrocephalus, Occipital Neuralgia, Occult Spinal Dysraphism Sequence, Ohtahara Syndrome, Olivopontocerebellar Atrophy, Opsoclonus Myoclonus, Orthostatic Hypotension, O'Sullivan-McLeod Syndrome, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Postherpetic Neuralgia, Postinfectious Encephalomyelitis, Post-Polio Syndrome, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudotumor Cerebri, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, SUNCT Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Tuberous Sclerosis, Vascular Erectile Tumor, Vasculitis including Temporal Arteritis, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson's Disease, Wolman's Disease, X-Linked Spinal and Bulbar Muscular Atrophy, and Zellweger Syndrome.

The invention provides methods to treat several specific diseases caused by pathogens such as parasites, prions, viroids, virus, bacteria, fungi and protozoa. For example diseases caused by pathogens include Malaria, Amebiasis, Giardiasis, Trichomoniasis, Trypanosomiasis, and/or Leishmaniasis, helminthiasis, tuberculosis, mycobacterium avium complex disease, leprosy, AIDS, hepatitis, foodborne diseases, influenza, prion diseases, SARS, lyme disease, pneumonia, sexually transmitted infections, and west nile virus,

### Treatment of Oxidative Injuries

Oxidative injury can occur in a cell due to reactive oxygen species (ROS), which include (but are not limited to) superoxide, singlet oxygen, peroxynitrite or hydrogen peroxide. Oxidative injury is known to contribute to tissue injury following irradiation and hyperoxia and is thought to be a cause of a variety of disease including but not limited to neurodegenerative diseases such as Lou Gehrig's disease (aka MND or ALS), Parkinson's disease, Alzheimer's disease and Huntington's disease. Oxidative injury is thought to be linked to certain cardiovascular disease, since oxidation of LDL in the endothelium is a precursor to plaque formation; cancer; aging, inflammatory-immune injuries/autoimmune diseases (rheumatoid arthritis, lupus, and diabetes), AIDS, and adult respiratory distress syndrome. ROS react with nucleic acids, lipids, proteins and sugars. The oxidation of lipids, reducing sugars and amino acids leads to the formation of carbonyls and carbonyl adducts such as 4-hydroxy-2-nonenal (HNE). In addition to forming carbonyl groups, ROS are responsible for deamidation, racemization and isomerization of protein residues.

The subject antigen modulators can be administered after the subject has been exposed to oxidative injury. Upon conditioning cells with oxidative injury, a host of conditioned antigens will be selectively overexpressed to which the subject modulators can be generated. In addition, the subject antigen modulator can be administered after the subject has been treated for oxidative injury with agents such as melatonin; heat treatment; YVB-activated pseudocatalase; and antioxidants including vitamin E, vitamin C, beta carotene, betaine, magnesium, selenium, copper, zinc, manganese, ubiquinone (coenzyme Q), quercetin, phenolic compounds (e.g. phytoestrogens, flavonoids, phenolic acids, butylated hydroxytoluene [BHT]), lycopene, carotenoids, flavonoids, terpenes, limonoids and coumarins.

Several treatments such as antiretroviral therapy, photodynamic treatment and radiation therapy increase oxidative injury. It is contemplated in the methods herein that antigen modulators against oxidative injury antigens can be administered after the subject has been exposed to a treatment that increases oxidative injury.

### Treatment of Hypoxic Conditions:

A variety of pathological conditions exist where the affected tissues are hypoxic or exhibit a low oxygen tension including cancer; inflammation, e.g., chronic inflammatory bowel disease, rheumatoid arthritis and ischemia/reperfusion injury; cystic fibrosis; chronic bronchitis; psoriasis; diabetic vasculopathies and epilepsy. Viable hypoxic cells possess certain characteristics that enable them to survive the adverse conditions in which they are placed. The presence of hypoxia in mammalian tissue causes systemic and metabolic responses, such as increased ventilation, cardiac output and hemopoiesis, as well as biochemical changes, including an increase in anaerobic glycolysis and the level of protective stress proteins. Changes occur in the expression of a variety of biochemical pathways, including cytokines and growth factors like erythropoietin (EPO) and vascular endothelial growth factor (VEGF), glycolytic enzymes and cellular redox regulators such as NADPH. Changes occur in the expression of various transcription factors, such as AP-1, NFkB, and HIF-1.

The subject antigen modulators can be administered after the subject has been exposed to hypoxia. In addition, the subject antigen modulator can be administered after the subject has been treated for hypoxia with agents that include, but are not limited to, quinones, based on the indolequinone nucleus, e.g., mitomycin C and EO9; aromatic N-oxides, e.g., tirapazamine; aliphatic N-oxides, e.g., AQ4N; nitroheterocyclics, such as the cytotoxins CB1954 and SN23862; and agents such as NITP, SR4554 and pimonidazole.

### Treatment of Viral Infections:

In one aspect, the invention provides a method of treating viral infections including but not limited to Genital herpes, herpes zoster, and chickenpox. The subject antigen modulators can be administered after the subject has been treated with common anti-viral agents such as Acyclovir, Famciclovir, and Valacyclovir.

Similarly the invention provides a method of treating Influenza viruses. The subject antigen modulators can be administered after the subject has been treated with Amantadine, Oseltamivir, Rimantadine, Zanamivir, Acetaminophen, and NSAIDs such as aspirin and ibuprofen.

In addition, the invention provides a method of treating Cytomegalovirus infections. The subject antigen modulators can be administered after the subject has been treated with Cidofovir, Fomivirsen, Foscamet, Ganciclovir, and Valganciclovir.

Moreover, the invention provides a method of treating herpes simplex virus infections. The subject antigen modulators can be administered after the subject has been treated with foscarnet, trifluridine, penciclovir, docosanol, tetracaine, acyclovir, valacyclovir, famciclovir, and vidarabine.

Furthermore, the invention provides a method of treating Hepatitis B and C. The subject antigen modulators can be administered after the subject has been treated with common anti-hepatitis agents such as Interferon-alpha and Ribavirin.

Similarly the invention provides a method of treating respiratory syncytial virus. The subject antigen modulators can be administered after the subject has been treated with Ribavirin. Upon conditioning cells this or other treatments, it is expected that a host of conditioned antigens will be selectively overexpressed.

The invention methods can be applied to treat Rhinovirus infections. The subject antigen modulators can be administered after the subject has been treated with pleconaril; Echinacea; zinc preparations; vitamin C; Analgesics/Antipyretics such as aspirin, Acetaminophen, Nonsteroidal anti-inflammatory drugs; Antihistamines such as Brompheniramine, Chlorpheniramine, Clemastine, and Diphenhydramine; Cough suppressants such as Benzonatate, Codeine, and Dextromethorphan; Decongestants such as Naphazoline, Oxymetazoline, Phenylephrine, Xylometazoline, Pseudoephedrine and Guaifenesin; Zinc.

The invention methods can also be applied to treat Severe Acute Respiratory Syndrome (SARS). The subject antigen modulators can be administered after the subject has been treated with antiviral drugs, including oseltamivir and ribavirin, and corticosteroids.

Similarly, the subject antigen modulator can be administered after the subject has been treated with one or more anti-HIV agents, including the commonly used cocktails of therapeutics. Such agent includes but is not limited to the following: a Nonnucleoside Reverse Transcriptase Inhibitor (NNRTI) such as Delavirdine, Efavirenz, Nevirapine; Nucleoside Reverse Transcriptase Inhibitors (NRTIs) such as Abacavir, Lamivudine, Zidovudine, Didanosine, Emtricitabine, Tenofovir DF, Stavudine, and Zalcitabine; Protease Inhibitors (PIs) such as Amprenavir, Atazanavir, Fosamprenavir, Indinavir, Lopinavir, Ritonavir, Nelfinavir, Ritonavir, Saquinavir, Invirase, and Tipranavir; and Fusion Inhibitors such as Enfuvirtide. In addition, the subject antigen modulator can be used to treat West Nile Virus.

The methods provided herein comprise generation of an antigen modulator specific for an antigen that is presented by a cell that has been conditioned. The methods provided herein also comprise administering to a subject who has been subjected to a therapeutic treatment a therapeutically effective amount of an antigen modulator specific for an antigen that is presented by a cell that has been exposed to the therapeutic treatment. The methods provided herein also comprise administering to a subject who has been subjected to cellular conditioning a therapeutically effective amount of an antigen modulator specific for an antigen that is presented by cell that has been exposed to the cellular conditioning in combination with another antigen modulator and/or a therapeutic agent known in the art. The methods provided herein also comprise personalized treatment with antigen modulator (s) of the invention, in which the antigen(s) differentially expressed in response to cellular conditioning in a cell from a subject subjected to the cellular conditioning agent is determined. Antigen modulator(s) that bind the specific antigen(s) produced in the neoplastic cell(s) are then administered to the subject according to the methods described herein.

The methods provided herein also comprise cycling subjects in need of thereof with different antigen modulator at different time points of the treatment. The methods provided herein also comprise sensitizing a cell to an antigen modulator treatment by pre-treating the cell with an agent that resulted in the differential expression of the antigen to which to antigen modulator binds.

The antigen modulator generated by the methods described herein will depend, in part, on the disease condition being treated and the specific treatment. For example, an antigen modulator can be generated for cell surface antigens representative of acute myeloid leukemia after treatment with radiation therapy, monoclonal antibody therapy, chemotherapy, gene therapy, immunotherapy, or a combination thereof.

### VII. Administration

The antigen modulators of the present invention are administered to a mammal, preferably a human in accord with known methods, such as intravenous administration as a bolus or by continuous infusion over a period of time, by intramuscular, intraperitoneal, transmucosal, intracerebrospinal, subcutaneous, intra-articular, intrasynovial, intrathecal, oral, topical, or inhalation routes. Intravenous administration of antibodies is preferred. The antigen modulators of the present invention can be administered as described above in combination with a therapeutic treatment, e.g., chemotherapeutic agent. The therapeutic agent can be administered by the same of different routes of administration.

The therapeutic treatment may precede or follow the treatment with the antigen modulator, or may occur simultaneously. The effective amount of a therapeutic agent can be determined by routine testing. Coadministration includes simultaneous administration, or consecutive administration of the two agents in either order.

Administration in combination can include simultaneous administration of two or more agents in the same dosage form, simultaneous administration in separate dosage forms, and separate administration. That is, the antagonist and the therapeutic agent can be formulated together in the same dosage form and administered simultaneously. Alternatively, the antagonist and the therapeutic agent can be simultaneously administered, wherein both the agents are present in separate formulations. In another alternative, the therapeutic agent can be administered just followed by the antagonist or vice versa. In the separate administration protocol, the antagonist and the therapeutic agent may be administered a few minutes apart, or a few hours apart, or a few days apart.

Regarding neoplastic condition treatment, depending on the stage of the neoplastic condition, neoplastic condition treatment involves one or a combination of the following therapies: surgery to remove the neoplastic tissue, radiation therapy, and chemotherapy. The treatment methods of the present invention improve the therapeutic index of therapeutic agents, such as chemotherapy, and thereby allow the reduction of the effective dose to be administered. Accordingly, the therapeutic methods herein are especially useful in the treatment of elderly patients and others who do not tolerate well the toxicity and side effects of chemotherapy and in metastatic disease where radiation therapy has limited usefulness.

Other therapeutic regimens may be combined with the administration of the anti-cancer agents, e.g., antigen modulators and chemotherapeutic agents. For example, the patient to be treated with such anti-cancer agents may also receive radiation therapy and/or may undergo surgery.

For the prevention or treatment of disease, the appropriate dosage of an antagonist, e.g., an antibody herein will depend on the type of disease to be treated, as defined above, the severity and course of the disease, whether the agent is administered for preventive or therapeutic purposes, previous therapy, the patient's clinical history and response to the agent, and the discretion of the attending physician. The agent is suitably administered to the patient at one time or over a series of treatments.

### IX. Formulations

Various formulations of the antigen modulators of this invention may be used for administration. In some embodiments, antigen modulators may be administered neat. In addition to the pharmacologically active agent, the compositions of the present invention may contain suitable pharmaceutically acceptable carriers comprising excipients and auxiliaries that are well known in the art and are relatively inert substances that facilitate administration of a pharmacologically effective substance or which facilitate processing of the active compounds into preparations that can be used pharmaceutically for delivery to the site of action. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers.

Suitable formulations for parenteral administration include aqueous solutions of the active compounds in water-soluble form, for example, water-soluble salts. In addition, suspensions of the active compounds as appropriate for oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances that increase the viscosity of the suspension and include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulation may be used simultaneously to achieve systemic administration of the active ingredient. Excipients as well as formulations for parenteral and nonparenteral drug delivery are set forth in Remington, The Science and Practice of Pharmacy 20th Ed. Mack Publishing (2000).

Suitable formulations for oral administration include hard or soft gelatin capsules, pills, tablets, including coated tablets, elixirs, suspensions, syrups or inhalations and controlled release forms thereof.

Generally, these agents are formulated for administration by injection (e.g., intraperitoneally, intravenously, subcutaneously, intramuscularly, etc.), although other forms of administration (e.g., oral, mucosal, etc) can be also used. Accordingly, antagonists are preferably combined with pharmaceutically acceptable vehicles such as saline, Ringer's solution, dextrose solution, and the like.

The particular dosage regimen, i.e., dose, timing and repetition, will depend on the particular individual and that individual's medical history. Generally regarding antibodies, a dose of at least about 100 ug/kg body weight, more preferably at least about 250 ug/kg body weight, even more preferably at least about 750 ug/kg body weight, even more preferably at least about 3 mg/kg body weight, even more preferably at least about 5 mg/kg body weight, even more preferably at least about 10 mg/kg body weight is administered.

Empirical considerations, such as the half-life, generally will contribute to the determination of the dosage. Regarding antibodies, which are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is based on reducing the number of neoplastic cells, maintaining the reduction of neoplastic cells, reducing the proliferation of neoplastic cells, or delaying the development of metastasis. Alternatively, sustained continuous release formulations of antigen modulator such as antibodies may be appropriate. Various formulations and devices for achieving sustained release are known in the art.

In one embodiment, dosages for antigen modulators may be determined empirically in individuals who have been given one or more administration(s). Individuals are given incremental dosages of an antigen modulator produced as described herein. To assess efficacy antibodies, a marker of the specific disease, disorder or condition can be followed. In embodiments where the individual has cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or an antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Other formulations include suitable delivery forms known in the art including, but not limited to, carriers such as liposomes. Liposomal preparations include, but are not limited to, cytofectins, multilamellar vesicles and unilamellar vesicles.

In some embodiments, more than one antagonist may be present. Such compositions may contain at least one, at least two, at least three, at least four, at least five different antagonist.

### X. Articles of Manufacture

In another embodiment of the invention, an article of manufacture containing the antigen modulator herein, alone or in combination with a therapeutic or conditioning agent, are provided. The article of manufacture comprises a container and a label. Suitable containers include, for example, bottles, vials, syringes, and test tubes. The containers may be formed from a variety of materials such as glass or plastic. The container holds a composition that is effective for treating a disease condition targeted and may have a sterile access port (for example the container may be an intravenous solution bag or a vial having a stopper pierceable by a hypodermic injection needle). The active agent in the composition is an antigen modulator, preferably an antibody. The label on, or associated with, the container indicates that the composition is used for diagnosing or treating the disease condition of choice. The article of manufacture will further comprise, within the same or a separate container, a therapeutic agent and optionally a pharmaceutically acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

### XI. Identification of the Antigen and Molecular Pathways

Another aspect of the invention involves methods identifying antigens involved in antigen modulator treatment as described herein. The present invention also involves methods for exposing pathways associated with antigen modulator treatment of cells. This allows for the identification of new target molecular end point for the treatment of a disease condition (e.g. neoplastic condition).

In one embodiment, representative targets of the antigen modulators produced as described herein and the molecular pathway involve in antibody treatment are identified.

The antigen modulators embodied in this invention provide specific reagents for isolating and cloning the target antigens. As used herein, the term “isolated” means separated from constituents, cellular and otherwise, in which the antigens or fragments thereof, are normally associated with in nature.

The surface antigen recognized by an antigen modulator can be isolated by a number of processes well known to artisans in the field. Representative procedures are immunoprecipitation and immunoaffinity purification of the target antigens from tissue homogenates or cell lysates. Both methods proceed with binding the target antigens to monoclonal antibodies that are immobilized onto a solid-phase matrix (e.g. protein A and protein G sepharose beads), followed by separating the bound antigens with the unbound proteins, and finally eluting the antigens from the antibody-coupled solid-phase matrix. Subsequent analysis of the eluted antigens may involve electrophoresis for determining the molecular weight, and protein sequencing for delineating the amino acid sequences of the target antigen. Based on the deduced amino acid sequences, the cDNA encoding the antigen can then be obtained by recombinant cloning methods including PCR, library screening, homology searches in existing nucleic acid databases, or any combination thereof. Commonly employed databases include but are not limited to GenBank, EMBL, DDBJ, PDB, SWISS-PROT, EST, STS, GSS, and HTGS.

A preferred method of cloning the target surface antigen is by “panning” the antibodies for cells expressing the cell surface antigen of interest. The “panning” procedure is conducted by obtaining the cDNAs of cells that express the antigen of interest, over-expressing the cDNAs in a second cell type, and screening cells of the second cell type for a specific binding to the monoclonal antibody.

cDNAs can be obtained by reverse transcribing the mRNAs from a particular cell type according to standard methods in the art. Specifically, mRNA can be isolated using various lytic enzymes or chemical solutions according to the procedures set forth in Sambrook et al. (“Molecular Cloning: A Laboratory Manual”, Second Edition, 1989), or extracted by nucleic-acid-binding resins following the accompanying instructions provided by manufacturers. The synthesized cDNAs are then introduced into an expression vector to produce the antigens in cells of a second type. It is implied that an expression vector must be replicable in the host cells either as episomes or as an integral part of the chromosomal DNA. Suitable expression vectors include plasmids, viral vectors, including adenoviruses, adeno-associated viruses, retroviruses, cosmids, etc.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (where the vector is an infectious agent, such as vaccinia virus, which is discussed below). The choice of introducing vectors or polynucleotides will often depend on features of the host cell.

Any host cells capable of over-expressing heterologous DNAs can be used for the purpose of isolating the genes encoding the target surface antigens. Non-limiting examples of mammalian host cells include but not limited to COS, HeLa, and CHO cells. Preferably, the host cells express the cDNAs at a level of about 5 fold higher, more preferably 10 fold higher, even more preferably 20 folds higher than that of the corresponding endogenous antigens, if present, in the host cells. Screening the host cells for a specific binding to the selected monoclonal antibodies is effected by an immunoassay or preferably, FACS.

The antigen modulators embodied in this invention provide specific reagents for determining the molecular pathways involved in the treatment of damage, disorder or disease conditions according to the methods described herein. This allows for the identification of new target molecular end point for the treatment of a disease condition (e.g. neoplastic condition).

In one embodiment, molecular pathways involved in antagonist treatments described herein are determined by gene expression profiling. The most commonly used methods known in the art for the quantification of mRNA expression in a sample include northern blotting and in situ hybridization; RNAse protection assays; and reverse transcription polymerase chain reaction (RT-PCR). Alternatively, antibodies may be employed that can recognize specific duplexes, including DNA duplexes, RNA duplexes, and DNA-RNA hybrid duplexes or DNA-protein duplexes. These techniques or any other technique know in the art can be used to compare mRNA levels in different sample populations, in normal and target tissues (e.g. neoplastic tissue), with or without antagonist treatment, to characterize patterns of gene expression. Methods for DNA and RNA extraction are known in the art.

Differential gene expression can also be identified, or confirmed using microarray techniques. Methods to performed microarrays techniques are well known in the art. In this method, nucleotide sequences of interest are plated, or arrayed, on a microchip substrate. The arrayed sequences are then hybridized with specific DNA probes from cells or tissues of interest. The genes targeted by the present invention can be identified by constructing normalized and subtracted cDNA libraries from mRNA extracted from target cells or tissues (e.g. neoplastic cells), and the normal cells or tissue; purifying the DNA from the cDNA; treating both target and control samples under otherwise identical conditions with an antagonist; microarraying the purified DNA for expression analysis; and probing microarrays to identify the genes from the clones that are selectively upregulated by antagonist treatment in the target cells or tissues, relative to corresponding control cells or tissues (e.g. untreated target cells, treated and untreated normal cells).

Differential gene expression can also be identified, or confirmed using proteomic analysis. Methods to perform proteonomic techniques are known in the art. For instance, differential expression of proteins can be measured using two-dimensional gel electrophoresis. The identity of differentially expressed proteins can then be identified by well-known methods such as sequencing through Edman degradation and mass spectrometry, commonly peptide mass, or De novo repeat detection sequencing. Examples include MALDI-TOF, Q-TOF MS. This results can also be identified, or confirmed using any conventional techniques of immunology, molecular biology, and cell biology such as western blots, ELISA (Enzyme-linked immunosorbent assay), RIAs (Radioimmunoassay), enzyme immunoassays like ELISA, LIA (luminescent immunoassay), and FIA (fluorescent immunoassay).

The gene targets identified in accordance with the present invention and the treatment methods herein can be further validated in cell-based assays. The role of genes and gene products identified herein can be tested by using primary cells or cell lines. Such cells include, for example, breast, colon and lung cancer cells and cell lines listed herein. For example, cells of a cell type known to be involved in a particular tumor are transfected with the cDNAs encoding the proteins identified herein, and the ability of these cDNAs to reduce or inhibit excessive growth or to cause cell death is analyzed. Other cell-based assays are also suitable for validating gene/gene products targets identified in accordance with the present invention and treatment methods. Such cell-based assays include, but are not limited to, cell growth assays (e.g., determination of cell numbers over a period of time, assays based on cell number surrogates such as 3-(4,5-Dimethylthiazol,2-yl)-2,5-dipehenyltetrazolium bromide (MTT), Alamar blue, etc.), cell death assays (e.g., caspase activity assays, intracellular enzyme lactate dehyogenase (LDH) release assays, DNA laddering, etc.), metabolic assays (e.g., ATP assays), cell adhesion assays, colony formation assays (e.g., soft agar), immune activation assays, CDC assays, ADCC assays, and motility assays.

In addition, antigen modulators that bind the gene targets identified in accordance with present invention can be generated for treatment of the condition. Antigen modulators include, but are not limited to, antibodies or fragment thereof, peptides, non-peptide small organic molecules, antisense molecules, and oligonucleotide decoys.

### XII. Business Method

Another aspect of the invention involves a business method in which a therapeutic agent, such a chemotherapeutic agent or radiation, is administered to a subject in order to condition cells involved in a disease condition (e.g. neoplastic cells) to an antigen modulator treatment. This allows for the enhancement of the efficacy of the treatment and reduces the dose of the agent, therefore, reducing toxicity. For example, the chemotherapeutic agent may be toxic at the amount necessary for treatment of a neoplastic condition by the therapeutic agent alone. Therefore, by increasing the efficacy of the chemotherapeutic agent with the antigen modulator, a lower non-toxic amount of the chemotherapeutic agent can be used. In one embodiment, the antigen modulators of the present invention are used in the business method.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Examples

### Example 1

### Preparation of Cells as Immunogen

Whole cells isolated from tissue or from cell culture were used as an immunogen for producing monoclonal antibodies that are specific for surface antigens present on a particular cell type. Such methods suitable for the practice of this invention are well known in the art, and for example, are described in U.S. patent application Ser. No. 10/672,878. Generally, to produce monoclonal antibodies directed to cell-surface antigens of a specific cell type, it is desirable to immunize mice with viable and intact cells of that type, preferably with those cells whose surfaces that are free of serum. Cell lines that are suitable for the generation of monoclonal antibodies against the conditioned antigens of this invention include without limitation: SKBR3 (ATCC #HTB-30), SKMES-1 (ATCC #HTB-58), ES-2 (ATCC #CRL-1978), BT474 (ATCC #HTB-20), HPAF-II (ATCC #CRL-1997), Colo205 (ATCC #CCL-222), SW480 (ATCC #CCL-228), A498 (ATCC #HTB-44), PC-3 (ATCC #CRL-1435), Hs746T (ATCC #HTB-135), AsPC-1 (ATCC #CRL-1682), Hs700T (ATCC #HTB-147), and HT-29 (ATCC #HTB-38).

The cells were grown in the appropriate nutrient media supplemented with growth factors, but free of serum. Immunization with cells that have been propagated in a serum-supplemented medium can have extreme disadvantages. Serum contains a complex mixture of small and large biomolecules with undefined activities. These biomolecules can adhere to the surfaces of cells and thereby leading to the generation of antibodies cross-reacting with molecules not representative of the specific cell type. Additionally, binding of serum biomolecules to the cell surface may lead to the masking of desired cell surface antigen targets. A number of serum-free media preparations are commercially known and publicly available, such as for example, F12/DME (1:1) nutrient media with the following supplements: insulin (10 μg/ml final concentration), epidermal growth factor (EGF) (5 ng/ml final concentration), selenious acid (2.5×10⁻⁸ M final concentration), and porcine pituitary extract (PPE) (5 μl/ml final concentration).

For cells conditioned by radiation, the cells were grown in the appropriate nutrient media to the desired cell density. Prior to radiation exposure, fresh nutrient media was added to the cells. The cells were then exposed to the desired amount of radiation. Specifically, the cells were exposed to 20-200 rads of radiation. Cells were either immediately harvested for immunization or were allowed to recover for 48 hours and then harvested for immunization.

For cells conditioned by exposure to chemotherapeutics or other anti-cancer drugs, cells were grown in the appropriate nutrient media to until 75%-90% confluent. The desired amount of chemotherapeutic or anti-cancer drug was added to the cells. For example, 0.001 μg/ml to 0.1 μg/ml Velcade (final concentration) was added to cells for 48 hours. The concentration of chemotherapeutic or anti-cancer drug and the duration of treatment varied with different cell lines or cell types. After treatment for the desired duration (usually 48 hours), the cells were harvested for immunization.

To harvest the cells, the cell monolayers were rinsed once with calcium- and magnesium-free Hanks saline solution, incubated in 10 mM EDTA in Hanks saline solution at 37 C for 15 minutes. The cells were detached from the culture surface by gentle pipetting. The cell suspension was pelleted by centrifugation at 1000×g for 5 minutes. The supernatant was removed and cells were resuspended in the appropriate medium.

### Example 2

### Generation of Monoclonal Antibodies

After the cells were conditioned and harvested as described in Example 1 above, a non-denaturing adjuvant (Ribi, R730, Corixa, Hamilton Mont.) was rehydrated to 2 ml in phosphate buffered saline. 500 μl of this rehydrated adjuvant was then gently mixed with some of the cell pellet to be used for immunization. Approximately 10⁶ cells per mouse were injected into Balb/c mice via footpad, approximately once or twice a week. The precise immunization schedule is as follows: Days 0, 7, 14, 21 immunization plus RIBI.

At Day 28, blood was drawn from the tail of each immunized mouse to test for sero-conversion against the immunizing cell of choice using flow cytometry (FACSCalibur, BD). When the titer reached at least 1:16K-1:32K, the mice were sacrificed using CO₂, followed by cervical dislocation. Lymph nodes were harvested for hybridoma preparation.

Lymphocytes from immunized mice were fused with mouse myeloma line X63-Ag8.653 using 35% polyethylene glycol. Four to seven days post fusion, the single-cell colonies were transferred from semi-solid medium to 96-well plates for continued growth. Four days after transfer, supernatant was analyzed for the presence of monoclonal antibodies specific against the immunizing cell of choice using flow cytometry. The immunizing cells of choice were prepared in the same manner as described in Example 1. Conditioned medium from each hybridoma was incubated for 30 minutes with an aliquot of the immunizing cell of choice. After incubation, the samples were washed, resuspended in 0.1 ml diluent containing 1 μg/ml of FITC-conjugated goat anti-mouse IgG Fc gamma specific secondary antibody for 30 minutes at 4° C. The samples were then washed, resuspended in 0.2 ml FACS diluent and analyzed using flow cytometry. Positive clones were expanded and reanalyzed using the above described flow cytometry procedure using 1 μg/ml FITC-conjugated F(ab′)2 fragment goat anti-mouse IgG (H+L) before continuing with further analysis. A hybridoma making a monoclonal antibody that binds a desired antigen was selected.

### Example 3

### Purification of Antibodies

Those hybridomas that produce antibodies that continue to be reactive against the immunizing cells of choice are scaled for purification of the antibody. Positive hybridomas were scaled into three T75 flasks. Once the confluent, the cells and supernatant were collected into 50 ml conical tubes and centrifuged. The supernatant was aspirated and the cell pellet was washed with F12/DME (50:50) media and re-centrifuged. The cell pellet was resuspended in 50 ml growth medium, F12/DME (50:50) containing 1% ultra low IgG FBS (GIBCO, Invitrogen Corp.) 10 μg/ml rh insulin, and 10 μg/ml transferrin. Cells were then inoculated into the pouch of a CL1000 (IBS Integra BioScience), and the outer chamber was filled with 500 ml of the same medium. The outer chamber is changed on day 7 and day 14 with fresh growth medium.

The antibody-containing medium was harvested from the cell pouch on day 21. 25 ml of harvested material is mixed 1:1 with load buffer (3M NaCl, 1.5M glycine pH 9.0). The material was flowed over a prepared 5 ml column of protein A resin (Amersham)). The column was then washed with 20 column volumes of phosphate-buffered saline (PBS). The antibodies were eluted with 0.1M glycine, pH 2.8 and neutralized in 20 μl of 3M Tris, pH 9.0. The antibodies were then dialyzed and the protein concentration was determined by A280 readings on a Beckman DU530 spectrophotometer.

### Example 4

### Generation of Monoclonal Antibodies to Altered Antigens

Using the methods described in Example 2, tumor cell lines were pre-treated with sub-lethal doses of Velcade, gemcitabine, and irradiation. Antibodies purified from Example 3 were screened for differential binding to conditioned and control cells. After treatment, the still-living cells were used as immunogens according to the methods described in Example 1. Resulting MAbs were screened first for binding to untreated tumor cells and then those that did not bind to untreated tumor cells were screened for binding to treated cells. The positive clones were grown up and antibody produced and purified for further study.

Multiple MAbs were obtained that bound to the surface of treated, but not untreated cells. In data not shown, more than 75 monoclonal antibodies have been obtained that specifically bound to antigens on the surface of prostate cancer cells that were altered by Velcade. Additionally more than sixty MAbs have been obtained that were specifically altered on one or more of a number of cell lines by irradiation.

Of the 75 MAbs up-regulated by Velcade, fifteen were not found on any of the normal tissues screened. An additional thirteen were found only at low levels on non-essential tissues.

### Example 5

### Up-Regulation of Cell Surface Antigens by Therapeutic Agents

Over one hundred monoclonal antibodies known to be preferentially expressed on cancer cells were evaluated for their alteration on several cell lines [lung (CA130, 9926), colon (CRCA1004, RECA0515, prostate (PRCA1004, PRCA629A), and mantle cell lymphoma (MCL)] by three classes of therapeutic agents including Velcade (a proteosome inhibitor), gemcitabine (a microtubule inhibitor), cisplatin, and irradiation. In all cases a set of antigens was identified whose level was increased by treatment with the therapeutic agent, and another set identified whose level was decreased by the treatment, indicating that these treatments selectively alter the cell surface of surviving tumor cells.

As described in Example 1, cells from several cancer cell lines [lung (CA130, 9926), colon (CRCA1004, RECA0515, prostate (PRCA1004, PRCA629A), and mantle cell lymphoma (MCL)] were grown at 1 μg/ml in the appropriate nutrient media supplemented with growth factors, but free of serum. Cells were grown with and without the presence of members of three classes of therapeutic agents including Velcade, gemcitabine, cisplatin, and irradiation.

Cell surface antigens differentially expressed by conditioning of the cells with these therapeutic agents as described in Example 2 were detected by contacting the cells with more than one hundred test antibodies generated from immunizing mice using the methods of this invention and that were known to bind to cell surface antigens that are preferentially expressed on cancer cells, and then determining the ability of the test antibodies to bind to the conditioned cells and control cells. These antibodies were screened for differential binding to conditioned and control cells using FACs analysis. Methods for FACs analysis is well known in the art. In these experiments, the antibody was incubated for 30 minutes with a suitable amount of the conditioned cells and control cells (about 10⁶ cells). After incubation, the cells were washed and may be optionally fixed with paraformaldehyde. The cells were then resuspended in 0.1 ml diluent containing 1 μg/ml of FITC-conjugated goat anti-mouse IgG Fc gamma specific secondary antibody for 30 minutes at 4 degrees Celsius. The samples were then washed, resuspended in 0.2 ml FACs diluent and analyzed using flow cytometry. Binding of the test antibodies was determined as described above.

In all cases we were able to identify a set of antigens whose level was increased by treatment with the therapeutic agent, suggesting that these treatments do selectively alter the cell surface of surviving tumor cells.

In data not shown some antigens were up-regulated by more than one treatment, and one was up-regulated by all four treatments, but the overall set of up-regulated antigens was treatment and tumor type specific. Cell lines derived from lung cancer and from prostate cancer treated with cis-platin shared some up-regulated antigens and had others that were cell type specific. Similarly, the majority of antigens were up-regulated by irradiation on more than one of the tumor lines tested while a very few were unique to one cell line. Looking at the two different prostate cancer cell lines treated with Velcade, it was seen that some antigens were up-, or down-, regulated on both lines, others changed only on one or the other. In one embodiment, five of the MAbs that are up-regulated have been shown to have biological activity in vitro (data not shown) and are directed to novel targets.

### Example 6

### Cells Conditioned by Radiation

Antibodies purified from Example 3 were screened for differential binding to conditioned and control cells. Other monoclonal antibodies against cell surface targets may also be used to screen for differential binding to conditioned and control cells. The conditioned cells used to immunize and produce these antibodies may be used in the screening. For example, cells from limited passage prostate tumor cell line (PRCA1004) and mantle cell lymphoma cell line (MCL) were grown in the appropriate nutrient media Cells were conditioned by exposure with 20, 50, and 200 rads of radiation. Cells were prepared for FACs analysis using methods described in Example 5. A panel of 125 monoclonal antibodies directed against cell surface antigens was tested on radiation conditioned and control cells. Three monoclonal antibodies demonstrated binding to cells conditioned with all three doses of radiation, with minimal expression on control cells.

### Example 7

### Western Blot Analysis of Antigen Expression in Cancer Cell Lines

Control and conditioned cells prepared under methods described in Example 1 were grown in the appropriate nutrient media to confluency on 175 cm² culture dishes. The confluent monolayer was washed three times with Hank's Balanced Salt Solution (HBSS+ containing no sodium bicarbonate or phenol red; buffered with 10 mM HEPES, pH 7.4; Sigma Chemicals) and biotinylated with 200 μg of sulfo-NHS-LC-biotin (Pierce Endogen) for 30 minutes at room temperature. The cells were then washed with HBSS+ containing 0.1M Tris, pH 7.4 (Sigma Chemicals) and incubated in HBSS+ containing 0.1M Tris, pH 7.4 for 15 minutes at room temperature. The cells were finally washed three times with HBSS+ and lysed by incubation for 5 minutes, on ice, in lysis buffer (HBSS+ with 2% Triton X-100, 2 mM PMSF, 0.1% sodium azide, and 1 tablet per 5 ml lysis buffer of EDTA free complete mini-protease cocktail (Roche Molecular Biochemicals)).

Cells were scraped in lysis buffer and lysates collected. Lysates were centrifuged at 14,000-× g for one hour at 4° C. The clarified lysate was then pre-cleared for 2 hours at 4° C. with 5 μl of human IgG conjugated (1 mg/ml) CNBr 4 MB Sepharose beads (Amersham Pharmacia). Human IgG beads were centrifuged and removed, and then the pre-cleared lysate was then incubated with target monoclonal antibody conjugated to CNBr 4 MB sepharose beads (conjugated at 1 mg/ml) for 2 hours at 4° C. The target antibody beads were centrifuged and removed after the 2 hour incubation. Both the human IgG and the target antibody beads were individually washed three times with 1 ml of lysis buffer and then washed three times with 1 ml HBSS+. The washed beads were eluted by the addition of 30 μl of SDS-PAGE sample buffer and boiling at 99° C. for 5 minutes.

The samples were then resolved on a 4-20% Novex gradient gel (Invitrogen), and transferred onto 0.2 cm nitrocellulose membrane (Invitrogen) and visualized by western blotting with 2 ng/blot strepavidin-HRP (Pierce). Results using this protocol and the test antibodies confirmed the presence or absence of cell surface antigens on control and conditioned cells.

Target antibodies were also analyzed using western blotting with cell lysates that have not been biotinylated. Control and conditioned cells prepared under methods described in Example 1 were grown in the appropriate nutrient media to confluency on 175 cm2 culture dishes. The confluent monolayer was washed three times with HBSS+ containing no sodium bicarbonate or phenol red; buffered with 10 mM HEPES, pH 7.4 (Sigma Chemicals). The cells were then lysed by incubation for 5 minutes, on ice, in lysis buffer (HBSS+ with 2% Triton X-100, 2 mM PMSF, 0.1% sodium azide, and 1 tablet per 5 ml lysis buffer of EDTA free complete mini-protease cocktail (Roche Molecular Biochemicals)). Cells were scraped in lysis buffer and lysates were collected and treated as described above with control (human IgG conjugated CNBr 4 MB sepharose beads) and target monoclonal antibody conjugated to CNBr 4 MB sepharose beads. The target antibody beads were centrifuged and removed after the 2 hour incubation. Both the human IgG and the target antibody beads were individually washed three times with 1 ml of lysis buffer and then washed three times with 1 ml HBSS+. The washed beads were eluted by the addition of 30 μl of SDS-PAGE sample buffer and boiling at 99° C. for 5 minutes. The samples were then resolved on a 4-20% Novex gradient gel (Invitrogen), and transferred onto 0.2 μm nitrocellulose membrane (Invitrogen) and visualized by western blotting.

For western blotting with antibody the nitrocellulose was blocked for 1 hour in blocking buffer. The nitrocellulose was then incubated in a heat sealed plastic pouch containing 1 ml of 5 μg/ml antibody diluted in blocking buffer. The nitrocellulose was washed 3 times with TBST before incubation with 10 ml of 1 μg/ml HRP conjugated donkey anti-mouse IgG (heavy and light chain specific, cross adsorbed against bovine, chicken, goat, guinea pig, Syrian hamsters, horse, human, rabbit, sheep serum proteins (Jackson Immunoresearch Cat. #709-035-149) for 1 hour at room temperature. The nitrocellulose was finally washed three times with TBST and visualized by ECL+ (Amersham). Results using this protocol and the test antibodies confirmed the presence or absence of antigens on control and conditioned cells.

### Example 8

### Immunohistochemistry Methods

Antibodies such as the conditioned cell antibodies of this invention were screened on frozen normal and tumor tissue samples from surgical biopsies and/or autopsy specimen. The frozen tissue samples were embedded in OCT compound and quick-frozen in isopentane with dry ice. Cryosections were cut with a Leica 3050 CM mictrotome at thickness of 8-10 μm and thaw-mounted on SuperFrost Plus slides (VWR #48311-703) and were allowed to air-dry for 2 hours at room temperature. The sections were fixed with 75% acetone/25% ethanol for 10 minutes at room temperature and allowed to air-dry 1-2 hours at room temperature. The fixed sections were stored at −80° C. until use.

For immunohistochemistry, the tissue sections were retrieved, allowed to slowly equilibrate to room temperature from −80° C., washed in Tris buffered 0.05% Tween (TB-T) three times for 5 minutes each time, and blocked in blocking buffer (TB-T, 5% normal goat serum and 100 μg/ml avidin) for 20 minutes at room temperature. The slides were then incubated with the target antibody and control monoclonal antibodies diluted in blocking buffer (1-5 μg/ml) for 60-90 minutes at room temperature or overnight at 4° C. The sections were then washed three times with the blocking buffer and then blocked with a hydrogen peroxidase (1-3%) biotin (d-biotin, 30 μg/ml) block in TB-T was done for 20 minutes at room temperature. A third wash with TB-T was then performed three times as described above. The sections were incubated in blocking buffer with a secondary biotinylated goat anti-mouse IgG+IgM (H+L) antibody at room temperature for 30 minutes. Another wash was performed with TB-T three times at 5 minutes each. Avidin biotin peroxidase complex formation was then accomplished by adding the pre-made ABC solution (Vexation ABC Elite Kit) according to manufacturer's directions and the slides were incubated for 30 minutes at room temperature. Slides were then washed 2 times with TB-T and then once with deionized water for 5 minutes each. A DAB substrate (10 ml 10×DAB solution, 90 ml Tris buffer, pH 7.6, and 100 μl 3% H2O2) was added to the slides for 30 minutes at room temperature to allow for the formation of colored precipitate and visualization of the bound primary antibody. The slides were then counterstained with hematoxylin.

The binding of antibody to a variety of normal human and human tumor tissues was assessed. The results were scored as “±” for equivocal staining, “1+” for weak positive staining, “2+” for moderate positive staining, “3+” for strong positive staining.

### Example 9

### Immunocytochemistry Results

Monoclonal antibodies of this invention were used to test reactivity with various cell lines from different types of tissues. Identical immunohistochemistry protocols as described above were used for staining the CellArray. The results were scored as ‘+’ for weak positive staining, ‘++’ for moderate positive staining, ‘+++’ for strong positive staining and ‘−’ for negative staining.

Immunohistochemistry results were obtained using CellArray™ technology, as described in WO 01/43869. Cells from different established cell lines were removed from the growth surface without using proteases, packed and embedded in OCT compound. The cells were frozen and sectioned, then stained using a standard IHC protocol.

### Example 10

### Internalization of Antibody and Toxin-Conjugated Anti-Mouse IgG

Mab-ZAP (Advanced Targeting Systems, San Diego, Calif.) is an anti-mouse IgG conjugated to saporin, a toxin that inhibits protein synthesis. This toxin is impermeable to the cell membrane. If a monoclonal antibody is bound to a cell-surface antigen that is internalizable, the toxin-conjugate can bind to the bound monoclonal and, thereby, be internalized and eventually kill the cell. Being dependent upon internalization for demonstration of toxic activity, the Mab-ZAP can serve to evaluate whether or not a given surface antigen will serve as a suitable target for any toxin that is dependent upon internalization to express cell toxic effects. As such, the Mab-ZAP serves as a model for such internalization-dependent toxins such as maytansinoids and chalicheamicins.

For testing the internalization of antigen modulating antibody and saporin conjugated anti-mouse IgG by tumor cells and effect of killing the tumor cells after internalization of saporin, human cell lines were removed from stock flasks with 10 mM EDTA and centrifuged. Cells were resuspended at 50,000/ml in appropriate medium and 100 μl plated per well in 96 well plates. Target antibody was added immediately to appropriate wells as a 10× concentrate, to make a final concentration of 10 ug/ml. After 15 minutes at room temperature Mab-ZAP (Cat. #IT-04, Advanced Targeting Systems, San Diego Calif.) was added to appropriate wells as 10× concentrate, to make final concentrations from 0.001 nM to 10 nM. After 4 days growth, MTT was added (stock 5 mg/ml PBS, 1:10 dilution in well) for 4 hrs at 37° C. The medium was then removed from all wells and 100 μl/well DMSO was added. The plates were gently swirled to solubilize the blue MTT precipitate and the plates were read at O.D. 540 nm.

There was a decrease in MTT staining in cells in the presence of the target antibody as compared to staining in the absence of the target antibody. This indicates that the growth of the cells was inhibited in the presence of the target antibody and Mab-ZAP and these results are indicate that the target antibody and toxin-conjugated anti-mouse IgG were internalized in the cells.

### Example 11

### In Vitro Treatment with Antibodies

The ability of the antibodies to reduce cell number in vitro when grown as a monolayer can be assessed using cell monolayers grown in the presence or absence of varying amounts of test or control purified antibody and the change in cell number assessed using MTT. MTT is a dye that measures the activity of mitochondrial enzymes and correlates with relative viable cell number. Other methods for assessing cell number can also be used. The ability of the antibodies to reduce cell number in vitro can be measured in at least two ways: the effect of the antibody alone on cell number and the synergistic effect of the antibody with the conditioning therapeutic on cell number.

The cells of interest are prepared using methods described in Example 1 with the conditioning therapeutic of choice and plated in 96-well plates. For example, the synergistic effect of the antibody with the conditioning therapeutic on cell number was assessed using PRCA 1004 and PRCA 629a cells that have been conditioned with 0.001 μg/ml Velcade for 48 hours. The conditioned cells were plated at the desired densities in triplicate wells of a 96 well dish. Immediately after plating, the test antibody was added. The cells were incubated at 37 degrees Celsius in a humidified incubator at 5% CO₂/air for 5 days. At the end of the assay, MTT was dissolved in PBS (5 mg/ml) and added directly to wells at 1:10 dilution. Plates were placed back in the incubator for 4 hours. After the incubation, medium was removed and 100 μl DMSO was added to solubilize the MTT precipitate. Plates were read at 540 nm on a platereader. Alternatively, the effect of the antibody alone on cell number can be assessed using a cell line that expresses the cell surface antigen recognized by the test antibody of choice. Using these cells and the MTT methods described above, any inhibitory or growth promoting effects of the antibody alone can be assessed.

### Example 12

### In Vitro Treatment with Antibodies Causes Metabolic Changes

The ability of the antibodies to induce metabolic changes in cells grown in vitro can be assessed using varying amounts of test or control purified antibodies and the change in metabolic activity assessed using an adenosine triphosphate (ATP) detection assay system. Many ATP detection assays are well known in the art and are suitable for measuring metabolic changes. In this case, the ATPlite 1 step luminescence ATP detection assay system (Perkin Elmer) was used. The ATPlite 1 step assay system is based on the production of light caused by the reaction of ATP produced by the cells with added luciferase and D-luciferin. Luciferase catalyzes the ATP/D-luciferin reaction to produce light emissions. The emitted light is proportional to the ATP concentration and this signal can be ready by plate readers such as the Envision Multilabel Reader (Perkin Elmer). ATP concentration monitoring can assess metabolic changes in cells and can also be used for quantitative evaluation of proliferation and cytotoxicity of cultured cells.

The cells of interest were prepared using methods described in Example 1 with the cell conditioning agent of choice. For example, rectal cancer cells were irradiated with 200 rads of radiation and allowed to recover for 48 hours. The radiation conditioned cells and control (non-irradiated cells) were both subjected to a test antibody raised against radiation conditioned colon carcinoma cells.

After 24 hours of antibody treatment of control and conditioned cells, the substrate for the ATPlite 1 step assay was prepared according to manufacturer's recommendations. 100 μl of substrate was added to each well. After the addition of substrate, the plates are shaken on an orbital shaker at 700 rpm for 2 minutes to ensure even lysis of the cells. Optionally, the plates can be sealed with a standard ELISA plate sealer before placement on the orbital shaker to prevent loss of fluid during shaking. Luminescence was measured using an Evision Multilabel plate reader.

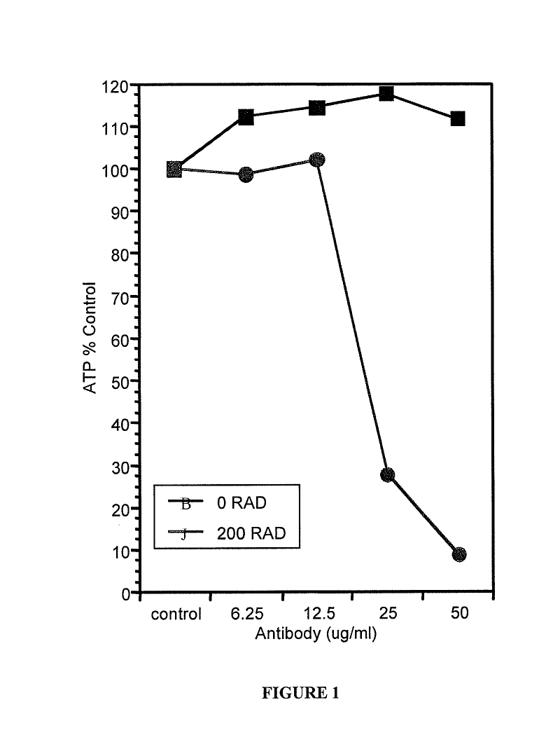

Radiation conditioned cells treated with test antibody had a drop in ATP concentration as compared to non-irradiated control cells treated with the same test antibody. These results show that the conditioned cells are metabolically susceptible to antigen modulators (test antibody) raised against radiation conditioned antigen targets. These results are also consistent with the idea that surface antigens may be present on conditioned cells and not on their non-conditioned counterparts, or that they may be inactive prior to radiation or other conditioning and upon activation by conditioning confer susceptibility of the tumor or diseased cell to the monoclonal antibody or other antigen modulator. Additionally, these surface antigens could be a target for antigen modifiers to modulate growth of the target conditioned cells. A graphical representation of these results are shown in FIG. 1.

The discovery of the novel activity of antigens and antigen modulators such as antibodies when developed against conditioned cells is a significant advance provided by this invention. The MAb presented in FIG. 1 would likely not have been detected as a potentially therapeutic antibody by conventional means of immunizing against prospective antigens that are believed to be disease specific or disease process specific, because of the negligible effect of that MAb in the absence of cellular pre-conditioning.

### Example 13

### In Vivo Treatment with Antibodies

The ability of the antibodies to reduce tumor growth in vivo can be assessed using tumor cells that are grafted under the kidney capsule in nude (nu/nu) mice. Cells of interest were prepared using methods described in Example 1 with the conditioning therapeutic of choice. Alternatively, cells of interest can be grafted and allowed to grow and conditioning therapeutic can then be introduced into the host animal. The cells of choice (either conditioned or unconditioned) are grafted under the kidney capsule of a nu/nu mouse. Such methods are well known in the art and by way of example disclosed in U.S. patent application Ser. No. 10/448,766. Generally, 5×10⁵ cells of choice in collagen gel is grafted under the kidney capsule. The graft was allowed to grow for 2 days. Test antibodies and vehicle control was injected intaperitoneally at a dose of 100 mg/kg on day 3. The conditioning therapeutic is introduced into the mouse at the desired concentration through intravenous injection. Antibodies and vehicle control is repeated every two days for three more doses. Three days after the final injection, the animals were euthanized and the kidneys with grafts were examined. The grafts and an area around them were then fixed and embedded in parafin blocks and sectioned through the entire graft area. Alternatively, quantitative PCR using human specific primers can also be used to analyze tumor growth.

A subcutaneous tumor model can also be used to assess the ability of the antibodies to reduce tumor growth in vivo. This model may be more appropriate in cases where the conditioning therapeutic is radiation. Cells of interest were prepared using methods described in Example 1 with the conditioning therapeutic of choice. Alternatively, unconditioned cells may also be used. Cells at the desired concentration (usually about 100 million cells per milliliter) were mixed with an equal volume of Matrigel® for a final injection volume of 0.1 ml. NCR.nu/nu homozygous mice were used and injected subcutaneously with the cell mixture. The mice were dosed intraperitoneally during the study with the test antibody and vehicle control. Conditioning therapeutic can be introduced into the mice at the appropriate dosage through intravenous injection. Alternatively, if the conditioning therapeutic is radiation, the site of the subcutaneous tumor can be irradiated at the desired dosage. Tumor growth can be evaluated over time to determine anti-tumor activity. Animals responding to antibody treatment can be maintained after treatment cessation to determine time to tumor regrowth.

### Example 14

### Cell Killing by Antibody on CA130 Cells with Radiation

Antibody 2 generated using the methods of the examples above and using irradiated colon cancer cells as an immunogen was tested in cell growth assays for synergistic effects of antibody plus radiation treatment. Lung adenocarcinoma cells (CA130) were seeded at 1×10⁶ cells/T75 flask and allowed to growth for 4 days. Cells were then irradiated at 200 Rad units or control (no irradiation). After irradiation, the cells were allowed to recover for 3 days. The cells were then lifted with collagen-dispase and plated on fibronectin-coated 96-well plates at 10,000 cells/well. 0.5 μg/ml of Antibody 2 was added concurrently with the cell plating. Cells were counted on Days 2, 3 and 4 after the addition of Antibody 2.

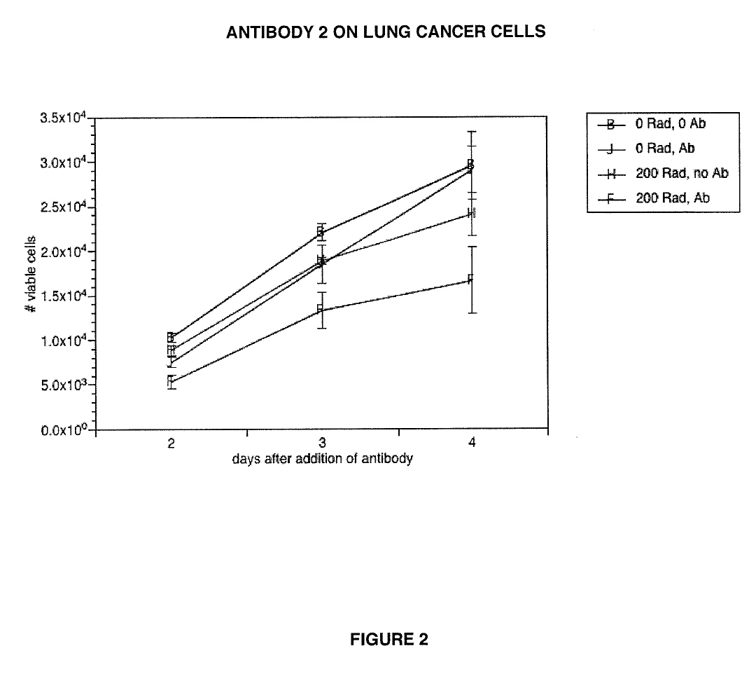

FIG. 2 shows a representative graph of the cell counts for (B) non-irradiated control cells with no antibody, (J) non-irradiated control cells with 0.5 μg/ml of Antibody 2 added, (H) irradiated cells with no antibody, and (F) irradiated cells with 0.5 μg/ml of Antibody 2 added. As can be seen in FIG. 2 from the Day 2 and 3 data points, antibody alone and radiation alone each decreased cell numbers. The treatment condition combining radiation and antibody treatment had a greater effect on cell survival than either treatment alone, and the effect was more than just additive. The data suggests that treatment with radiation and Antibody 2 were synergistic in their cell killing effect. This trend continued on day 4, where the cell number from the radiation plus antibody treatment condition was markedly lower than the other three conditions. Similar experiments were performed with Antibody 2 using other cell lines and other radiation conditions. The same trend of synergized cell killing was seen in these experiments.

### Example 15

### Cell Killing by Antibody on BRCA1103 Cells with Taxotere Treatment

Antibody 3 generated using the methods of the examples above and using taxotere treated breast cancer cells as an immunogen was tested in cell growth assays for synergistic effects of antibody and taxotere treatment. Breast carcinoma cells were seeded at 3000 cells/well on fibronectin coated 96-well plates. Concurrent with cell seeding, various concentrations of taxotere (0 to 0.625 ng/ml) and various concentrations of Antibody 3 (0 to 50 μg/ml) were added to each well in triplicates. Four days after treatment, the cells were assayed for cell growth using Alamar Blue reagent (Trek Diagnostics) according to manufacturer's instructions. Briefly, Alamar Blue reagent was added to each well at a 1/10th volume and incubated at 37° C. for 1-3 hours. The plate was read on a fluorescent plate reader and results are expressed in Relative Fluorescence Units (RFU).

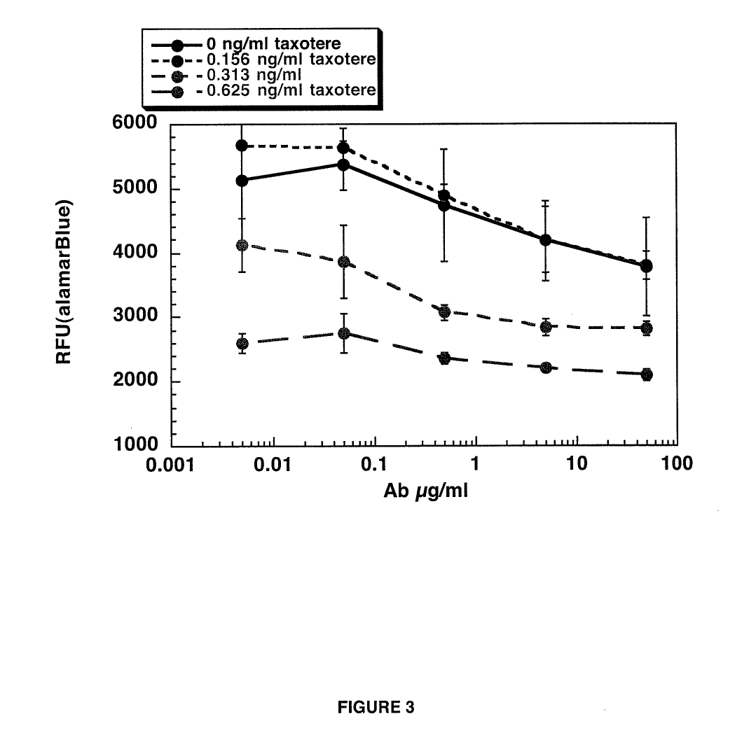

FIG. 3 shows a representative graph of the RFU for breast carcinoma cells treated with Antibody 3 and with taxotere. A decrease in RFU (corresponding to a decrease in cell number) was observed in the group with no taxotere plus increasing concentration of Antibody 3 conditions. This data suggests that Antibody 3 alone is able to reduce cell number in a concentration dependent manner. Further reduction in RFU was observed in the treatment group having antibody plus 0.313 ng/ml taxotere condition, and the data suggests that there may be an increased effect with both antibody and taxotere treatment. The 0.625 ng/ml taxotere plus antibody treatment group showed an even more pronounced reduction in RFU.

The data reflected in FIG. 3 suggest that there is a synergistic effect of Antibody 3 with taxotere, and that this effect is taxotere and antibody dose dependent. Similar experiments were performed with Antibody 3 and taxotere on other cells lines and the same trend of synergized cell killing was observed in these experiments.

### Example 16

### Identifying Molecular Mechanisms Using Proteomic Analysis

Protein Identification by Mass Spectrometry

The spots of interest are excised from the gel with a razor blade, placed in Eppendorf tubes, and de-stained by washing three times for 20 min in 50% v/v acetonitrile, 2.5 mM Tris, pH 8.5. The gel pieces are dehydrated at room temperature and covered with 10 iL of trypsin (0.04 mg/mL) in Tris buffer (2.5 mM, pH 8.5) and left at 37° C. overnight. The spots were crushed and peptides are extracted in 15 μL of 50% acetonitrile, 1% v/v formic acid. The extraction is conducted in an ultrasonic bath for 15 min. The sample is dehydrated at 30° C. for 3 h, 10 μL of 0.1% v/v formic acid are added and then Zip-Tip concentration/purification is performed. The extracted peptides are loaded onto the target plate by mixing 1 μL of each solution with the same volume of a matrix solution, prepared fresh every day by dissolving 10 mg/mL cyano-4-hydroxycinnamic acid in acetonitrile/ethanol (1:1 v:v), and allowed to dry. Measurements are performed using a TofSpec 2E MALDITOF instrument (Micromass, Manchester, UK), operated in reflectron mode, with an accelerating voltage of 20 kV. Peptide masses are searched against SWISS-PROT, TrEMBL, and NCBInr databases by utilizing the ProteinLynx program (Micromass) or NCBInr database by using the ProFound program.

For the proteins not identified by MALDI-TOF MS MS/MS analysis can be performed. In this case, the remainders of the tryptic digest samples are analyzed by reverse-phase nanoLC-MS/MS using a 1100 nanoflow LC system (Agilent Technologies Inc.). The LC-system is coupled to a QSTAR XL hybrid QqTOF mass spectrometer (Applied Biosystem,/MDS MDS-Sciex). Binding and chromatographic separation of peptides is achieved in a 15 cm long fused silica spray emitter (FS360-100-8-N-5-C15, New Objectives, Woburn, Mass., USA, 100 μm ID from Picotip New Objective) packed in-house with ReproSil-Pur C18-AQ 3 μm reverse phase resin. The tryptic peptide mixtures are loaded using an auto-sampler at a flow rate of 700 nL/min onto the packed column. The peptides are separated in a linear gradient of 5 to 24% acetonitrile in 0.5% acetic acid over 15 min and then in further 5 min to 81% acetonitrile at 300 nL/min. The QSTAR is operated in data dependent acquisition mode to automatically switch between MS and MS/MS. Protein identification is done using Mascot (Matrix Science, London, UK) searching the mammalian NCBI nonredundant protein database. Search parameters are: MS and MS/MS tolerance 0.2 Da, tryptic specificity allowing for up to 1 missed cleavages, fixed modification: carbamidomethylation of cysteine, variable modification: oxidation of methionine.

### Biochemical Function and Pathway Analysis

Proteins functions can be obtained from the SWISS-PROT Protein knowledge base (when available). Additionally, the software package PathwayAssist (Stratagene, La Jolla, Calif.) can be used to identify functional relationships among the proteins. This software explores gene interaction networks represented in the ResNet database. ResNet is a comprehensive database of molecular networks compiled natural language processing techniques applied to the whole PubMed database. The database contains more than 100 000 events of regulation, interaction and modification between 15 000 proteins, cell processes and small molecules.

What is claimed is:

1. A method of treating a disease, disorder or injury comprising administering to a subject in need thereof an effective amount of a cell conditioning agent and an effective amount of at least one modulator of an antigen displayed on the surface of a cell in said subject, wherein said antigen is altered in a diseased, damaged or injured cell relative to corresponding normal cells by said cell conditioning agent, thereby treating the disease, disorder or injury.

2. The method of claim 1, wherein the cell conditioning agent is selected from the group consisting of radiotherapeutic agents, radioisotopes, chemotherapeutic agents, infectious agents, heat shock agents, oxidative injury agents, growth factors that confer treatment resistance on a cell, and hormones that confer treatment resistance on a cell.

3. The method of claim 1, wherein the disease or injury is selected from the group consisting of cancer, infectious disease, inflammation, autoimnuune disease, cardiovascular disease, and neuronal disease.

4. The method of claim 1 wherein the modulator is selected from the group consisting of antibodies, antibody fragments, immunoconjugates, peptides, non-peptide small organic molecules, antisense molecules, inhibitory RNA molecules and oligonucleotide decoys.

5. The method of claim 4 wherein the antibody fragment is selected from the group consisting of Fab, Fab′, F(ab′)2, Fv fragments, diabodies, linear antibodies, single-chain antibody molecules; and multispecific antibodies formed from antibody fragments.

6. The method of claim 1 wherein said subject is human.

7. The method of claim 3 wherein said cancer is selected from the group consisting of adrenal gland tumors, AIDSassociated cancers, alveolar soft part sarcoma, astrocytic tumors, bladder cancer (squamous cell carcinoma and transitional cell carcinoma), bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, dhordoma, chromophobe renal cell carcinoma, clear cell carcinoma, colon cancer, colorectal cancer, cutaneous benign fibrous histiocytomas, desmoplastic small round cell tumors, ependymomas, Ewing's tumors, extraskeletal myxoid chondrosarcoma, fibrogenesis imperfecta ossium, fibrous dysplasia of the bone, gallbladder and bile duct cancers, gestational trophoblastic disease, germ cell tumors, head and neck cancers, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), medulloblastoma, melanoma, meningiomas, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, synovial sarcoma, testicular cancer, thymic carcinoma, thymoma, thyroid metastatic cancer, and uterine cancers (carcinoma of the cervix, endometrial carcinoma, and leiomyoma). In certain preferred embodiments, the cancerous cells are selected from the group of solid tumors including but not limited to breast cancer, colon cancer, prostate cancer, lung cancer, sarcoma, renal metastatic cancer, thyroid metastatic cancer, and clear cell carcinoma.

8. The method of claim 2, wherein the infectious agent is a pathogen selected from the group consisting of virus, bacterium, prions, protozoa, viroid, intracellular parasites and fungus.

9. The method of claim 4 wherein the antibody is humanized.

10. The method of claim 4 wherein the antibody is human.

11. The method of claim 1 wherein said administration is concurrent.

12. The method of claim 1 wherein said administration is consecutive.

13. The method of claim 1 wherein said subject is treated with said cell conditioning agent first, followed by treatment with said modulator.

14. The method of claim 1 wherein treatment with said cell conditioning agent continues concurrently with the treatment with said modulator.

15. The method of claim 1, wherein said cell conditioning agent is administered at a subtherapeutic level.

16. The method of claim 1, wherein said modulator carries a toxin moiety that is sensitive to local release from said modulator upon administration of said cell conditioning agent.

17. The method of claim 1, wherein said cell conditioning agent is radiation and said modulator is an antibody.

18. The method of claim 1, wherein said antigen is proteinaceous, partially-proteinaceous or non-proteinaceous.

19. The method of claim 18, wherein said antigen is selected from the group consisting of carbohydrates, lipids, hydrophilic phosphorous molecules and nucleic acids.

20. The method of claim 1, wherein said alteration is quantitative or qualitative.

21. The method of claim 22, wherein the alteration in said cell is by a process or event selected from the group consisting of transcriptional regulation, differential RNA splicing, post-transcriptional modifications, post-translational modifications, mutations introduced during the transcription or the translation process, extracellular protease exposure, novel hetero-dimer formation, altered glycosylation, altered phosphorylation, altered acetylation, altered methylation, altered biotinylation, altered glutamylation, altered glycylation, altered isoprenylation, altered lipoylation, altered phosphopantetheinylation, altered sulfation, altered ISGylation, altered SUMOylation, altered ubiquitination, altered citrullination, altered deamidation, altered disulfide bridges, altered proteolytic cleavage, altered translocation, changes in protein turnover, protein aggregation, oxidation, lipid aggregation, altered conformation, and biochemical changes in the cell membrane.

22. The method of claim 1, wherein the modulator binds to, agonizes or antagonizes at least one activity of the antigen.

23. The method of claim 1, comprising administering two or more modulators.

24. The method of claim 23, wherein each of the modulators bind to, agonize or antagonize at least one activity of a different antigen.

25. The method of claim 1, wherein the modulator binds to, agonizes or antagonizes at least one activity of an antigen displayed on fetal cells exposed to the conditioning agent.

26. The method of claim 25, wherein the fetal cells are stem cells or progenitor cells.

27. A method for inhibiting the proliferation of tumor cells comprising: (a) determining the presence of at least one antigen displayed on the surface of a cell which is altered in said tumor cells relative to normal cells by a cell conditioning agent; and (b) treating said tumor cells with said cell conditioning agent and a modulator of at least one of said antigens.

28. A method of claim 27, wherein the alteration in said tumor cells is by a process or event selected from the group consisting of transcriptional regulation, differential RNA splicing, post-transcriptional modifications, post-translational modifications, mutations introduced during the transcription or the translation process, extracellular protease exposure, novel hetero-dimer formation, altered glycosylation, altered phosphorylation, altered acetylation, altered methylation, altered biotinylation, altered glutamylation, altered glycylation, altered isoprenylation, altered lipoylation, altered phosphopantetheinylation, altered sulfation, altered ISGylation, altered SUMOylation, altered ubiquitination, altered citrullination, altered deamidation, altered disulfide bridges, altered proteolytic cleavage, altered translocation, changes in protein turnover, protein aggregation, oxidation, lipid aggregation, altered conformation, and biochemical changes in the cell membrane.

29. A method for diagnosing the degree to which a subject has been exposed to a cell conditioning agent, comprising (a) obtaining a sample of cells from said subject;
and (b) exposing said cells to at least one modulator of an antigen displayed on the surface of a cell the presence of which is altered in a target cell relative to normal cells by said cell conditioning agent.

30. The method of claim 29, wherein said cell sample is exposed to a panel of at least one modulator, each such modulator being specific for at least one antigen displayed on the surface of a cell the presence of which is altered in a target cell relative to normal cells by varying exposures to said cell conditioning agent.

31. The method of claim 29, wherein the cell sample is selected from the group consisting of bone marrow cells, peripheral white blood cells, red blood cells, tumor cells, and endothelial cells.

32. A method for assessing the effects on a subject of the administration of a cell conditioning agent, comprising (a) obtaining a sample of cells from said subject; and (b) determining the presence in the sample of at least one antigen displayed in the surface of cell the presence of which is altered in a target cell relative to normal cells by said cell conditioning agent.

33. The method of claim 32, wherein said cell sample is selected from the group consisting of bone marrow cells, peripheral white blood cells, red blood cells, tumor cells, and endothelial cells.

34. The method of claim 32, wherein the presence of said antigen is determined by using a modulator specific for said antigen.

35. The method of claim 32, wherein the presence of said at least one antigen is used as a biomarker to assess the degree to which an efficacious dose of said cell conditioning agent has been attained.

36. A method for selecting responsive patients for treatment with a cell conditioning agent, comprising (a) administering to a subject a therapeutic cell conditioning agent, (b) administering to said subject at least one modulator of an antigen displayed in the surface of a cell the presence of which is altered in a target cell relative to normal cells by a cell conditioning agent, which modulator is detectably labeled; and (c) assessing said subject for the presence of said detectable label.

37. The method of claim 36, wherein said modulator is detectably labeled with marker selected from the group consisting of radioisotopes, fluorescent agents, enzyme-linked agents, color-changing agents, and bioluminescent agents, and affinity-based agents.

38. The method of claim 36, wherein said assessment indicates the presence of said detectable label in an identifiable location in said subject, and wherein said method is followed by the additional step of treating said subject with an effective amount of a cell conditioning agent, and a modulator of said antigen the presence of which is altered in a diseased, damaged or injured cell relative to corresponding normal cells by said cell conditioning agent.

39. The method of claim 38, wherein said modulators are the same.

40. The method of claim 38, wherein said modulators are different.

41. The method of claim 38 wherein said administration is concurrent.

42. The method of claim 38 wherein said administration is consecutive.

43. A method for the protection of normal cells in a subject that are located near diseased or damaged cells, comprising administering to said subject (a) an effective amount of a cell conditioning agent, and (b) a modulator of an antigen displayed on the surface of a cell the presence of which is altered in a normal cell relative to a corresponding diseased, damaged or injured cell by said cell conditioning agent.

44. The method of claim 43, wherein said modulator is an agonist agent providing protective stimulation to said normal cells.

45. The method of claim 43, wherein said diseased or damaged cells are neoplastic.

46. A method for inhibiting the proliferation of diseased or damaged cells in a subject, comprising administering to normal cells in a subject that are located near to said diseased or damaged cells (a) an effective amount of a cell conditioning agent, and (b) a modulator of an antigen displayed on the surface of a cell the presence of which is altered in a normal cell relative to a corresponding diseased, damaged or injured cell by said cell conditioning agent.

47. The method of claim 46, wherein said diseased or damaged cells are neoplastic.

48. The method of claim 46, wherein said normal cells are endothelial.

49. The method of claim 46, wherein the administration continues for a sufficient period to inhibit the function of the vasculature present in said diseased or damaged cells.

49. The method of claim 46, wherein the administration continues for a sufficient period to inhibit the function of the vasculature present in said diseased or damaged cells.

50. A method for identifying an antigen target for disease treatment comprising: (a) contacting a cell with an effective amount of a cell conditioning agent; (b) determining a profile of antigens displayed on the surface of said cell; and (c) identifying as a target for disease treatment an antigen the presence of which is altered by said cell conditioning agent relative to its presence in a corresponding untreated cell.

51. The method of claim 50, wherein the profile is created by immunizing an immune cell with the contacted cell or membrane fragments therefrom.

52. A method for identifying a gene target for disease treatment comprising: (a) contacting a cell with an effective amount of a cell conditioning agent that is not an effective level of a chemotherapeutic agent; (b) determining the gene expression profile of said cell; and (c) identifying as a target for disease treatment a gene the expression of which is altered by said cell conditioning agent relative to its expression in a corresponding untreated cell.

53. A method for the treatment of a disease in a mammalian subject comprising the steps of: (a) incubating a diseased cell with an effective dose of a cell conditioning agent; (b) determining the gene expression profile of said disease cell prior to and following said incubation; (c) identifying a gene the expression of which is enhanced by said cell conditioning agent; and (d) treating said patient with said cell conditioning agent and a modulator targeting said gene.

54. The method of any of claims 50-53, wherein said cell conditioning agent is selected from the group consisting of infectious agents, hypoxia, heat shock agents, oxidation injury, radiotherapeutic agents, radioisotopes, chemotherapeutic agents, on a neoplastic cell the presence of which is altered in said neoplastic cell relative to corresponding normal cells by said cell conditioning agent.

56. A method for immunizing a host mammal or an antibody-producing cell to produce an antibody that binds to a cell surface antigen the presence of which is selectively altered in a diseased, damaged or injured cell relative to corresponding normal cells by a cell conditioning agent, comprising: contacting the mammal or the antibody-producing cell with a cell conditioned by said cell conditioning agent or a membrane fragment thereof under conditions suitable for eliciting an immune response.

57. A method of maintaining or increasing cell susceptibility to a therapeutic agent comprising (a) delivering to a subject in need thereof an effective amount of a modulator specific for a cell surface antigen the presence of which is altered in a cell relative to untreated cells by said therapeutic agent; and (b) treating said subject with said therapeutic agent.

58. A method of generating an antibody specific for a surface antigen presented by a conditioned cell, comprising
a) immunizing an antibody-producing cell with said conditioned cell or a membrane fragment thereof to elicit an immune response culminating in production of said antibody specific for said surface antigen; and
b) culturing said antibody-producing cell under conditions such that said antibody is produced.

59. A method of preparing antigen-presenting cells, comprising exposing diseased cells to a cell conditioning agent, said exposure being sufficient for the altered appearance of at least one cell surface antigen that is altered in expression level, or has been subjected to altered post-translational modification relative to the appearance of said cell surface antigen on a corresponding untreated cell.

60. A method for the preparation of a cellular vaccine, in which diseased cells suitable for use as a vaccine are exposed to a cell conditioning agent, said exposure being sufficient for the appearance of at least one cell surface antigen that has been determined to be altered in a cell relative to untreated cells by said cell conditioning agent.

61. A method for detecting genotypic changes in a cell, comprising profiling the cell surface antigen phenotype of a cell that has been exposed to a cell conditioning agent.

62. A therapeutic composition comprising an effective amount of a cell conditioning agent and a modulator specific for a cell surface antigen the presence of which has been determined to be altered in a cell relative to untreated cells by said cell conditioning agent.

63. A therapeutic composition comprising an effective amount of a sub-therapeutic dose of a radiotherapeutic agent and a modulator specific for a cell surface antigen the presence of which has been determined to be selectively altered in a cell relative to untreated cells by said radiotherapeutic agent.

64. A composition for vaccinating against tumors comprising (a) a cell conditioning agent and (b) at least one tumor cell that is expressing a cell surface antigen the presence of which has been determined to be altered in a cell relative to untreated cells by the exposure of said tumor cell to said cell conditioning agent.

65. A packaged pharmaceutical for treating a patient to reduce proliferation of and/or kill target cells that present an antigen, the presence of said antigen being altered in a cell relative to untreated cells by a cell conditioning agent, comprising (a) an antibody formulation immunoreactive with said antigen, which is accessible on target cells; (b) a cell conditioning agent, and (c) optional instructions for using the antibody formulation in conjunction with said cell conditioning agent to reduce proliferation of and/or kill target cells.

66. A method of identifying antigens presented or displayed on the surface of a diseased, damaged or injured cell, comprising:
(a) exposing said diseased, damaged or injured cell to at least one cell conditioning agent; and
(b) selecting antigen binding molecules that bind to at least one antigen presented or displayed on the conditioned diseased, damaged or injured cell, wherein said at least one antigen is altered in the diseased, damaged or injured cell relative to corresponding normal cells by exposure to said cell conditioning agent.

67. The method of claim 66, wherein the selecting comprises immunizing a subject or antibody producing cell with said conditioned diseased, damaged or injured cell or a cellular membrane fragment therefrom.

68. The method of claim 66, wherein the selecting comprises screening a library of antigen binding molecules for binding to said conditioned diseased, damaged or injured cell or a cellular membrane fragment therefrom.

69. A method of identifying a modulator of an antigen presented or displayed on the surface of a diseased, damaged or injured cell, comprising:
(a) exposing said diseased, damaged or injured cell to at least one cell conditioning agent; and
(b) selecting antigen binding molecules that bind to at least one antigen presented or displayed on the conditioned diseased, damaged or injured cell, wherein said at least one antigen is altered in the diseased, damaged or injured cell relative to corresponding normal cells by exposure to said cell conditioning agent.

70. The method of claim 69, wherein the selecting comprises immunizing a subject or antibody producing cell with said conditioned diseased, damaged or injured cell or a cellular membrane fragment therefrom.

71. The method of claim 69, wherein the selecting comprises screening a library of antigen binding molecules for binding to said conditioned diseased, damaged or injured cell or a cellular membrane fragment therefrom.

72. The method of claim 68 or 71, wherein the library is screened against an array of cells, or cellular membrane fragments therefrom, wherein the array comprises conditioned cells exposed to varying concentrations or amounts of at least one cellular conditioning agent, or cellular membrane fragments therefrom.

73. The method of claim 72, wherein the array comprises conditioned cells exposed to at least one cellular conditioning agent at varying times, or cellular membrane fragments therefrom.

74. The method of claim 66 or 69, wherein the alteration in said cell is by a process or event selected from the group consisting of transcriptional regulation, differential RNA splicing, post-transcriptional modifications, post-translational modifications, mutations introduced during the transcription or the translation process, extracellular protease exposure, novel hetero-dimer formation, altered glycosylation, altered phosphorylation, altered acetylation, altered methylation, altered biotinylation, altered glutamylation, altered glycylation, altered isoprenylation, altered lipoylation, altered phosphopantetheinylation, altered sulfation, altered ISGylation, altered SUMOylation, altered ubiquitination, altered citrullination, altered deamidation, altered disulfide bridges, altered proteolytic cleavage, altered translocation, changes in protein turnover, protein aggregation, oxidation, lipid aggregation, altered conformation, and biochemical changes in the cell membrane.

75. The composition of claim 64, comprising a plasma membrane preparation isolated from said tumor cell.
